(19) United States
(12) Patent Application Publication
(10) Pub. No.: US 2012/0201787 A1
(43) Pub. Date: Aug. 9, 2012

(54) TREATMENT OF SPINAL CORD INJURY AND TRAUMATIC BRAIN INJURY USING AMNION DERIVED ADHERENT CELLS

(76) Inventors: Stewart Abbot, Warren, NJ (US); James W. Edinger, Belford, NJ (US); Aleksandar Francki, Annandale, NJ (US); Vladimir Jankovic, New York, NY (US); Aleksandr Kaplunovsky, Budd Lake, NJ (US); Kristen Labazzo, Springfield, NJ (US); Eric Law, East Brunswick, NJ (US); Bitao Liang, Closter, NJ (US)

(21) Appl. No.: 13/327,289

(22) Filed: Dec. 15, 2011

Related U.S. Application Data
(60) Provisional application No. 61/424,596, filed on Dec. 17, 2010.

(51) Int. Cl. A61K 35/50; A61P 25/28; A61P 27/02; A61P 25/08; A61P 1/08; A61P 27/00; A61P 25/00; A61P 25/24

(52) U.S. Cl. 424/93.7; 424/528

(57) ABSTRACT

Provided herein are methods of treating spinal cord and traumatic brain injuries using cells from amnion, and populations of such cells, referred to herein as “amnion derived adherent cells” (“AMDACs”).

This application claims priority to U.S. provisional application No. 61/424,596, filed Dec. 17, 2010, the disclosure of which is herein incorporated by reference in its entirety.

### 1. FIELD

Provided herein are methods of treating spinal cord and traumatic brain injuries using cells from amnion, and populations of such cells, referred to herein as “amnion derived adherent cells” (“AMDACs”).

### 2. BACKGROUND

Central Nervous System (CNS) injuries represent a medically important problem. Approximately 300,000 people living in the United States suffer from spinal cord injury (SCI) and, each year, approximately 10,000-14,000 new cases of SCI are diagnosed. SCI usually results from trauma to the vertebral column, e.g., as a result of displaced bone or disc compressing the spinal cord. SCI can occur without obvious vertebral fractures, for example, from loss of blood flow to the spinal cord, and spinal fractures can occur without spinal cord injury.

Traumatic brain injury (TBI) is one of the leading causes of disability and death among young adults around the world. In military situations, for example, brain damage results from, e.g., direct impact, penetrating objects such as bullets and shrapnel, and from blast waves caused by explosions.

### 3. SUMMARY

Provided herein are methods for the treatment of an individual having an injury to the CNS, e.g., a spinal cord injury or traumatic brain injury, comprising administering to the individual having the CNS injury one or more doses of amnion derived adherent cells (“AMDACs”).

In one aspect, provided herein are methods of treating an individual having, or experiencing, a symptom of or a condition or syndrome related to, a spinal cord injury (SCI), comprising administering to the individual a therapeutically effective amount of AMDACs, or medium conditioned by AMDACs, wherein the therapeutically effective amount is an amount sufficient to cause a detectable improvement in one or more symptoms of, or a reduction in the progression of one or more symptoms of, said spinal cord injury.

In some embodiments, the therapeutically effective amount of AMDACs, or culture medium conditioned by AMDACs is administered to the individual within 1, 2, 3, 4, 5, 6, 7, 8, 9, 10, 11, 12, 13, 13, 15, 16, 17, 18, 19, 20, 25, 30, 35, 40, 45, 50 days or more of injury, or within 1, 2, 3, 4, 5, 6, 7, 8, 9, 10 or more years after the CNS injury.

In a specific embodiment, the CNS injury is a spinal cord injury (SCI). In some embodiments, the spinal cord injury is caused by direct trauma. In some embodiments, the spinal cord injury is caused by compression by bone fragments or disc material. In some embodiments, the spinal cord injury is at one or more of the cervical vertebrae, thoracic vertebrae, lumbar vertebrae, or sacral vertebrae. In some embodiments, the spinal cord injury is to one or more of the cervical cord, thoracic cord, lumbrosacral vertebrae, conus, occiput, or one or more nerves of the cauda equina.

In some embodiments, provided herein are methods of treating a disease, disorder or condition associated with CNS injury. In some embodiments, the disease, disorder or condition associated with CNS injury is spinal shock resulting from a spinal cord injury. In some embodiments, the disease, disorder or condition associated with CNS injury is neurogenic shock resulting from a spinal cord injury. In some embodiments, the disease, disorder or condition associated with CNS injury is autonomic dysreflexia resulting from a spinal cord injury. In some embodiments, the disease, disorder or condition associated with CNS injury is edema resulting from a spinal cord injury. In some embodiments, the disease, disorder or condition associated with CNS injury is selected from the group consisting of central cord syndrome, Brown-Séquard syndrome, anterior cord syndrome, conus medullaris syndrome, and cauda equina syndrome.

In some embodiments, the therapeutically effective amount of AMDACs or medium conditioned by AMDACs administered is an amount sufficient to cause a detectable improvement in, or a reduction in the progression of, one or more of the following symptoms of spinal cord injury: loss or impairment of motor function, sensory function, or motor and sensory function, in the cervical, thoracic, lumbar or sacral segments of the spinal cord. In some embodiments, the one or symptoms of the spinal cord injury comprises loss or impairment of motor function, sensory function, or motor and sensory function, in the arms, trunk, legs or pelvic organs. In some embodiments, the one or symptoms of the spinal cord injury comprises numbness in one or more of dermatomes C1, C2, C3, C4, C5, C6, C7, T1, T2, T3, T4, T5, T6, T7. T8, T9, T10, T11, T12, L1, L2, L3, L4 or L5.

In some embodiments of treating SCI provided herein, the method further comprises administering a second therapeutic agent to said individual. In some embodiments, the second therapeutic agent is a corticosteroid, a neuroprotective agent, an immunomodulatory or immunosuppressant agent, or an anticoagulant.

In another specific embodiment of the methods of treatment provided herein, the disease, disorder or condition associated with CNS injury is a traumatic brain injury. In some embodiments, the traumatic brain injury is an injury to the frontal lobe, parietal lobe, occipital lobe, temporal lobe, brain stem, or cerebellum. In some embodiments, the traumatic brain injury is a mild traumatic brain injury. In some embodiments, the traumatic brain injury is a moderate to severe traumatic brain injury.

In some embodiments, the therapeutically effective amount of AMDACs, or medium conditioned by AMDACs administered is an amount sufficient to cause a detectable improvement in, or a reduction in the progression of, one or more of the following symptoms of mild traumatic brain injury: headache, memory problems, attention deficits, mood swings and frustration, fatigue, visual disturbances, memory loss, poor attention/concentration, sleep disturbances, dizziness/loss of balance, irritability, emotional disturbances, feelings of depression, seizures, nausea, loss of smell, sensitivity to light and sounds, mood changes, getting lost or confused, or slowness in thinking.

In some embodiments, the therapeutically effective amount of AMDACs, or medium conditioned by AMDACs administered is an amount sufficient to cause a detectable improvement in, or a reduction in the progression of, one or more of the following symptoms of moderate to severe traumatic brain injury: difficulties with attention, difficulties with concentration, distractibility, difficulties with memory, slowness of speed of processing, confusion, perseveration, impulsiveness, difficulties with language processing, difficulties with speech and language, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, difficulty with the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), a decrease or loss of hearing, ringing in the ears (tinnitus), increased sensitivity to sounds, loss or diminished sense of smell (anosmia), loss or diminished sense of taste, seizures, convulsions associated with epilepsy, physical paralysis/spasticity, chronic pain, loss of control of bowel and/or bladder, sleep disorders, loss of stamina, appetite changes, dysregulation of body temperature, menstrual difficulties, social-emotional difficulties, dependent behaviors, lack of emotional ability, lack of motivation, irritability, aggression, depression, disinhibition, or lack of awareness.

In some embodiments of treating TBI provided herein, the method further comprises administering a second therapeutic agent to said individual. In some embodiments, the second therapeutic agent is an anti-seizure drug, an antidepressant, amantadine, methylphenidate, bromocriptine, carbamamazapine or amitriptyline.

In some embodiments of treating a CNS injury, e.g., a spinal cord injury or traumatic brain injury, as provided herein, the therapeutically effective amount of AMDACs, or culture medium conditioned by AMDACs is administered to the individual by a route selected from the group consisting of intravenous, intraarterial, intraperitoneal, intraventricular, intrasternal, intracranial, intramuscular, intrasynovial, intraocular, intravitreal, intracerebral, intracerebroventricular, intrathecal, intraosseous infusion, intravesical, transdermal, intracisternal, epidural, lumbar puncture, cisterna magna or subcutaneous administration. In some embodiments, wherein the therapeutically effective amount of AMDACs, or culture medium conditioned by AMDACs is administered to the individual directly into the site of the injury.

In a specific embodiment, the TBI treated in accordance with the methods described herein results from or is caused by a non-ischemic event. In another specific embodiment, the TBI treated in accordance with the methods described herein is not a hematoma or does not result from a hematoma. In another specific embodiment, the TBI treated in accordance with the methods described herein is not a hematoma that caused by external force on the skull. In another specific embodiment, the TBI treated in accordance with the methods described herein is not caused by a disruption of the flow of blood in or around the brain of the individual suffering from the TBI.

In certain embodiments, provided herein is a method of inhibiting a pro-inflammatory response to a CNS injury in an individual, for example a spinal cord injury or traumatic brain injury, comprising contacting T cells (e.g., CD4⁺ T lymphocytes or leukocytes) that are associated with or part of the CNS injury with AMDACs, e.g., the AMDACs described herein. In a specific embodiment, the inflammatory response is a Th1 response or a Th17 response. In a specific embodiment, said contacting detectably reduces Th1 cell maturation. In a specific embodiment of the method, said contacting detectably reduces the production of one or more of interleukin-1β (IL-1β), IL-12, IL-17, IL-21, IL-23, tumor necrosis factor alpha (TNFα) and/or interferon gamma (IFNγ) by said T cells. In another specific embodiment of the method, said contacting potentiates or upregulates a regulatory T cell (Treg) phenotype. In another specific embodiment, said contacting downregulates dendritic cell (DC) and/or macrophage expression of markers (e.g., CD80, CD83, CD86, ICAM-1, HLA-II) that promote Th1 and/or Th17 immune response. In a specific embodiment, said T cells are also contacted with IL-10, e.g., exogenous IL-10 or IL-10 not produced by said T cells, e.g., recombinant IL-10. In another embodiment, provided herein is a method of reducing the production of pro-inflammatory cytokines from macrophages, comprising contacting the macrophages with an effective amount of AMDACs. In another embodiment, provided herein is a method of upregulating tolerogenic cells and/or cytokines, e.g., from macrophages, comprising contacting immune system cells with an effective amount of AMDACs. In a specific embodiment, said contacting causes activated macrophages to produce detectably more IL-10 than activated macrophages not contacted with said AMDACs. In another embodiment, provided herein is a method of upregulating, or increasing the number of, anti-inflammatory T cells, comprising contacting immune system cells with an effective amount of AMDACs.

In one embodiment, provided herein is a method of inhibiting a CNS injury-associated Th1 response in an individual comprising administering to the individual an effective amount of AMDACs, wherein said effective amount is an amount that results in a detectable decrease in said CNS injury-associated Th1 response in the individual. In another embodiment, provided herein is a method of inhibiting a CNS injury-associated Th17 response in an individual comprising administering to the individual an effective amount of AMDACs, wherein said effective amount is an amount that results in a detectable decrease in a Th17 response in the individual. In specific embodiments of these methods, said administering detectably reduces the production, by T cells, or an antigen presenting cell (e.g., DC, macrophage or monocyte) in said individual, of one or more of lymphotoxins-1α (LT-1α), IL-1β, IL-12, IL-17, IL-21, IL-23, TNFα and/or IFNγ. In another specific embodiment of the method, said contacting potentiates or upregulates a regulatory T cell (Treg). In another embodiment, said contacting modulates (e.g., reduces) production by dendritic cells (DC) and/or macrophages in said individual of markers that promote a Th1 or Th17 response (e.g. CD80, CD83, CD86, ICAM-1, HLA-II). In another specific embodiment, the method comprises additionally administering IL-10 to said individual.

In another aspect, provided herein are AMDACs, as described herein, that have been genetically engineered to express one or more anti-inflammatory cytokines. In a specific embodiment, said anti-inflammatory cytokines comprise IL-10.

The AMDACs described herein may be identified by different combinations of cellular and genetic markers. In a specific embodiment, for example, AMDACs are OCT-4⁻ as determinable by reverse-transcriptase-polymerase chain reaction (RT-PCR). In another embodiment, the AMDACs are CD49f⁺, as determinable by flow cytometry. In yet another embodiment, the AMDACs are OCT-4⁻ and CD49f⁺ as determinable by RT-PCR and flow cytometry, respectively. In still another embodiment, the AMDACs are CD49f⁺, CD105⁺, and CD200⁺ as determinable by immunolocalization, e.g., flow cytometry. In another embodiment, the AMDACs are OCT-4⁻ as determinable by RT-PCR and CD49f⁺, CD105⁺, and CD200⁺ as determinable by immunolocalization, e.g., flow cytometry. In another specific embodiment, said AMDACs are positive for VEGFR1/Flt-1 (vascular endothelial growth factor receptor 1) and/or CD309 (also known as vascular endothelial growth factor receptor 2 (VEGFR2)/KDR), as determinable by immunolocalization, e.g., flow cytometry. In another specific embodiment, said AMDACs are CD90⁺ and/or CD117⁻ as determinable by flow cytometry, and/or HLA-G−, as determinable by RT-PCR. In another specific embodiment, said AMDACs are OCT-4⁻ and HLA-G⁻, as determinable by RT-PCR, and CD49f⁺, CD90⁺, CD105⁺, and CD117⁻ as determinable by flow cytometry. In another specific embodiment, any of the above AMDACs are additionally one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, or CXCR4⁻ (chemokine (C—X—C motif) receptor 4) as determinable by immunolocalization, e.g., flow cytometry. In another specific embodiment, any of the above AMDACs are additionally CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry.

In another specific embodiment, the AMDACs are GFAP⁺ as determinable by a short-term neural differentiation assay (see, e.g., Section 5.12.1, below). In another specific embodiment, the AMDACs are beta-tubulin III (Tuj1)⁺ as determinable by a short-term neural differentiation assay (see, e.g., Section 5.12.1, below). In another specific embodiment, the AMDACs are OCT-4⁻, GFAP⁺, and beta-tubulin III (Tuj1)⁺. In another specific embodiment, the AMDACs described herein are CD200⁺, CD105⁺, CD90⁺, and CD73⁺. In another specific embodiment, AMDACs described herein are CD117⁻ and are not selected using an antibody to CD117. In another specific embodiment, the AMDACs described herein are CD146⁻ and are not selected using an antibody to CD146. In another specific embodiment, the AMDACs described herein are OCT-4⁻ and do not express CD34 following induction with VEGF as determinable by RT-PCR and/or immunolocalization (e.g., flow cytometry). In another specific embodiment, the AMDACs described herein are neurogenic, as determinable by a short-term neural differentiation assay (see, e.g., Section 5.12.1, below). In another specific embodiment, the AMDACs described herein are non-chondrogenic as determinable by an in vitro chondrogenic potential assay (see, e.g., Section 5.12.3, below). In another specific embodiment, the AMDACs described herein are non-osteogenic as determinable by an osteogenic phenotype assay (see, e.g., Section 5.12.2, below). In another specific embodiment, the AMDACs described herein are non-osteogenic after being cultured for up to 6 weeks (e.g., for 2 weeks, for 4 weeks, or for 6 weeks) in DMEM at pH 7.4 (High glucose) supplemented with 100 nM Dexamethasone, 10 mM β-glycerol phosphate, 50 μM L-ascorbic acid-2-phosphate, wherein osteogenesis is assessed using von Kossa staining; alizarin red staining; or by detecting the presence of osteopontin, osteocalcin, osteonectin, and/or bone sialoprotein by, e.g., RT-PCR.

In another specific embodiment, any of the above AMDACs additionally: (a) express one or more of CD9, CD10, CD44, CD54, CD98, CD200, Tie-2, TEM-7, VEGFR1/Flt-1, or VEGFR2/KDR (CD309), as determinable by immunolocalization, e.g., flow cytometry; (b) lack expression of one or more of CD31, CD34, CD38, CD45, CD133, CD143, CD144, CD146, CD271, CXCR4, HLA-G, or VE-cadherin, as determinable by immunolocalization, e.g., flow cytometry; (c) lack expression of SOX2, as determinable by RT-PCR; (d) express mRNA for one or more of ACTA2, ADAMTS1, AMOT, ANG, ANGPT1, ANGPT2, ANGPTL1, ANGPTL2, ANGPTL4, BAI1, c-myc, CD44, CD140a, CD140b, CD200, CD202b, CD304, CD309, CEACAM1, CHGA, COL15A1, COL18A1, COL4A1, COL4A2, COL4A3, Connexin-3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, Galectin-1, GRN, HGF, HEY1, HSPG2, IFNB1, IL8, IL12A, ITGA4, ITGAV, ITGB3, KLF-4, MDK, MMP2, MYOZ2, NRP2, PDGFB, PF4, PGK1, PROX1, PTN, SEMA3F, SERPINB5, SERPINC1, SERPINF1, TGFA, TGFB1, THBS1, THBS2, TIE1, TIMP2, TIMP3, TNF, TNNC1, TNNT2, TNFSF15, VASH1, VEGF, VEGFB, VEGFC, or VEGFR1/FLT1; (e) produce one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; (0 secrete one or more of vascular endothelial growth factor (VEGF), hepatocyte growth factor (HGF), interleukin-8 (IL-8), monocyte chemotactic protein-3 (MCP-3), FGF2, Follistatin, G-CSF, EGF, ENA-78, GRO, IL-6, MCP-1, PDGF-BB, TIMP-2, uPAR, or galectin-1 into culture medium in which the AMDACs grows; (g) express one or more of micro RNAs miR-17-3p, miR-18a, miR-18b, miR-19b, miR-92, or miR-296 at a higher level than an equivalent number of bone marrow-derived mesenchymal stem cells; (h) express one or more of micro RNAs miR-20a, miR-20b, miR-221, miR-222, miR-15b, or miR-16 at a lower level than an equivalent number of bone marrow-derived mesenchymal stem cells; (i) express one or more of miRNAs miR-17-3p, miR-18a, miR-18b, miR-19b, miR-92, miR-20a, miR-20b, miR-296, miR-221, miR-222, miR-15b, and/or miR-16; or (j) express increased levels of one or more of CD202b, IL-8 or VEGF when cultured in less than about 5% O₂ compared to expression of CD202b, IL-8 or VEGF when cultured under 21% O₂. In a more specific embodiment, said AMDACs are OCT-4⁻, as determinable by RT-PCR, and CD49f⁺, HLA-G⁻, CD90⁺, CD105⁺, CD117⁻, and CD200⁺, as determinable by immunolocalization, e.g., flow cytometry. In another specific embodiment, said AMDACs are OCT-4⁻, as determinable by RT-PCR, and CD49f⁺, HLA-G⁻, CD90⁺, CD105⁺, and CD117⁻, as determinable by immunolocalization, e.g., flow cytometry, and wherein said AMDACs additionally: (a) express CD9, CD10, CD44, CD54, CD98, CD200, Tie-2, TEM-7, VEGFR1/Flt-1, and VEGFR2/KDR (CD309), as determinable by immunolocalization, e.g., flow cytometry; (b) lack expression of CD31, CD34, CD38, CD45, CD133, CD143, CD144, CD146, CD271, CXCR4, HLA-G, and VE-cadherin, as determinable by immunolocalization, e.g., flow cytometry; (c) lack expression of SOX2, as determinable by RT-PCR; (d) express mRNA for ACTA2, ADAMTS1, AMOT, ANG, ANGPT1, ANGPT2, ANGPTL1, ANGPTL2, ANGPTL4, BAI1, c-myc, CD44, CD140a, CD140b, CD200, CD202b, CD304, CD309, CEACAM1, CHGA, COL15A1, COL18A1, COL4A1, COL4A2, COL4A3, Connexin-3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, Galectin-1, GRN, HGF, HEY1, HSPG2, IFNB1, IL8, IL12A, ITGA4, ITGAV, ITGB3, KLF-4, MDK, MMP2, MYOZ2, NRP2, PDGFB, PF4, PGK1, PROX1, PTN, SEMA3F, SERPINB5, SERPINC1, SERPINF1, TGFA, TGFB1, THBS1, THBS2, TIE1, TIMP2, TIMP3, TNF, TNNC1, TNNT2, TNFSF15, VASH1, VEGF, VEGFB, VEGFC, and VEGFR1/FLT1 as determinable by RT-PCR; (e) produce the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A; (f) secrete VEGF, HGF, IL-8, MCP-3, FGF2, Follistatin, G-CSF, EGF, ENA-78, GRO, IL-6, MCP-1, PDGF-BB, TIMP-2, uPAR, and Galectin-1 into culture medium in which the cell grows; (g) express micro RNAs miR-17-3p, miR-18a, miR-18b, miR-19b, miR-92, and miR-296 at a higher level than an equivalent number of bone marrow-derived mesenchymal stem cells; (h) express micro RNAs miR-20a, miR-20b, miR-221, miR-222, miR-15b, and miR-16 at a lower level than an equivalent number of bone marrow-derived mesenchymal stem cells; (i) express miRNAs miR-17-3p, miR-18a, miR-18b, miR-19b, miR-92, miR-20a, miR-20b, miR-296, miR-221, miR-222, miR-15b, and miR-16; or (j) express increased levels of CD202b, IL-8 and/or VEGF when cultured in less than about 5% O₂ compared to expression of CD202b, IL-8 and/or VEGF under 21% O₂.

In other embodiments, for example, the amnion derived adherent cells are adherent to tissue culture plastic, and are OCT-4⁻, as determinable by RT-PCR for 30 cycles, e.g., as compared to an appropriate control cell line, such as an embryonal carcinoma-derived stem cell line (e.g., NTERA-2, e.g., available from the American Type Culture Collection, ATCC Number CRL-1973). In a specific embodiment, the cells are OCT-4⁻, as determinable by RT-PCR, and VEGFR1/Flt-1⁺ (vascular endothelial growth factor receptor 1) and/or VEGFR2/KDR⁺ (vascular endothelial growth factor receptor 2, also known as kinase insert domain receptor), as determinable by immunolocalization, e.g., flow cytometry. In another specific embodiment, the cells are OCT-4⁻, as determinable by RT-PCR, and CD49f⁺ (integrin-α6⁺), as determinable by immunolocalization, e.g., flow cytometry. In a specific embodiment, said cells are OCT-4⁻, as determinable by RT-PCR, and HLA-G⁻, as determinable by RT-PCR. In another specific embodiment, said cells are OCT-4⁻, as determinable by RT-PCR, and CD90⁺, CD105⁺, or CD117⁻ as determinable by immunolocalization, e.g., flow cytometry. In a more specific embodiment, said OCT-4⁻ cells are CD90⁺, CD105⁺, and CD117⁻. In another specific embodiment, the cells are OCT-4⁻, and do not express SOX2, e.g., as determinable by RT-PCR for 30 cycles.

In another embodiment, said OCT-4⁻ cells are one or more of CD29⁺, CD73⁺, ABC-p⁺, and CD38⁻, as determinable by immunolocalization, e.g., flow cytometry.

In another specific embodiment, said OCT-4⁻ amnion derived adherent cells are additionally one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻ (angiotensin-I-converting enzyme, ACE), CD146⁻ (melanoma cell adhesion molecule), CXCR4⁻ (chemokine (C—X—C motif) receptor 4) as determinable by immunolocalization, e.g., flow cytometry. In a more specific embodiment, said amnion derived adherent cells are CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry. In another more specific embodiment, the amnion derived adherent cells provided herein are OCT-4⁻, as determinable by RT-PCR; VEGFR1/Flt-1⁺ and/or VEGFR2/KDR⁺, as determinable by immunolocalization, e.g., flow cytometry; and one or more, or all, of CD31⁻, CD34⁻, CD45⁻, CD133⁻, and/or Tie-2⁻ as determinable by immunolocalization, e.g., flow cytometry. In a specific embodiment, the amnion derived adherent cells express at least 2 log less PCR-amplified mRNA for OCT-4 at, e.g., >20 cycles, such as 20-30 cycles, than an equivalent number of NTERA-2 cells. In another specific embodiment, said OCT-4⁻ cells are additionally VE-cadherin⁻ (CD144⁻) as determinable by immunolocalization, e.g., flow cytometry. In another specific embodiment, said OCT-4⁻ cells are additionally positive for CD105⁺ and CD200⁺ as determinable by immunolocalization, e.g., flow cytometry. In another specific embodiment, said OCT-4⁻ cells do not express CD34, e.g., as detected by immunolocalization (e.g., flow cytometry), after exposure to 1 to 100 ng/mL VEGF (vascular endothelial growth factor) for 4 to 21 days.

In another embodiment, the amnion derived adherent cells are adherent to tissue culture plastic, and are OCT-4⁻ and SOX-2⁻, as determinable by RT-PCR. In yet another embodiment, said cells are CD90⁺, CD105⁺, and CD117⁻, as determinable by flow cytometry. In a specific embodiments, the OCT-4⁻, SOX-2⁻ amnion derived adherent cells are additionally HLA-G⁻ or CD271⁻, as determinable by flow cytometry. In a more specific embodiment, said cells are OCT-4⁻ and SOX-2⁻, as determinable by RT-PCR; and CD90⁺, CD105⁺, CD117⁻, CD271⁻ and HLA-G⁻, as determinable by flow cytometry.

In another embodiment of, and in addition to, any of the above AMDACs, said cell is adherent to tissue culture plastic, and positive for VEGFR2/KDR⁺ (CD309).

The amnion derived adherent cells disclosed herein, in another embodiment, are adherent to tissue culture plastic, are OCT-4⁻, as determinable by RT-PCR at, e.g. >20 cycles, such as 20-30 cycles, and are one or more of VEGFR2/KDR⁺, CD9⁺, CD54⁺, CD105⁺, CD200⁺, or VE-cadherin⁻, as determinable by immunolocalization, e.g., flow cytometry. In a specific embodiment, said cells are OCT-4⁻, as determinable by RT-PCR at, e.g., >20 cycles, such as 20-30 cycles, and VEGFR2/KDR⁺, CD9⁺, CD54⁺, CD105⁺, CD200⁺, and VE-cadherin⁻, as determinable by immunolocalization, e.g., flow cytometry. In another specific embodiment, the cells do not express CD34, e.g., as detected by immunolocalization (e.g., flow cytometry), after exposure to 1 to 100 ng/mL VEGF for 4 to 21 days.

In another embodiment, the amnion derived adherent cells are OCT-4⁻, CD49f⁺, HLA-G⁻, CD90⁺, CD105⁺, and CD117⁻. In a more specific embodiment, said cells are one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻ (melanoma cell adhesion molecule), or CXCR4⁻, as determinable by immunolocalization, e.g., flow cytometry. In a more specific embodiment, said cells are CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry. In another specific embodiment, said cells are VEGFR1/Flt-1⁺ and/or VEGFR2/KDR⁺, as determinable by immunolocalization, e.g., flow cytometry; and one or more of CD31⁻, CD34⁻, CD45⁻, CD133⁻, and/or Tie-2⁺ as determinable by immunolocalization, e.g., flow cytometry. In another specific embodiment, said cell is additionally VEGFR1/Flt-1⁺, VEGFR2/KDR⁺, CD31⁻, CD34⁻, CD45⁻, CD133⁻, and Tie-2⁺ as determinable by immunolocalization, e.g., flow cytometry.

In another embodiment, the amnion derived adherent cells disclosed herein do not express mRNA for one or more of ANGPT4, ANGPTL3, BGLAP, CD31, CD34, CDH5, CXCL10, DLX5, FGA, FGF4, FLT3, HLA-G, IFNG, LECT1, LEP, MMP-13, NANOG, Nestin, PLG, POU5F1, PRL, PROK1, SOX2, TERT, TNMD, and/or XLKD1 as determinable by RT-PCR, e.g., for 30 cycles. In another embodiment, the amnion derived adherent cells do not constitutively express one or more of invariant chain, HLA-DR-DP-DQ, CD6, or CD271, as determinable by flow cytometry, that is, the amnion derived adherent cells do not generally express these markers under normal, unstimulated conditions.

In a specific embodiment, the AMDACs described herein are telomerase⁻, as measured by RT-PCR and/or telomeric repeat amplification protocol (TRAP) assays. In another specific embodiment, the AMDACs described herein do not express mRNA for telomerase reverse transcriptase (TERT) as determinable by RT-PCR, e.g., for 30 cycles. In another specific embodiment, the AMDACs described herein are NANOG⁻, as measured by RT-PCR. In another specific embodiment, the AMDACs described herein do not express mRNA for NANOG as determinable by RT-PCR, e.g., for 30 cycles. In a specific embodiment, the AMDACs described herein are (sex determining region Y)-box 2 (SOX2)⁻. In another specific embodiment, the AMDACs described herein do not express mRNA for SOX2 as determinable by RT-PCR, e.g., for 30 cycles.

Further provided herein is an isolated population of cells comprising amnion derived adherent cells, wherein the population of cells is therapeutically effective in the methods of treatment disclosed herein. Such populations of cells can comprise any of the amnion derived adherent cells, described by any of the combinations of markers, as disclosed herein. In specific embodiments, at least about 50%, 60%, 70%, 80%, 90%, 95%, 98% or 99% of cells in said population are such amnion derived adherent cells. In other specific embodiments, at least 25%, 35%, 45%, 50%, 60%, 75%, 85% or more of the cells in the isolated population of cells comprising amnion derived adherent cells are not OCT-4⁺.

In certain embodiments, the methods of treatment provided herein comprise additionally administering a second type of cell to said individual. In specific embodiment, the isolated population of amnion derived adherent cells additionally comprises a second type of cell, e.g., stem cells or progenitor cells. In specific embodiments, the AMDACs disclosed herein comprise at least 10%, 15%, 20%, 25%, 30%, 35%, 40%, 45%, 50%, 55%, 60%, 65%, 70%, 80%, 85%, 90%, 95% or at least 98% of cells in said population. In other specific embodiments, at least 25%, 35%, 45%, 50%, 60%, 75%, 85% or more of the cells in the population of cells comprising amnion derived adherent cells and a second type of cell are not OCT-4⁺. In a specific embodiment, the second type of cells are contained within or isolated from placental blood, umbilical cord blood, crude bone marrow or other tissues. In a more specific embodiment, said second type of cells are embryonic stem cells, stem cells isolated from peripheral blood, stem cells isolated from placental blood, stem cells isolated from placental perfusate, stem cells isolated from placental tissue, stem cells isolated from umbilical cord blood, umbilical cord stem cell (e.g., stem cells from umbilical cord matrix or Wharton's jelly), bone marrow-derived mesenchymal stem cells, mesenchymal stromal cells, hematopoietic stem cells or progenitor cells, e.g., CD34⁺ cells, somatic stem cell, adipose stem cells, induced pluripotent stem cells, or the like. In another more specific embodiment, said second type of cells comprise at least 10%, 15%, 20%, 25%, 30%, 35%, 40%, 45%, or 50% of cells in said population.

In another specific embodiment, any of the above AMDACs, or second type of cells, are, or have been, proliferated in culture. In another specific embodiment, any of the above cells are from a culture of such cells that has been passaged at least 1, 2, 3, 4, 5, 6, 7, 8, 9, 10, 11, 12, 13, 14, 15, 16, 17, 18, 19, or 20 times, or more. In another specific embodiment, any of the above cells are from a culture of such cells that has doubled at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

In other embodiments, the methods of treatment disclosed herein comprise administering the AMDACs to an affected individual, in a composition, e.g., a pharmaceutical composition. In specific embodiments, the composition is a matrix or scaffold, e.g., a natural tissue matrix or scaffold, for example, a permanent or degradable decellularized tissue matrix or scaffold; or synthetic matrix or scaffold. In a more specific embodiment, said matrix or scaffold is shaped in the form of a bead, tube or other three-dimensional form. In another more specific embodiment, said matrix is a decellularized tissue matrix. In another specific embodiment, the composition comprises one or more of the isolated amnion derived adherent cells provided herein, or population of cells comprising the amnion derived adherent cells, in a physiologically-acceptable solution, e.g., a saline solution, culture medium or the like.

In another specific embodiment of the methods of treatment provided herein, said cells are administered to said individual by injection. In another specific embodiment, said cells are administered to said individual by intravenous infusion. In another specific embodiment of the method of treatment, said cells are administered to said individual by implantation in said individual of a matrix or scaffold comprising amnion derived adherent cells, as described above.

The isolated amnion derived adherent cells and cell populations provided herein are not the isolated placental stem cells or cell populations described, e.g., in U.S. Pat. No. 7,255,879 or U.S. Patent Application Publication No. 2007/0275362. The isolated amnion derived adherent cells provided herein are also not endothelial progenitor cells, amniotic epithelial cells, trophoblasts, cytotrophoblasts, embryonic germ cells, embryonic stem cells, cells obtained from the inner cell mass of an embryo, or cells obtained from the gonadal ridge of an embryo.

As used herein, the term “about” means, e.g., within 10% of a stated figure or value.

As used herein, the term “stem cell” defines the functional properties of any given cell population that can proliferate extensively, e.g., up to about 40 population doublings, but not necessarily infinitely, and can differentiate, e.g., differentiate in vitro, into multiple cell types.

As used herein, the term “progenitor cell” defines the functional properties of any given cell population that can proliferate extensively, e.g., up to about 40 population doublings, but not necessarily infinitely, and can differentiate, e.g., differentiate in vitro, into a restricted set of cell types, which is generally more restricted in comparison to that of a stem cell.

As used herein, the term “derived” means isolated from or otherwise purified. For example, amnion derived adherent cells are isolated from amnion. The term “derived” encompasses cells that are cultured from cells isolated directly from a tissue, e.g., the amnion, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “isolated cell” means a cell that is substantially separated from other, cells of the tissue, e.g., amnion, from which the cell is derived. A cell is “isolated” if at least about 20%, 30%, 40%, 50%, 60%, 70%, 80%, 90%, 95%, or at least about 99% of the cells with which the stem cell is naturally associated are removed from the cell, e.g., during collection and/or culture of the cell.

As used herein, the term “isolated population of cells” means a population of cells that is substantially separated from other cells of the tissue, e.g., amnion or placenta, from which the population of cells is derived. A population of cells is “isolated” if at least about 20%, 30%, 40%, 50%, 60%, 70%, 80%, 90%, 95%, or at least 99% of the cells with which the population of cells, or cells from which the population of cells is derived, is naturally associated are removed from the cell, e.g., during collection and/or culture of amnion derived adherent cells.

As used herein, a cell is “positive” for a particular marker when that marker is detectable above background, e.g., by immunolocalization, e.g., by flow cytometry; or by RT-PCR. For example, a cell is described as positive for, e.g., CD105 if CD105 is detectable on the cell in an amount detectably greater than background (in comparison to, e.g., an isotype control). In the context of, e.g., antibody-mediated detection, “positive,” as an indication a particular cell surface marker is present, means that the marker is detectable using an antibody, e.g., a fluorescently-labeled antibody, specific for that marker; “positive” also means that a cell bears that marker in a amount that produces a signal, e.g., in a cytometer, that is detectably above background. For example, a cell is “CD105⁺” where the cell is detectably labeled with an antibody specific to CD105, and the signal from the antibody is detectably higher than a control (e.g., background). Conversely, “negative” in the same context means that the cell surface marker is not detectable using an antibody specific for that marker compared to background. For example, a cell is “CD34⁻” where the cell is not detectably labeled with an antibody specific to CD34. Unless otherwise noted herein, cluster of differentiation (“CD”) markers are detected using antibodies. For example, OCT-4 can be determined to be present, and a cell is OCT-4⁺, if mRNA for OCT-4 is detectable using RT-PCR, e.g., for 30 cycles.

### 4. BRIEF DESCRIPTION OF THE FIGURES

FIG. 1 shows expression of stem cell-related genes by amnion derived adherent cells and NTERA-2 cells.

FIG. 2 shows the expression of TEM-7 on the cell surface of amnion derived adherent cells (AMDACs).

FIGS. 3A-3D show the secretion of selected angiogenic proteins by amnion derived adherent cells. FIG. 3A: Secretion by passage six AMDACs (n=3) of tissue inhibitor of metalloprotease-1 (TIMP-1), TIMP-2, thrombopoietin, vascular endothelial growth factor (VEGF), and VEGF-D. FIG. 3B: Secretion by passage six AMDACs (n=3) of angiogenin, epidermal growth factor (EGF), epithelial neutrophil-activating peptide 78 (ENA-78), basic fibroblast growth factor (bFGF), and growth-regulated oncogene alpha (GRO). FIG. 3C: Secretion by passage six AMDACs (n=3) of interferon gamma (IFN-gamma), insulin-like growth factor-1 (IGF-1), interleukin-6 (IL-6), IL-8, and leptin. FIG. 3D: Secretion by passage six AMDACs (n=3) of monocyte chemotactic protein-1 (MCP-1), platelet-derived growth factor (PDGF)-BB, placental growth factor (PIGF), rantes, and transforming growth factor-beta (TGF-beta).

FIG. 4 demonstrates the ability of AMDACs to inhibit T cell proliferation in vitro. NHDF: neonatal human dermal fibroblasts. Bars to left for AMDAC, NHDF: CD4+ T cell suppression compared to absence of AMDACs or NHDFs. Bars to right for AMDAC, NHDF: CD8+ T cell suppression compared to absence of AMDACs or NHDFs. Y axis: percent suppression attributable to AMDACs or NHDFs as compared to T cell proliferation in the absence of AMDACs or NHDFs.

FIG. 5 demonstrates that media conditioned by AMDACs induces suppression of TNF-alpha production by T cells. Y axis: percent suppression of production of TNF-α by bulk T cells in the presence of AMDACs or NHDFs as compared to production of TNF-α in the absence of AMDACs or NHDFs.

FIG. 6 shows suppression by AMDACs of Th1 T cells. Pan T base: percent of Th1 T cells in the absence of AMDACs. 100K, 75K, 50K, 25K: percent Th1 T cells in the presence of 100,000, 75,000, 50,000, and 25,000 AMDACs, respectively.

FIG. 7 shows suppression by AMDACs of Th17 T cells in a dose-dependent manner. 100K, 80K, 60K, 40K: percent Th17 T cells (in the absence of AMDACs) remaining after coculture with 100,000, 80,000, 60,000, and 40,000 AMDACs, respectively.

FIG. 8 shows increase of FoxP3 Treg cells by AMDACs. Baseline: percent of FoxP3 Treg cells in total T cells in the absence of AMDACs. 100K, 75K, 50K, 25K: percent FoxP3 Treg cells in the presence of 100,000, 75,000, 50,000, and 25,000 AMDACs, respectively.

FIGS. 9A-9C depict flow cytometry results of DC populations as assessed by CD86 and HLA-DR expression. All: SSC: side scatter gate. Cell type: dendritic cells (DC) alone, or DC+AMDACs. LPS+IFN-γ: cells stimulated (+) or not stimulated (−) with bacterial lipopolysaccharide and interferon gamma. FIG. 9A: DC labeled with anti-CD86-phycoerythrin (PE). FIG. 9B: DC labeled with anti-HLA-DR-PerCP Cy5.5. FIG. 9C: DC labeled with anti-IL-12-PE (Y-axis) and anti-CD11c-FITC.

FIG. 10 depicts suppression of production of tumor necrosis factor-alpha (TNF-α) and interleukin-12 (IL-12 by bacterial lipopolysaccharide (LPS)-stimulated dendritic cells (DCs). For each condition (IL-12 or TNF-α production), the left column is the production of the cytokine by DCs in the presence of LPS and interferon-gamma (IFN-γ), and the right column is the production of the cytokine by DCs in the presence of LPS, IFN-γ, and AMDACs. “□-” indicates condition in which DCs were not stimulated with either LPS or IFN-γ. Numbers to the right of each condition indicate the number of picograms of IL-12 or TNF-α produced by DC in each condition.

FIG. 11 depicts AMDAC-mediated suppression of natural killer (NK) cell proliferation. X axis: number of days of culture of NK cell precursors with (left bars) or without (right bars) AMDACs. Y axis: number of NK cells at each day of culture indicated.

FIG. 12 depicts AMDAC suppression of NK cell cytotoxicity. X axis: number of AMDACs per well in a coculture with NK cells and K562 cells (a human immortalized myelogenous leukemia cell line) as targets. Y axis: Percent NK cytotoxicity, calculated as (1−total number of K562 cells in the sample÷total K562 cells in a control containing no NK cells)×100.

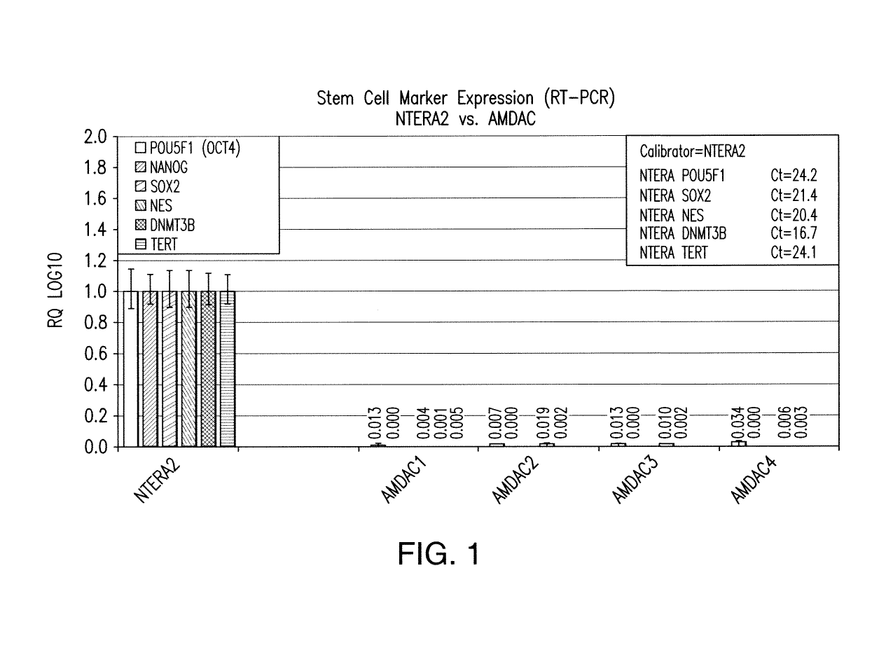

### 5. DETAILED DESCRIPTION

5.1 Methods of Treating a CNS Injury

Provided herein are methods for the treatment of an individual having an injury to the CNS, e.g., a spinal cord injury or traumatic brain injury, comprising administering to the individual having the CNS injury one or more doses of amnion derived adherent cells (“AMDACs”). Methods for the treatment of such individuals, and for the administration of AMDACs, alone or in combination with other therapies, are discussed in detail below.

5.1.1 Treatment of Spinal Cord Injury (SCI)

Provided herein are methods of treating an individual having, or experiencing, a symptom of or a condition or syndrome related to, a spinal cord injury (SCI), comprising administering to the individual a therapeutically effective amount of AMDACs, or medium conditioned by AMDACs, wherein the therapeutically effective amount is an amount sufficient to cause a detectable improvement in one or more symptoms of, or a reduction in the progression of one or more symptoms of, said spinal cord injury. As used herein, “one or more symptoms” includes objectively measurable parameters, such as degree of inflammation, immune response, gene expression within the site of injury that is correlated with the healing process, quality and extent of scarring at the site of injury, improvement in the patient's motor and sensory function, etc., and subjectively measurable parameters, such as patient well-being, patient perception of improvement in motor and sensory function, perception of lessening of pain or discomfort associated with the SCI, and the like.

Spinal cord injury is an insult to the spinal cord resulting in a change, either temporary or permanent, in its normal motor, sensory, or autonomic function. SCI includes conditions known as tetraplegia (formerly known as quadriplegia) and paraplegia. Thus, in some embodiments of the method of treatment of SCI provided herein, the individual having, or experiencing, a symptom of or a condition or syndrome related to, a spinal cord injury is tetraplegic or paraplegic.

Tetraplegia refers to injury to the spinal cord in the cervical region, characterized by impairment or loss of motor and/or sensory function in the cervical segments of the spinal cord due to damage of neural elements within the spinal canal. Tetraplegia results in impairment of function in the arms as well as in the trunk, legs and pelvic organs. It does not include brachial plexus lesions or injury to peripheral nerves outside the neural canal.

Paraplegia refers to impairment or loss of motor and/or sensory function in the thoracic, lumbar or sacral (but not cervical) segments of the spinal cord, secondary to damage of neural elements within the spinal canal. With paraplegia, arm functioning is spared, but, depending on the level of injury, the trunk, legs and pelvic organs may be involved. The term is used in referring to cauda equina and conus medullaris injuries, but not to lumbosacral plexus lesions or injury to peripheral nerves outside the neural canal.

Common causes of SCI include, but are not limited to, motor vehicle accidents, falls, violence, sports injuries, vascular disorders, tumors, infectious conditions, spondylosis, latrogenic injuries (especially after spinal injections and epidural catheter placement), vertebral fractures secondary to osteoporosis, and developmental disorders.

In certain embodiments, the spinal cord injury can result from, e.g., blunt force trauma, compression, displacement, or the like. In certain embodiments, the spinal cord is completely severed. In certain other embodiments, the spinal cord is damaged, e.g., partially severed, but not completely severed. In other embodiments, the spinal cord is compressed, e.g., through damage to the bony structure of the spinal column, displacement of one or more vertebrae relative to other vertebrae, inflammation or swelling of adjacent tissues, or the like.

In one embodiment, the spinal cord injury is at one or more of the cervical vertebrae. In another embodiment, the spinal cord injury is at one or more of the thoracic vertebrae. In another embodiment, the spinal cord injury is at one or more of the lumbar vertebrae. In another embodiment, the spinal cord injury is at one or more of the sacral vertebrae. In certain embodiments, the spinal cord injury is at vertebra C1, C2, C3, C4, C5, C6 or C7; or at vertebra T1, T2, T3, T4, T5, T6, T7, T8, T9, T10, T11 or T12; or at vertebra L1, L2, L3, L4 or L5. In certain other embodiments, the spinal cord injury is to a spinal root exiting the spinal column between C1 and C2; between C2 and C3; Between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5. In certain embodiments, the injury is to the cervical cord. In other embodiments, the injury is to the thoracic cord. In other embodiments the spinal cord injury is to the lumbrosacral cord. In certain other embodiments, the spinal cord injury is to the conus. In certain other embodiments, the CNS injury is to one or more nerves in the cauda equina. In another embodiment, the spinal cord injury is at the occiput.

In certain embodiments, a symptom of a spinal cord injury is numbness in one or more dermatomes (i.e., a patch of skin innervated by a given spinal cord level). In specific embodiments, the symptom of a spinal cord injury is numbness in one or more of dermatomes C1, C2, C3, C4, C5, C6, C7, T1, T2, T3, T4, T5, T6, T7, T8, T9, T10, T11, T12, L1, L2, L3, L4 or L5.

Spinal shock is a state of transient physiologic (rather than anatomic) reflex depression of cord function below the level of injury, with associated loss of all sensorimotor functions. An initial increase in blood pressure due to the release of catecholamines, followed by hypotension, is noted. Flaccid paralysis, including of the bowel and bladder, is observed, and sometimes sustained priapism develops. These symptoms tend to last several hours to days until the reflex arcs below the level of the injury begin to function again (e.g., bulbocavernosus reflex, muscle stretch reflex [MSR]). Therefore, in specific embodiments of the method, the therapeutically effective amount of AMDACs is an amount sufficient to cause a detectable improvement in one or more symptoms of spinal shock resulting from SCI, including, but not limited to, loss of some or all sensorimotor function, high blood pressure, hypotension, flaccid paralysis (e.g., of the bowel and bladder), and priapism.

Neurogenic shock is manifested by the triad of hypotension, bradycardia, and hypothermia. Shock tends to occur more commonly in injuries above T6, secondary to the disruption of the sympathetic outflow from T1-L2 and to unopposed vagal tone, leading to a decrease in vascular resistance, with associated vascular dilatation. Neurogenic shock is distinct from spinal and hypovolemic shock, which tends to be associated with tachycardia. Thus, in some embodiments of the method of treating SCI, the therapeutically effective amount of AMDACs is an amount sufficient to cause a detectable improvement in one or more symptoms of neurogenic shock resulting from SCI, including, but not limited to, hypotension, bradycardia, hypothermia, a decrease in vascular resistance, and vascular dilatation.

Autonomic dysreflexia (AD) is a syndrome of massive imbalanced reflex sympathetic discharge occurring in patients with SCI above the splanchnic sympathetic outflow (T5-T6). AD occurs after the phase of spinal shock in which reflexes return. Individuals with injury above the major splanchnic outflow may develop AD. Below the injury, intact peripheral sensory nerves transmit impulses that ascend in the spinothalamic and posterior columns to stimulate sympathetic neurons located in the intermediolateral gray matter of the spinal cord. The inhibitory outflow above the SCI from cerebral vasomotor centers is increased, but it is unable to pass below the block of the SCI. This large sympathetic outflow causes release of various neurotransmitters (norepinephrine, dopamine-b-hydroxylase, dopamine), causing piloerection, skin pallor, and severe vasoconstriction in arterial vasculature. The result is sudden elevation in blood pressure and vasodilation above the level of injury. Patients commonly have a headache caused by vasodilation of pain sensitive intracranial vessels. Thus, in some embodiments of the method of treating SCI, the therapeutically effective amount of AMDACs is an amount sufficient to cause a detectable improvement in one or more symptoms of autonomic dysreflexia resulting from SCI, including, but not limited to, piloerection, skin pallor, severe vasoconstriction in arterial vasculature, elevation in blood pressure, and vasodilation above the level of injury.

In some embodiments of the method of treating SCI, the therapeutically effective amount of AMDACs is an amount sufficient to cause a detectable improvement in one or more symptoms of edema resulting from SCI. In some embodiments of the method, the therapeutically effective amount of AMDACs is an amount sufficient to cause a detectable improvement in one or more symptoms of SCI caused by destruction from direct trauma. In some embodiments of the method, the therapeutically effective amount of AMDACs is an amount sufficient to cause a detectable improvement in one or more symptoms of SCI caused by compression by bone fragments. In some embodiments of the method, the therapeutically effective amount of AMDACs is an amount sufficient to cause a detectable improvement in one or more symptoms of SCI caused by compression of disc material.

The methods of treating SCI provided herein also provide for the treatment of an individual having, or experiencing, a symptom of or a condition or syndrome related to other classifications of SCI including, but not limited to, central cord syndrome, Brown-Séquard syndrome, anterior cord syndrome, conus medullaris syndrome, and cauda equina syndrome.

Central cord syndrome often is associated with a cervical region injury and leads to greater weakness in the upper limbs than in the lower limbs, with sacral sensory sparing. Thus, in specific embodiments of the method of treating SCI, the therapeutically effective amount of AMDACs is an amount sufficient to cause a detectable improvement in one or more symptoms of central cord syndrome, including, but not limited to, greater weakness in the upper limbs than in the lower limbs, with sacral sensory sparing.

Brown-Séquard syndrome, which often is associated with a hemisection lesion of the cord, causes a relatively greater ipsilateral proprioceptive and motor loss, with contralateral loss of sensitivity to pain and temperature. Thus, in specific embodiments of the method of treating SCI, the therapeutically effective amount of AMDACs is an amount sufficient to cause a detectable improvement in one or more symptoms of Brown-Séquard syndrome, including, but not limited to, ipsilateral proprioceptive and motor loss, with contralateral loss of sensitivity to pain and temperature.

Anterior cord syndrome often is associated with a lesion causing variable loss of motor function and sensitivity to pain and temperature; proprioception is preserved. Thus, in specific embodiments of the method of treating SCI, the therapeutically effective amount of AMDACs is an amount sufficient to cause a detectable improvement in one or more symptoms of anterior cord syndrome, including, but not limited to, variable loss of motor function and sensitivity to pain and temperature.

Conus medullaris syndrome is associated with injury to the sacral cord and lumbar nerve roots leading to areflexic bladder, bowel, and lower limbs, while the sacral segments occasionally may show preserved reflexes (e.g., bulbocavernosus and micturition reflexes). Thus, in specific embodiments of the method of treating SCI, the therapeutically effective amount of AMDACs is an amount sufficient to cause a detectable improvement in one or more symptoms of conus medullaris syndrome, including, but not limited to, areflexic bladder, bowel, and lower limbs.

Cauda equina syndrome is due to injury to the lumbosacral nerve roots in the spinal canal, leading to areflexic bladder, bowel, and lower limbs. Thus, in specific embodiments of the method of treating SCI, the therapeutically effective amount of AMDACs is an amount sufficient to cause a detectable improvement in one or more symptoms of cauda equina syndrome, including, but not limited to, areflexic bladder, bowel, and lower limbs.

In certain embodiments, the particular technique(s) for detecting an improvement in, a reduction in the severity of, or a reduction in the progression of, one or more symptoms, conditions, or syndromes of SCI is not critical to the method of treating SCI provided herein. In certain embodiments, the assessment of said improvement or reduction in the progression of one or more symptoms, conditions, or syndromes of SCI is determined according to the judgment of the practitioner in the art. In certain embodiments, the assessment of said improvement or reduction in the progression of one or more symptoms, conditions, or syndromes of SCI is determined according to the judgment of the practitioner in the art in combination with the subjective experience of the subject.

In some embodiments, an improvement in one or more symptoms of, or a reduction in the progression of one or more symptoms of, said spinal cord injury is detected in accordance with the International Standards for Neurological and Functional Classification of Spinal Cord Injury. The International Standards for Neurological and Functional Classification of Spinal Cord Injury, published by the American Spinal Injury Association (ASIA), is a widely accepted system describing the level and extent of spinal cord injury based on a systematic motor and sensory examination of neurologic function. See International Standards For Neurological Classification Of Spinal Cord Injury, J Spinal Cord Med. 26 Suppl 1:S50-6 (2003), the disclosure of which is hereby incorporated by reference in its entirety.

In particular embodiments, an improvement in one or more symptoms of, or a reduction in the progression of one or more symptoms of, said spinal cord injury is detected in accordance with the ASIA Impairment Scale (modified from the Frankel classification), using the following categories:
A—Complete: No sensory or motor function is preserved in sacral segments S4-S5.4. “Complete” refers to the absence of sensory and motor functions in the lowest sacral segments.
B—Incomplete: Sensory, but not motor, function is preserved below the neurologic level and extends through sacral segments S4-S5. “Incomplete” refers to preservation of sensory or motor function below the level of injury, including the lowest sacral segments.
C—Incomplete: Motor function is preserved below the neurologic level, and most key muscles below the neurologic level have muscle grade less than 3.
D—Incomplete: Motor function is preserved below the neurologic level, and most key muscles below the neurologic level have muscle grade greater than or equal to 3.
E—Normal: Sensory and motor functions are normal.

Thus, in a specific embodiment of the method of treating SCI provided herein, the therapeutically effective amount of AMDACs is an amount sufficient to cause a decrease in impairment according to the ASIA impairment scale (AIS). In some embodiments, the decrease is a one, two, three, four or five grade reduction in impairment, wherein one grade corresponds to a single category improvement, for example, a reduction in impairment from category D to category E. In some embodiments, the therapeutically effective amount of AMDACs is an amount sufficient to convert an individual classified as ASIA A to ASIA B, ASIA C, ASIA D or ASIA E according to the AIS. In some embodiments, the therapeutically effective amount of AMDACs is an amount sufficient to convert an individual classified as ASIA B to ASIA C, ASIA D or ASIA E according to the AIS. In some embodiments, the therapeutically effective amount of AMDACs is an amount sufficient to convert an individual classified as ASIA C to ASIA D or ASIA E according to the AIS. In some embodiments, the therapeutically effective amount of AMDACs is an amount sufficient to convert an individual classified as ASIA D to ASIA E according to the AIS.

In some embodiments, an improvement in one or more symptoms of, or a reduction in the progression of one or more symptoms of said spinal cord injury is detected by measuring the muscle strength of the patient. In some embodiments, muscle strength can be graded using the following Medical Research Council (MRC) scale of 0-5:

5—Normal power

4+—Submaximal movement against resistance

4—Moderate movement against resistance

4⁻—Slight movement against resistance

3—Movement against gravity but not against resistance

2—Movement with gravity eliminated

1—Flicker of movement

0—No movement

The following key muscles are tested in patients with SCI, and the corresponding level of injury is indicated:

C5—Elbow flexors (biceps, brachialis)

C6—Wrist extensors (extensor carpi radialis longus and brevis)

C7—Elbow extensors (triceps)

C8—Finger flexors (flexor digitorum profundus) to the middle finger

T1—Small finger abductors (abductor digiti minimi)

L2—Hip flexors (iliopsoas)

L3—Knee extensors (quadriceps)

L4—Ankle dorsiflexors (tibialis anterior)

L5—Long toe extensors (extensors hallucis longus)

S1—Ankle plantar flexors (gastrocnemius, soleus)

Thus, in a specific embodiment of the method of treating SCI provided herein, the therapeutically effective amount of AMDACs is an amount sufficient to cause a one, two, three, four or five point increase in muscle strength according to the MRC scale. For example, in some embodiments, the therapeutically effective amount of AMDACs is an amount sufficient to cause a muscle having no movement as a result of the SCI to have a flicker of movement, movement with gravity eliminated, movement against gravity but not against resistance, slight movement against resistance, moderate movement against resistance, submaximal movement against resistance, or normal power. In some embodiments, the therapeutically effective amount of AMDACs is an amount sufficient to cause a muscle having only a flicker of movement as a result of the SCI to have movement with gravity eliminated, movement against gravity but not against resistance, slight movement against resistance, moderate movement against resistance, submaximal movement against resistance, or normal power. In some embodiments, the therapeutically effective amount of AMDACs is an amount sufficient to cause a muscle having only movement with gravity eliminated as a result of the SCI to have movement against gravity but not against resistance, slight movement against resistance, moderate movement against resistance, submaximal movement against resistance, or normal power. In some embodiments, the therapeutically effective amount of AMDACs is an amount sufficient to cause a muscle having only movement against gravity but not against resistance as a result of the SCI to have slight movement against resistance, moderate movement against resistance, submaximal movement against resistance, or normal power. In some embodiments, the therapeutically effective amount of AMDACs is an amount sufficient to cause a muscle having only slight movement against resistance as a result of the SCI to have moderate movement against resistance, submaximal movement against resistance, or normal power. In some embodiments, the therapeutically effective amount of AMDACs is an amount sufficient to cause a muscle having only moderate movement against resistance as a result of the SCI to have submaximal movement against resistance or normal power. In some embodiments, the therapeutically effective amount of AMDACs is an amount sufficient to cause a muscle having only submaximal movement against resistance as a result of the SCI to have normal power.

In some embodiments, the therapeutically effective amount of AMDACs is an amount sufficient to cause a one, two, three, four or five point increase in the strength of a biceps muscle of the subject. In some embodiments, the therapeutically effective amount of AMDACs is an amount sufficient to cause a one, two, three, four or five point increase in the strength of a brachialis muscle of the subject. In some embodiments, the therapeutically effective amount of AMDACs is an amount sufficient to cause a one, two, three, four or five point increase in the strength of a extensor carpi radialis longus or brevis muscle of the subject. In some embodiments, the therapeutically effective amount of AMDACs is an amount sufficient to cause a one, two, three, four or five point increase in the strength of a triceps muscle of the subject. In some embodiments, the therapeutically effective amount of AMDACs is an amount sufficient to cause a one, two, three, four or five point increase in the strength of a flexor digitorum profundus muscle of the subject. In some embodiments, the therapeutically effective amount of AMDACs is an amount sufficient to cause a one, two, three, four or five point increase in the strength of a abductor digiti minimi muscle of the subject. In some embodiments, the therapeutically effective amount of AMDACs is an amount sufficient to cause a one, two, three, four or five point increase in the strength of a iliopsoas muscle of the subject. In some embodiments, the therapeutically effective amount of AMDACs is an amount sufficient to cause a one, two, three, four or five point increase in the strength of a quadriceps muscle of the subject. In some embodiments, the therapeutically effective amount of AMDACs is an amount sufficient to cause a one, two, three, four or five point increase in the strength of a tibialis anterior muscle of the subject. In some embodiments, the therapeutically effective amount of AMDACs is an amount sufficient to cause a one, two, three, four or five point increase in the strength of a extensors hallucis longus muscle of the subject. In some embodiments, the therapeutically effective amount of AMDACs is an amount sufficient to cause a one, two, three, four or five point increase in the strength of a gastrocnemius or soleus muscle of the subject.

In some embodiments, an improvement in one or more symptoms of, or a reduction in the progression of one or more symptoms of, said spinal cord injury is detected by sensory testing. Sensory testing can be performed at the following levels:

C2—Occipital protuberance

C3—Supraclavicular fossa

C4—Top of the acromioclavicular joint

C5—Lateral side of antecubital fossa

C6—Thumb

C7—Middle finger

C8—Little finger

T1—Medial side of antecubital fossa

T2—Apex of axilla

T3—Third intercostal space (IS)

T4—Fourth IS at nipple line

T5—Fifth IS (midway between T4 and T6)

T6—Sixth IS at the level of the xiphisternum

T7—Seventh IS (midway between T6 and T8)

T8—Eighth IS (midway between T6 and T10)

T9—Ninth IS (midway between T8 and T10)

T10—10th IS or umbilicus

T11—11th IS (midway between T10 and T12)

T12—Midpoint of inguinal ligament

L1—Half the distance between T12 and L2

L2—Midanterior thigh

L3—Medial femoral condyle

L4—Medial malleolus

L5—Dorsum of the foot at third metatarsophalangeal joint

S1—Lateral heel

S2—Popliteal fossa in the midline

S3—Ischial tuberosity

S4-5—Perianal area (taken as 1 level)

Sensory scoring is for light touch and pinprick, as follows:

0—Absent

1—Impaired or hyperesthesia

2—Intact

A score of zero is given if the patient cannot differentiate between the point of a sharp pin and the dull edge. Thus, in a specific embodiment of the method of treatment provided herein, the therapeutically effective amount of AMDACs is an amount sufficient to cause a one or two point increase in sensory scoring corresponding to one or more of C2, C3, C4, C5, C6, C7, C8, T1, T2, T3, T4, T5, T6, T7, T8, T9, T10, T11, T12, L1, L2, L3, L4, L5, S1, S2, S3, S4 and S5.

In some embodiments, an improvement in one or more symptoms of, or a reduction in the progression of one or more symptoms of, said spinal cord injury is detected by monitoring the daily life functionality of the patient. In some embodiments of the method of treatment of SCI provided herein, the therapeutically effective amount of AMDACs is an amount sufficient to effect a functional improvement in the daily-life activities of the patient. In some embodiments, the Functional Independence Measure (FIM) is used to assess functional improvement of the patient. The FIM focuses on six areas of functioning: self-care, sphincter control, mobility, locomotion, communication and social cognition. Within each area, two or more specific activities/items are evaluated, with a total of 18 items. For example, six activity items (eating, grooming, bathing, dressing-upper body, dressing-lower body, and toileting) comprise the self-care area. Each of the 18 items is evaluated in terms of independence of functioning, using a seven-point scale:

Independent (No Human Assistance is Required):

7=Complete independence: The activity is typically performed safely, without modification, assistive devices or aids, and within reasonable time.

6=Modified independence: The activity requires an assistive device and/or more than reasonable time and/or is not performed safely.

Dependent (Human Supervision or Physical Assistance is Required):

5=Supervision or setup: No physical assistance is needed, but cuing, coaxing or setup is required.

4=Minimal contact assistance: Subject requires no more than touching and expends 75% or more of the effort required in the activity.

3=Moderate assistance: Subject requires more than touching and expends 50±75% of the effort required in the activity.

2=Maximal assistance: Subject expends 25±50% of the effort required in the activity.

1=Total assistance: Subject expends 0±25% of the effort required in the activity.

Thus, the FIM total score (summed across all items) estimates the cost of disability in terms of safety issues and of dependence on others and on technological devices. The profile of area scores and item scores pinpoints the specific aspects of daily living that have been most affected by SCI. In some embodiments of the method of treating SCI provided herein, the therapeutically effective amount of AMDACs is an amount sufficient to cause a one, two, three, four, five or six point increase in functioning of the patient according to the FIM scale. In some embodiments, the therapeutically effective amount of AMDACs is an amount sufficient to cause a subject requiring total assistance as a result of the SCI to require only moderate assistance, only minimal contact assistance, only supervision or setup, or to have modified independence or complete independence. In some embodiments, the therapeutically effective amount of AMDACs is an amount sufficient to cause a subject requiring moderate assistance as a result of the SCI to require only minimal contact assistance, only supervision or setup, or to have modified independence or complete independence. In some embodiments, the therapeutically effective amount of AMDACs is an amount sufficient to cause a subject requiring minimal contact assistance as a result of the SCI to require only supervision or setup, or to have modified independence or complete independence. In some embodiments, the therapeutically effective amount of AMDACs is an amount sufficient to cause a subject requiring supervision or setup as a result of the SCI to have modified independence or complete independence. In some embodiments, the therapeutically effective amount of AMDACs is an amount sufficient to cause a subject having modified independence as a result of the SCI to have complete independence.

An individual having, or experiencing, a symptom of SCI, can be treated with a plurality of AMDACs, and, optionally, one or more therapeutic agents, at any time during the progression of the injury. For example, the individual can be treated immediately after injury, or within 1, 2, 3, 4, 5, 6 days of injury, or within 1, 2, 3, 4, 5, 6, 7, 8, 9, 10, 11, 12, 13, 13, 15, 16, 17, 18, 19, 20, 25, 30, 35, 40, 45, 50 days or more of injury, or within 1, 2, 3, 4, 5, 6, 7, 8, 9, 10 or more years after injury. The individual can be treated once, or multiple times during the clinical course of the injury. In a specific embodiment of the method of treatment, said AMDACs are administered to said individual within 21 days of development of one or more symptoms of a spinal cord injury. In another specific embodiment of the method of treatment, said AMDACs are administered to said individual within 14 days of development of one or more symptoms of a spinal cord injury. In another specific embodiment of the method of treatment, said AMDACs are administered to said individual within 7 days of development of one or more symptoms of a spinal cord injury. In another specific embodiment of the method of treatment, said AMDACs are administered to said individual within 48 hours of development of one or more symptoms of a spinal cord injury. In another specific embodiment, said AMDACs are administered to said individual within 24 hours of development of one or more symptoms of a spinal cord injury. In another specific embodiment, said AMDACs are administered to said individual within 12 hours of development of one or more symptoms of a spinal cord injury. In another specific embodiment, said AMDACs are administered to said individual within 3 hours of development of one or more symptoms of a spinal cord injury.

In certain embodiments of the invention, the individual is an animal, preferably a mammal, more preferably a non-human primate. In certain embodiments, the individual is a human patient. The individual can be a male or female subject. In certain embodiments, the subject is a non-human animal, such as, for instance, a cow, sheep, goat, horse, dog, cat, rabbit, rat or mouse.

The AMDACs useful in the treatment of SCI can be any of the AMDACs disclosed herein. In a specific embodiment, the AMDACs are OCT-4⁻ (negative for OCT-4, also known as POU5F1 or octamer binding protein 4). In another specific embodiment, the AMDACs are OCT-4⁻ and VEGFR1/Flt-1⁺ (vascular endothelial growth factor receptor 1) and/or VEGFR2/KDR⁺ (vascular endothelial growth factor receptor 2, also known as kinase insert domain receptor). In another specific embodiment, the AMDACs are OCT-4⁻ and CD49f⁺ (integrin-α6⁺). In another specific embodiment, the AMDACs are OCT-4⁻ and HLA-G⁻. In another specific embodiment, the AMDACs are OCT-4⁻ and CD90⁺, CD105⁺, or CD117⁻. In another specific embodiment, the AMDACs are OCT-4⁻, CD90⁺, CD105⁺, and CD117⁻. In another specific embodiment, the AMDACs are OCT-4⁻ and do not express SOX2. In another specific embodiment, the AMDACs are GFAP⁺. In another specific embodiment, the AMDACs are beta-tubulin III (Tuj1)⁺. In another specific embodiment, the AMDACs are OCT-4⁻, GFAP⁺, and beta-tubulin III (Tuj1)⁺. In another specific embodiment, the AMDACs useful in the treatment of SCI are OCT-4⁻, CD200⁺, CD105⁺, and CD49f⁺. In another specific embodiment, the AMDACs useful in the treatment of SCI are CD200⁺, CD105⁺, CD90⁺, and CD73⁺. In another specific embodiment, the AMDACs useful in the treatment of SCI are CD117⁻ and not selected using an antibody to CD117. In another specific embodiment, the AMDACs useful in the treatment of SCI are CD146⁻ and not selected using an antibody to CD146. In another specific embodiment, the AMDACs useful in the treatment of SCI are OCT-4⁻ and do not express CD34 following induction with VEGF. In another specific embodiment, the AMDACs useful in the treatment of SCI are neurogenic, as determinable by a short-term neural differentiation assay (see, e.g., Section 5.12.1, below). In another specific embodiment, the AMDACs useful in the treatment of SCI are non-chondrogenic as determinable by an in vitro chondrogenic potential assay (see, e.g., Section 5.12.3, below). In another specific embodiment, the AMDACs useful in the treatment of SCI are non-osteogenic as determinable by an osteogenic phenotype assay (see, e.g., Section 5.12.2, below).

In a specific embodiment, the AMDACs useful in the treatment of SCI are telomerase⁻, as measured by RT-PCR and/or TRAP assays. In another specific embodiment, the AMDACs useful in the treatment of SCI do not express mRNA for telomerase reverse transcriptase (TERT) as determinable by RT-PCR, e.g., for 30 cycles. In another specific embodiment, the AMDACs useful in the treatment of SCI are NANOG⁻, as measured by RT-PCR. In another specific embodiment, the AMDACs useful in the treatment of SCI do not express mRNA for NANOG as determinable by RT-PCR, e.g., for 30 cycles. In a specific embodiment, the AMDACs useful in the treatment of SCI are (sex determining region Y)-box 2 (SOX2)⁻. In another specific embodiment, the AMDACs useful in the treatment of SCI do not express mRNA for SOX2 as determinable by RT-PCR, e.g., for 30 cycles. In another specific embodiment, the AMDACs useful in the treatment of SCI are not osteogenic as measured by an osteogenic phenotype assay (see, e.g., Section 5.12.2, below). In another specific embodiment, the AMDACs useful in the treatment of SCI are not chondrogenic as measured by a chondrogenic potential assay (see, e.g., Section 5.12.3, below). In another specific embodiment, the AMDACs useful in the treatment of SCI are not osteogenic as measured by an osteogenic phenotype assay (see, e.g., Section 5.12.2, below) and are not chondrogenic as measured by a chondrogenic potential assay (see, e.g., Section 5.12.3, below).

In one embodiment, the individual is administered a dose of about 300 million AMDACs. Dosage, however, can vary according to the individual's physical characteristics, e.g., weight, and can range from 1 million to 10 billion AMDACs per dose, preferably between 10 million and 1 billion per dose, or between 100 million and 500 million AMDACs per dose. The administration is preferably intravenous, but can be by any medically-acceptable route for the administration of live cells, e.g., intravenous, intraarterial, intraperitoneal, intraventricular, intrasternal, intracranial, intramuscular, intrasynovial, intraocular, intravitreal (e.g., where there is an ocular involvement), intracerebral, intracerebroventricular (e.g., where there is a neurologic or brain involvement), intrathecal, intraosseous infusion, intravesical, transdermal, intracisternal, epidural, or subcutaneous administration. In specific embodiments, administration is by bolus injection or infusion directly into the site of the spinal cord injury, e.g., via lumbar puncture.

In one embodiment, the AMDACs are from a cell bank. In one embodiment, a dose of AMDACs is contained within a blood bag or similar bag, suitable for bolus injection or administration by catheter.

AMDACs, or medium conditioned by AMDACs, can be administered in a single dose, or in multiple doses. Where AMDACs are administered in multiple doses, the doses can be part of a therapeutic regimen designed to relieve one or more acute symptoms of SCI, or can be part of a long-term therapeutic regimen designed to lessen the severity of SCI.

The methods for treating SCI provided herein further encompass treating SCI by administering a therapeutically effective amount of AMDACs in conjunction with one or more therapies or treatments used in the course of treating SCI. The one or more additional therapies may be used prior to, concurrent with, or after administration of the AMDACs. In some embodiments, the one or more additional therapies comprise the application of therapeutic spinal traction. Therapeutic spinal traction uses manually or mechanically created forces to stretch and mobilize the spine, based on the application of a force (usually a weight) along the longitudinal axis of the spinal column. If the neck or cervical segments are fractured, traction may straighten out and decompress the vertebral column.

In other embodiments, the one or more additional therapies comprise surgical stabilization of the spine, e.g., through the insertion of rods and screws to properly align the vertebral column or fuse adjacent vertebrae to strengthen the vertebra, promote bone re-growth, and reduce the likelihood of further spinal cord injury in the future. In other embodiments, the one or more additional therapies comprise rehabilitation (e.g., repetitive voluntary movement training, strength training, and the like), which can promote the formation of new local CNS connections. In other embodiments, the one or more additional therapies comprise functional electrical stimulation (FES) of specific nerves or muscles, for example, FES of phrenic nerves to assist breathing; FES of sacral roots to promote bladder and bowel function; FES of limb muscles to improve arm or hand function, as well as standing or walking.

Also provided herein are methods for the treatment of an individual having, or experiencing, a symptom of, SCI, comprising administering to the individual a plurality of AMDACs sufficient to cause a detectable improvement in one or more symptoms, conditions, or syndromes of, or a reduction in the progression of one or more symptoms, conditions, or syndromes of, said spinal cord injury, and one or more therapeutic agents. In one embodiment, the therapeutic agent is corticosteroid. In other embodiments, the therapeutic agent is an anticoagulant, such as heparin. In other embodiments, the therapeutic agent is a neuroprotective agent. In some embodiments the neuroprotective agent is methylprednisolone sodium succinate (MPSS), GM-1 (Sygen), Gacylidine (GK-11), thyrotropin releasing hormone, monocycline (minocycline), lithium or erythropoietin (EPO).

In other embodiments the therapeutic agent or a Rho antagonist, e.g., Cethrinr, inosine, rolipram, ATI-355 (NOGO), chondroitinase, fampridine (4-aminopyrideine) or Gabapentin. In another embodiment, the therapeutic agent is an immunomodulatory or immunosuppressive agent, e.g., Cyclosporin A. In other embodiments, the therapeutic agent is a second population of cells that is co-administered with the AMDACs. In some embodiments, the second population of cells is a population of autologous macrophages, bone marrow stromal cells, nasal olfactory ensheathing cells, embryonic olfactory cortex cells, or Schwann cells.

5.1.2 Treatment of Traumatic Brain Injury (TBI)

Also provided herein are methods of treating an individual having, or experiencing, a symptom of a traumatic brain injury (TBI), comprising administering to the individual a therapeutically effective amount of AMDACs, or medium conditioned by AMDACs, wherein the therapeutically effective amount is an amount sufficient to cause a detectable improvement in one or more symptoms of, or a reduction in the progression of one or more symptoms of, said traumatic brain injury. As used herein, “one or more symptoms” includes objectively measurable parameters, such as degree of inflammation, immune response, gene expression within the site of injury that is correlated with the healing process, quality and extent of scarring at the site of injury, improvement in the patient's motor, sensory and cognitive function, etc., and subjectively measurable parameters, such as patient well-being, patient perception of improvement in motor, sensory and cognitive function, perception of lessening of pain or discomfort associated with the TBI, and the like.

TBI is a nondegenerative, noncongenital insult to the brain from an external mechanical force applied to the cranium and the intracranial contents, possibly leading to permanent or temporary impairment of cognitive, physical, and psychosocial functions, with an associated diminished or altered state of consciousness. TBI can manifest clinically from concussion to coma and death.

In some embodiments of the method of treating TBI, the therapeutically effective amount of AMDACs is an amount sufficient to cause a detectable improvement in one or more symptoms of a primary TBI, i.e., traumatic brain injury which occurs at the moment of trauma. In some embodiments, the primary TBI is a focal injury, e.g., a skull fracture, a laceration, a contusion, or a penetrating wound. In some embodiments, the primary TBI is diffuse, e.g., diffuse axonal injury.

In some embodiments of the method of treating TBI, the therapeutically effective amount of AMDACs is an amount sufficient to cause a detectable improvement in one or more symptoms of a secondary injury resulting from a primary TBI, which occurs immediately after trauma and produces effects that may continue for some period of time. Secondary types of TBI are attributable to further cellular damage from the effects of primary injuries. Secondary injuries may develop over a period of hours or days following the initial trauma to the brain.

The methods for treating TBI provided herein also encompass the treatment of TBI injuries inflicted upon specific areas to the brain. In some embodiments, the methods of treating TBI provided herein are useful for treating injuries to the frontal lobe (located at the forehead), parietal lobe (located near the back and top of the head), occipital lobe (located most posterior, at the back of the head), temporal lobes (located at the side of head above ears), brain stem (located deep within the brain) and the cerebellum (located at the base of the skull).

In a specific embodiment of the method of treating TBI provided herein, the therapeutically effective amount of AMDACs is an amount sufficient to cause an improvement in one or more symptoms of an injury to the frontal lobe, including, but not limited to, loss of simple movement of various body parts (paralysis), inability to plan a sequence of complex movements needed to complete multi-stepped tasks, such as making coffee (sequencing), loss of spontaneity in interacting with others, loss of flexibility in thinking, persistence of a single thought (perseveration), inability to focus on task (attending), mood changes (emotionally labile), changes in social behavior, changes in personality, difficulty with problem solving, or inability to express language (Broca's Aphasia).

In a specific embodiment of the method of treating TBI provided herein, the therapeutically effective amount of AMDACs is an amount sufficient to cause an improvement in one or more symptoms of an injury to the parietal lobe, including, but not limited to, an inability to attend to more than one object at a time, an inability to name an object (anomia), an inability to locate the words for writing (agraphia), problems with reading (alexia), difficulty with drawing objects, difficulty in distinguishing left from right, difficulty with doing mathematics (dyscalculia), lack of awareness of certain body parts and/or surrounding space (apraxia) that leads to difficulties in self-care, inability to focus visual attention, or difficulties with eye and hand coordination.

In a specific embodiment of the method of treating TBI provided herein, the therapeutically effective amount of AMDACs is an amount sufficient to cause an improvement in one or more symptoms of an injury to the occipital lobe, including, but not limited to, defects in vision (visual field cuts), difficulty with locating objects in environment, difficulty with identifying colors (color agnosia), production of hallucinations, visual illusions (inaccurately seeing objects), word blindness (inability to recognize words), difficulty in recognizing drawn objects, inability to recognize the movement of object (movement agnosia), or difficulties with reading and writing.

In a specific embodiment of the method of treating TBI provided herein, the therapeutically effective amount of AMDACs is an amount sufficient to cause an improvement in one or more symptoms of an injury to the temporal lobes including, but not limited to, difficulty in recognizing faces (prosopagnosia), difficulty in understanding spoken words (Wernicke's Aphasia), disturbance with selective attention to what the subject sees and hears, difficulty with identification of, and verbalization about objects, short term memory loss, interference with long term memory, increased and decreased interest in sexual behavior, inability to categorize objects (categorization), persistent talking (indicative of right lobe damage), or increased aggressive behavior.

In a specific embodiment of the method of treating TBI provided herein, the therapeutically effective amount of AMDACs is an amount sufficient to cause an improvement in one or more symptoms of an injury to the brain stem, including, but not limited to, decreased vital capacity in breathing (important for speech), difficulty with swallowing food and water (dysphagia), difficulty with organization/perception of the environment, problems with balance and movement, dizziness and nausea (vertigo), or sleeping difficulties (insomnia, sleep apnea).

In a specific embodiment of the method of treating TBI provided herein, the therapeutically effective amount of AMDACs is an amount sufficient to cause an improvement in one or more symptoms of an injury to the base of the skull, including, but not limited to, loss of ability to coordinate fine movements, loss of ability to walk, inability to reach out and grab objects, tremors, dizziness (vertigo), slurred speech (scanning speech), or inability to make rapid movements.

The methods for treating TBI provided herein also encompass the treatment of TBI injuries that range in scope from mild to severe. A traumatic brain injury (TBI) can be classified as mild if loss of consciousness and/or confusion and disorientation is shorter than 30 minutes. Thus, in some embodiments, the invention provides for the administration of an effective dose of AMDACs to an individual affected with a TBI, wherein said effective dose is an amount of AMDACs sufficient, e.g., to cause a detectable improvement in, reduce the severity of, or reduce the progression of one or more symptoms of mild TBI, including, but not limited to, cognitive problems such as headache, memory problems, attention deficits, mood swings and frustration, fatigue, visual disturbances, memory loss, poor attention/concentration, sleep disturbances, dizziness/loss of balance, irritability, emotional disturbances, feelings of depression, seizures, nausea, loss of smell, sensitivity to light and sounds, mood changes, getting lost or confused, or slowness in thinking.

In specific embodiments, the effective dose is an amount of AMDACs sufficient to treat a concussion, e.g., to cause a detectable improvement in, reduce the severity of, or reduce the progression of, one or more symptoms of a concussion, including, but not limited to, confusion or feeling dazed, clumsiness, slurred speech, nausea or vomiting, headache, balance problems or dizziness, blurred vision, sensitivity to light, sensitivity to noise, sluggishness, ringing in ears, behavior or personality changes, concentration difficulties, or memory loss. In some embodiments, the concussion is a Grade 1 (mild) concussion, characterized by no loss of consciousness and concussion symptoms lasting for less than minutes. In some embodiments, the concussion is a Grade 2 (moderate) concussion, characterized by no loss of consciousness and concussion symptoms lasting for longer than 15 minutes. In some embodiments, the concussion is a Grade 3 (severe) concussion, characterized by a loss of consciousness of at least a few seconds.

In some embodiments, the invention provides for the administration of an effective dose of AMDACs to an individual affected with a TBI, wherein said effective dose is an amount of AMDACs sufficient, e.g., to cause a detectable improvement in, reduce the severity of, or reduce the progression of, one or more symptoms of moderate to severe TBI, including, but not limited to, cognitive deficits such as difficulties with attention, concentration, distractibility, memory, speed of processing, confusion, perseveration, impulsiveness, language processing, speech and language, not understanding the spoken word (receptive aphasia), difficulty speaking and being understood (expressive aphasia), slurred speech, speaking very fast or very slow, problems reading, problems writing; sensory deficits, such as difficulties with interpretation of touch, temperature, movement, limb position or fine discrimination; perceptual deficits, such as difficulty with the integration or patterning of sensory impressions into psychologically meaningful data; visual deficits, including partial or total loss of vision, weakness of eye muscles and double vision (diplopia), blurred vision, problems judging distance, involuntary eye movements (nystagmus), intolerance of light (photophobia); hearing deficits, including a decrease or loss of hearing, or ringing in the ears (tinnitus), or increased sensitivity to sounds; olfactory deficits, including loss or diminished sense of smell (anosmia); loss or diminished sense of taste; seizures, including the convulsions associated with epilepsy that can be several types and can involve disruption in consciousness, sensory perception, or motor movement; physical changes, including physical paralysis/spasticity; chronic pain, loss of control of bowel and bladder, sleep disorders, loss of stamina, appetite changes, dysregulation of body temperature, and menstrual difficulties; social-emotional difficulties, including dependent behaviors, lack of emotional ability, lack of motivation, irritability, aggression, depression, disinhibition, or denial/lack of awareness.

In one embodiment, the invention provides for the administration of an effective dose of AMDACs to an individual affected with a TBI, wherein said effective dose is an amount of AMDACs sufficient, e.g., to cause a detectable improvement in, reduce the severity of, or reduce the progression of, one or more symptoms of TBI listed above. In certain embodiments, the particular technique(s) for detecting an improvement in, a reduction in the severity of, or a reduction in the progression of, one or more symptoms, conditions, or syndromes of TBI is not critical to the method of treating TBI provided herein. In certain embodiments, the assessment of said improvement or reduction in the progression of one or more symptoms of SCI is determined according to the judgment of a practitioner in the art. In certain embodiments, the assessment of said improvement or reduction in the progression of one or more symptoms of TBI is determined according to the judgment of a practitioner in the art in combination with the subjective experience of the subject.

In some embodiments, an improvement in one or more symptoms of, or a reduction in the progression of one or more symptoms of, said TBI is detected in accordance with the Glasgow Coma Scale (GCS). The GCS defines the severity of a TBI within 48 hours of injury as follows:

Eye Opening

Spontaneous=4

To speech=3

To painful stimulation=2

No response=1

Motor Response

Follows commands=6

Makes localizing movements to pain=5

Makes withdrawal movements to pain=4

Flexor (decorticate) posturing to pain=3

Extensor (decerebrate) posturing to pain=2

No response=1

Verbal Response

Oriented to person, place, and date=5

Converses but is disoriented=4

Says inappropriate words=3

Says incomprehensible sounds=2

No response=1
The severity of TBI according to the GCS score (within 48 h) is as follows: Vegetative TBI=less than 3 (characterized by sleep wake cycles; arousal, but no interaction with environment; no localized response to pain)
Severe TBI=3-8 (characterized by coma: unconscious state; no meaningful response, no voluntary activities)
Moderate TBI=9-12 (characterized by loss of consciousness greater than 30 minutes; physical or cognitive impairments which may or may resolve; patient may benefit from rehabilitation)
Mild TBI=13-15 (characterized by a brief change in mental status (confusion, disorientation or loss of memory) or loss of consciousness for less than 30 minutes)

Thus, in a specific embodiment of the method of treating TBI provided herein, the therapeutically effective amount of AMDACs is an amount sufficient to cause a 1, 2, 3, 4, 5, 6, 7, 8, 9, 10, 11, 12 or higher, point increase in the GCS score of the patient. In some embodiments, the therapeutically effective amount of AMDACs is an amount sufficient to cause a 1, 2, or 3 point increase with regard to eye opening, in accordance with the GCS. In some embodiments, the therapeutically effective amount of AMDACs is an amount sufficient to cause a 1, 2, 3, 4 or 5 point increase with regard to motor response, in accordance with the GCS. In some embodiments, the therapeutically effective amount of AMDACs is an amount sufficient to cause a 1, 2, 3 or 4 point increase with regard to verbal response, in accordance with the GCS. In some embodiments, the therapeutically effective amount of AMDACs is an amount sufficient to reduce the severity of the traumatic injury from a level corresponding to vegetative TBI to a level corresponding to severe, moderate or mild TBI. In some embodiments, the therapeutically effective amount of AMDACs is an amount sufficient to reduce the severity of the traumatic injury from a level corresponding to severe TBI to a level corresponding to moderate or mild TBI. In some embodiments, the therapeutically effective amount of AMDACs is an amount sufficient to reduce the severity of the traumatic injury from a level corresponding to moderate TBI to a level corresponding to mild TBI.

In some embodiments, an improvement in one or more symptoms of, or a reduction in the progression of one or more symptoms of, said TBI is detected in accordance with the Ranchos Los Amigos scale. The Ranchos Los Amigos Scale measures the levels of awareness, cognition, behavior and interaction with the environment, according to the following scale:

Level I: No Response

Level II: Generalized Response

Level III: Localized Response

Level IV: Confused-agitated

Level V: Confused-inappropriate

Level VI: Confused-appropriate

Level VII: Automatic-appropriate

Level VIII: Purposeful-appropriate

Thus, in a specific embodiment of the method of treating TBI provided herein, the therapeutically effective amount of AMDACs is an amount sufficient to cause a one, two, three, four, five, six or seven level increase in the score of the patient according to the Rancho Los Amigos Scale. In some embodiments, the therapeutically effective amount of AMDACs is an amount sufficient to raise the subject's awareness, cognition, behavior and interaction with the environment from a level of no response to a level of generalized response, localized response, confused agitation, confused inappropriate response, confused appropriate response, automatic appropriate response or purposeful appropriate response. In some embodiments, the therapeutically effective amount of AMDACs is an amount sufficient to raise the subject's awareness, cognition, behavior and interaction with the environment from a level of generalized response to a level of localized response, confused agitation, confused inappropriate response, confused appropriate response, automatic appropriate response or purposeful appropriate response. In some embodiments, the therapeutically effective amount of AMDACs is an amount sufficient to raise the subject's awareness, cognition, behavior and interaction with the environment from a level of localized response to a level of confused agitation, confused inappropriate response, confused appropriate response, automatic appropriate response or purposeful appropriate response. In some embodiments, the therapeutically effective amount of AMDACs is an amount sufficient to raise the subject's awareness, cognition, behavior and interaction with the environment from a level of confused agitation to a level of confused inappropriate response, confused appropriate response, automatic appropriate response or purposeful appropriate response. In some embodiments, the therapeutically effective amount of AMDACs is an amount sufficient to raise the subject's awareness, cognition, behavior and interaction with the environment from a level of confused inappropriate response to a level of confused appropriate response, automatic appropriate response or purposeful appropriate response. In some embodiments, the therapeutically effective amount of AMDACs is an amount sufficient to raise the subject's awareness, cognition, behavior and interaction with the environment from a level of confused appropriate response to a level of automatic appropriate response or purposeful appropriate response. In some embodiments, the therapeutically effective amount of AMDACs is an amount sufficient to raise the subject's awareness, cognition, behavior and interaction with the environment from a level of automatic appropriate response to a level of purposeful appropriate response.

An individual having, or experiencing, a symptom of, TBI, can be treated with a plurality of AMDACs, and, optionally, one or more therapeutic agents, at any time during the progression of the injury. For example, the individual can be treated immediately after injury, or within 1, 2, 3, 4, 5, 6 days of injury, or within 1, 2, 3, 4, 5, 6, 7, 8, 9, 10, 15, 20, 25, 30, 35, 40, 45, 50 days or more of injury, or within 1, 2, 3, 4, 5, 6, 7, 8, 9, 10 or more years after injury. The individual can be treated once, or multiple times during the clinical course of the injury. In a specific embodiment of the method of treatment, said AMDACs are administered to said individual within 21 days of development of one or more symptoms of a traumatic brain injury. In another specific embodiment of the method of treatment, said AMDACs are administered to said individual within 14 days of development of one or more symptoms of a traumatic brain injury. In another specific embodiment of the method of treatment, said AMDACs are administered to said individual within 7 days of development of one or more symptoms of a traumatic brain injury. In another specific embodiment of the method of treatment, said AMDACs are administered to said individual within 48 hours of development of one or more symptoms of a traumatic brain injury. In another specific embodiment, said AMDACs are administered to said individual within 24 hours of development of one or more symptoms of a traumatic brain injury. In another specific embodiment, said AMDACs are administered to said individual within 12 hours of development of one or more symptoms of a traumatic brain injury. In another specific embodiment, said AMDACs are administered to said individual within 3 hours of development of one or more symptoms of a traumatic brain injury.

In certain embodiments of the invention, the individual is an animal, preferably a mammal, more preferably a non-human primate. In certain embodiments, the individual is a human patient. The individual can be a male or female subject. In certain embodiments, the subject is a non-human animal, such as, for instance, a cow, sheep, goat, horse, dog, cat, rabbit, rat or mouse.

The AMDACs useful in the treatment of TBI can be any of the AMDACs disclosed herein. In a specific embodiment, the AMDACs are OCT-4⁻ (negative for OCT-4, also known as POU5F1 or octamer binding protein 4). In another specific embodiment, the AMDACs are OCT-4⁻ and VEGFR1/Flt-1⁺ (vascular endothelial growth factor receptor 1) and/or VEGFR2/KDR⁺ (vascular endothelial growth factor receptor 2, also known as kinase insert domain receptor). In another specific embodiment, the AMDACs are OCT-4⁻ and CD49f⁺ (integrin-α6⁺). In another specific embodiment, the AMDACs are OCT-4⁻ and HLA-G⁻. In another specific embodiment, the AMDACs are OCT-4⁻ and CD90⁺, CD105⁺, or CD117⁻. In another specific embodiment, the AMDACs are OCT-4⁻, CD90⁺, CD105⁺, and CD117⁻. In another specific embodiment, the AMDACs are OCT-4⁻ and do not express SOX2. In another specific embodiment, the AMDACs are GFAP⁺. In another specific embodiment, the AMDACs are beta-tubulin III (Tuj1)⁺. In another specific embodiment, the AMDACs are OCT-4⁻, GFAP⁺, and beta-tubulin III (Tuj1)⁺. In another specific embodiment, the AMDACs useful in the treatment of TBI are OCT-4⁻, CD200⁺, CD105⁺, and CD49f⁺. In another specific embodiment, the AMDACs useful in the treatment of TBI are CD200⁺, CD105⁺, CD90⁺, and CD73⁺. In another specific embodiment, the AMDACs useful in the treatment of TBI are CD117⁻ and not selected using an antibody to CD117. In another specific embodiment, the AMDACs useful in the treatment of TBI are CD146⁻ and not selected using an antibody to CD146. In another specific embodiment, the AMDACs useful in the treatment of TBI are OCT-4⁻ and do not express CD34 following induction with VEGF. In another specific embodiment, the AMDACs useful in the treatment of TBI are neurogenic, as determinable by a short-term neural differentiation assay (see, e.g., Section 5.12.1, below). In another specific embodiment, the AMDACs useful in the treatment of TBI are non-chondrogenic as determinable by an in vitro chondrogenic potential assay (see, e.g., Section 5.12.3, below). In another specific embodiment, the AMDACs useful in the treatment of TBI are non-osteogenic as determinable by an osteogenic phenotype assay (see, e.g., Section 5.12.2, below).

In a specific embodiment, the AMDACs useful in the treatment of TBI are telomerase⁻, as measured by RT-PCR and/or TRAP assays. In another specific embodiment, the AMDACs useful in the treatment of TBI do not express mRNA for telomerase reverse transcriptase (TERT) as determinable by RT-PCR, e.g., for 30 cycles. In another specific embodiment, the AMDACs useful in the treatment of TBI are NANOG⁻, as measured by RT-PCR. In another specific embodiment, the AMDACs useful in the treatment of TBI do not express mRNA for NANOG as determinable by RT-PCR, e.g., for 30 cycles. In a specific embodiment, the AMDACs useful in the treatment of TBI are (sex determining region Y)-box 2 (SOX2)⁻. In another specific embodiment, the AMDACs useful in the treatment of TBI do not express mRNA for SOX2 as determinable by RT-PCR, e.g., for 30 cycles. In another specific embodiment, the AMDACs useful in the treatment of TBI are not osteogenic as measured by an osteogenic phenotype assay (see, e.g., Section 5.12.2, below). In another specific embodiment, the AMDACs useful in the treatment of TBI are not chondrogenic as measured by a chondrogenic potential assay (see, e.g., Section 5.12.3, below). In another specific embodiment, the AMDACs useful in the treatment of TBI are not osteogenic as measured by an osteogenic phenotype assay (see, e.g., Section 5.12.2, below) and are not chondrogenic as measured by a chondrogenic potential assay (see, e.g., Section 5.12.3, below).

In one embodiment, the individual is administered a dose of about 300 million AMDACs. Dosage, however, can vary according to the individual's physical characteristics, e.g., weight, and can range from 1 million to 10 billion AMDACs per dose, preferably between 10 million and 1 billion per dose, or between 100 million and 500 million AMDACs per dose. The administration is preferably intravenous, but can be by any medically-acceptable route for the administration of live cells, e.g., intravenous, intraarterial, intraperitoneal, intraventricular, intrasternal, intracranial, intramuscular, intrasynovial, intraocular, intravitreal (e.g., where there is an ocular involvement), intracerebral, intracerebroventricular (e.g., where there is a neurologic or brain involvement), intrathecal, intraosseous infusion, intravesical, transdermal, intracisternal, epidural, or subcutaneous administration. In specific embodiments, administration is by bolus injection or infusion directly into the site of the traumatic brain injury, e.g., via cisterna magna.

AMDACs, or medium conditioned by AMDACs, can be administered in a single dose, or in multiple doses. Where AMDACs are administered in multiple doses, the doses can be part of a therapeutic regimen designed to relieve one or more acute symptoms of TBI, or can be part of a long-term therapeutic regimen designed to lessen the severity of TBI.

The methods for treating TBI provided herein further encompass treating TBI by administering a therapeutically effective amount of AMDACs in conjunction with one or more therapies or treatments used in the course of treating TBI. The one or more additional therapies may be used prior to, concurrent with, or after administration of the AMDACs. In some embodiments, the one or more additional therapies comprise surgical treatment. In some embodiments, a bolt or ICP (intracranial pressure) monitoring device may be placed in the skull to monitor pressure in the brain cavity. In some embodiments, where there is bleeding in the skull cavity, this may be surgically removed or drained, and bleeding vessels or tissue may be surgically repaired prior to, concurrent with, or after administration of the AMDACs. In severe cases, if there is extensive swelling and damaged brain tissue, a portion may be surgically removed, to make room for the living brain tissue, prior to, concurrent with, or after administration of the AMDACs. In some embodiments, the one or more additional therapies comprise the use of mechanical ventilation, which supports breathing and helps keep the pressure down in the head.

Also provided herein are methods for the treatment of an individual having, or experiencing, a symptom of, TBI, comprising administering to the individual a plurality of AMDACs sufficient to cause a detectable improvement in one or more symptoms, or a reduction in the progression of one or more symptoms of, said traumatic brain injury, and one or more therapeutic agents. For example, AMDACs can be administered in conjunction with medications to sedate and put the subject in a drug-induced coma to minimize agitation and secondary injury. In some embodiments, seizure prevention medications may be given early in the course of treatment and later if the individual has seizures. In some embodiments, medications to control spasticity may be used as the patient recovers function. In addition, medications may be used to improve attention and concentration (e.g., amantadine and methylphenidate, bromocriptine and antidepressants), or to control aggressive behavior (e.g., carbamamazapine and amitriptyline).

In a specific embodiment, the TBI treated in accordance with the methods described herein results from or is caused by a non-ischemic event. In another specific embodiment, the TBI treated in accordance with the methods described herein is not a hematoma or does not result from a hematoma. In another specific embodiment, the TBI treated in accordance with the methods described herein is not a hematoma that caused by external force on the skull. In another specific embodiment, the TBI treated in accordance with the methods described herein is not caused by a disruption of the flow of blood in or around the brain of the individual suffering from the TBI.

5.2 Use of Amnion Derived Adherent Cells to Suppress an Inflammatory Response Caused by or Associated with a CNS Injury

In another aspect, provided herein is a method of treating an individual having a CNS injury comprising suppressing an inflammatory response caused by or associated with the CNS injury. Provided herein are methods for the modulation, e.g., suppression, of the activity, e.g., proliferation, of an immune cell, or plurality of immune cells, by contacting the immune cell(s) with a plurality of the amnion derived adherent cells (AMDACs) described herein. Amnion derived adherent cell-mediated immunomodulation, e.g., immunosuppression, would, for example, be advantageous for a CNS injury wherein inflammation plays a role in either or both the early and chronic stages of the CNS injury.

In one embodiment, provided herein is a method of suppressing an immune response in an individual caused by or associated with a CNS injury, e.g., a spinal cord injury or traumatic brain injury, to the individual, comprising contacting a plurality of the individual's immune cells with a plurality of amnion derived adherent cells for a time sufficient for said amnion derived adherent cells to detectably suppress the immune response, wherein said amnion derived adherent cells detectably suppress T cell proliferation in, e.g., a mixed lymphocyte reaction (MLR) assay or a regression assay.

Amnion derived adherent cells are, e.g., the amnion derived adherent cells described elsewhere herein (see Section 5.4). Amnion derived adherent cells used for immunosuppression can be derived or obtained from the amnion of a single placenta or the amnions from multiple placentas. Amnion derived adherent cells used for immunosuppression can also be derived from a single species, e.g., the species of the intended recipient or the species of the immune cells the function of which is to be reduced or suppressed, or can be derived from multiple species.

An “immune cell” in the context of this method, and the methods disclosed herein, means any cell of the immune system, particularly T cells and natural killer (NK) cells. Thus, in various embodiments of the method, amnion derived adherent cells are contacted with a plurality of immune cells, wherein the plurality of immune cells are, or comprises, a plurality of T cells (e.g., a plurality of CD3⁺ T cells, CD4⁺ T cells and/or CD8⁺ T cells) and/or natural killer cells. An “immune response” in the context of the method can be any response by an immune cell to a stimulus normally perceived by an immune cell, e.g., a response to the presence of an antigen. In various embodiments, an immune response can be the proliferation of T cells (e.g., CD3⁺ T cells, CD4⁺ T cells and/or CD8⁺ T cells) in response to a CNS injury, e.g., a spinal cord injury or traumatic brain injury. The immune response can also be any activity of a NK cell, the maturation of a dendritic cell, or the like. The immune response can also be a local, tissue- or organ-specific, or systemic effect of an activity of one or more classes of immune cells, e.g., the immune response can be inflammation, formation of inflammation-related scar tissue, and the like.

“Contacting” in this context encompasses bringing the amnion derived adherent cells and immune cells together in a single container (e.g., culture dish, flask, vial, etc.) or in vivo, for example, in the same individual (e.g., mammal, for example, human). In a preferred embodiment, the contacting is for a time sufficient, and with a sufficient number of amnion derived adherent cells and immune cells, that a change in an immune function of the immune cells is detectable. More preferably, in various embodiments, said contacting is sufficient to suppress immune function (e.g., T cell proliferation in response to an antigen) by at least 50%, 60%, 70%, 80%, 90% or 95%, compared to the immune function in the absence of the amnion derived adherent cells. Such suppression in an in vivo context can be determined in an in vitro assay (see below); that is, the degree of suppression in the in vitro assay can be extrapolated, for a particular number of amnion derived adherent cells and a number of immune cells in a recipient individual, to a degree of suppression in the individual.

In certain embodiments, provided herein are methods of using amnion derived adherent cells to modulate an immune response, or the activity of a plurality of one or more types of immune cells, in vitro. Contacting the amnion derived adherent cells and plurality of immune cells can comprise combining the amnion derived adherent cells and immune cells in the same physical space such that at least a portion of the plurality of amnion derived adherent cells interacts with at least a portion of the plurality of immune cells; maintaining the amnion derived adherent cells and immune cells in separate physical spaces with common medium; or can comprise contacting medium from one or a culture of amnion derived adherent cells or immune cells with the other type of cell (for example, obtaining culture medium from a culture of amnion derived adherent cells and resuspending isolated immune cells in the medium). In a specific example, the contacting is performed in a Mixed Lymphocyte Reaction (MLR). In another specific example, the contacting is performed in a regression assay or beat T cell reaction (BTR) assay.

Such contacting can, for example, take place in an experimental setting designed to determine the extent to which a particular plurality of amnion derived adherent cells is immunomodulatory, e.g., immunosuppressive. Such an experimental setting can be, for example, a mixed lymphocyte reaction (MLR) or regression assay. Procedures for performing the MLR and regression assays are well-known in the art. See, e.g. Schwarz, “The Mixed Lymphocyte Reaction: An In Vitro Test for Tolerance,” J. Exp. Med. 127(5):879-890 (1968); Lacerda et al., “Human Epstein-Barr Virus (EBV)-Specific Cytotoxic T Lymphocytes Home Preferentially to and Induce Selective Regressions of Autologous EBV-Induced B Lymphoproliferations in Xenografted C.B-17 Scid/Scid Mice,” J. Exp. Med. 183:1215-1228 (1996). In a preferred embodiment, an MLR is performed in which pluralities of amnion derived adherent cells are contacted with a plurality of immune cells (e.g., lymphocytes, for example, CD3⁺, CD4⁺ and/or CD8⁺ T lymphocytes).

The MLR can be used to determine the immunosuppressive capacity of a plurality of amnion derived adherent cells. For example, a plurality of amnion derived adherent cells can be tested in an MLR comprising combining CD4⁺ or CD8⁺ T cells, dendritic cells (DC) and amnion derived adherent cells in a ratio of about 10:1:2, wherein the T cells are stained with a dye such as, e.g., CFSE that partitions into daughter cells, and wherein the T cells are allowed to proliferate for about 6 days. The plurality of amnion derived adherent cells is immunosuppressive if the T cell proliferation at 6 days in the presence of amnion derived adherent cells is detectably reduced compared to T cell proliferation in the presence of DC and absence of amnion derived adherent cells. In such an MLR, amnion derived adherent cells are either thawed or harvested from culture. About 20,000 amnion derived adherent cells are resuspended in 100 μl of medium (RPMI 1640, 1 mM HEPES buffer, antibiotics, and 5% pooled human serum), and allowed to attach to the bottom of a well for 2 hours. CD4⁺ and/or CD8⁺ T cells are isolated from whole peripheral blood mononuclear cells Miltenyi magnetic beads. The cells are CFSE stained, and a total of 100,000 T cells (CD4⁺ T cells alone, CD8⁺ T cells alone, or equal amounts of CD4⁺ and CD8⁺ T cells) are added per well. The volume in the well is brought to 200 μl, and the MLR is allowed to proceed.

In one embodiment, therefore, provided herein is a method of suppressing an immune response comprising contacting a plurality of immune cells with a plurality of amnion derived adherent cells for a time sufficient for said amnion derived adherent cells to detectably suppress T cell proliferation in a mixed lymphocyte reaction (MLR) assay or in a regression assay. In one embodiment, said amnion derived adherent cells used in the MLR represent a sample or aliquot of amnion derived adherent cells from a larger population of amnion derived adherent cells.

Populations of amnion derived adherent cells obtained from different placentas, or different tissues within the same placenta, can differ in their ability to modulate an activity of an immune cell, e.g., can differ in their ability to suppress T cell activity or proliferation or NK cell activity. It is thus desirable to determine, prior to use, the capacity of a particular population of amnion derived adherent cells for immunosuppression. Such a capacity can be determined, for example, by testing a sample of the amnion derived adherent cell population in an MLR or regression assay. In one embodiment, an MLR is performed with the sample, and a degree of immunosuppression in the assay attributable to the amnion derived adherent cells is determined. This degree of immunosuppression can then be attributed to the amnion derived adherent cell population that was sampled. Thus, the MLR can be used as a method of determining the absolute and relative ability of a particular population of amnion derived adherent cells to suppress immune function. The parameters of the MLR can be varied to provide more data or to best determine the capacity of a sample of amnion derived adherent cells to immunosuppress. For example, because immunosuppression by amnion derived adherent cells appears to increase roughly in proportion to the number of amnion derived adherent cells present in the assay, the MLR can be performed with, in one embodiment, two or more numbers of amnion derived adherent cells, e.g., 1×10³, 3×10³, 1×10⁴ and/or 3×10⁴ amnion derived adherent cells per reaction. The number of amnion derived adherent cells relative to the number of T cells in the assay can also be varied. For example, amnion derived adherent cells and T cells in the assay can be present in any ratio of, e.g. about 100:1 to about 1:100, preferably about 1:5, though a relatively greater number of amnion derived adherent cells or T cells can be used.

The regression assay or BTR assay can be used in similar fashion.

Therefore, provided herein are methods of using amnion derived adherent cells to modulate an immune response, or the activity of a plurality of one or more types of immune cells, in vivo, for example, an immune response caused by or associated with a CNS injury, e.g., a spinal cord injury or traumatic brain injury. Amnion derived adherent cells and immune cells can be contacted, e.g., in an individual that is a recipient of a plurality of amnion derived adherent cells. Where the contacting is performed in an individual, in one embodiment, the contacting is between exogenous amnion derived adherent cells (that is, amnion derived adherent cells not derived from the individual or an amnion associated with the individual) and a plurality of immune cells endogenous to the individual. In specific embodiments, the immune cells within the individual are CD3⁺ T cells, CD4⁺ T cells, CD8⁺ T cells, and/or NK cells.

The amnion derived adherent cells can be administered to the individual in a ratio, with respect to the known or expected number of immune cells, e.g., T cells, in the individual, of from about 10:1 to about 1:10, preferably about 1:5. However, a plurality of amnion derived adherent cells can be administered to an individual in a ratio of, in non-limiting examples, about 10,000:1, about 1,000:1, about 100:1, about 10:1, about 1:1, about 1:10, about 1:100, about 1:1,000 or about 1:10,000. Generally, about 1×10⁵ to about 1×10⁸ amnion derived adherent cells per recipient kilogram, preferably about 1×10⁶ to about 1×10⁷ amnion derived adherent cells per recipient kilogram can be administered to effect immunosuppression. In various embodiments, a plurality of amnion derived adherent cells administered to an individual or subject comprises at least, about, or no more than, 1×10⁵, 3×10⁵, 1×10⁶, 3×10⁶, 1×10⁷, 3×10⁷, 1×10⁸, 3×10⁸, 1×10⁹, 3×10⁹ amnion derived adherent cells, or more.

The amnion derived adherent cells can also be administered with one or more second types of stem cells, e.g., mesenchymal stem cells from bone marrow. Such second stem cells can be administered to an individual with amnion derived adherent cells in a ratio of, e.g., about 1:10 to about 10:1.

To facilitate contacting, or proximity of, the amnion derived adherent cells and immune cells in vivo, the amnion derived adherent cells can be administered to the individual by any route sufficient to bring the amnion derived adherent cells and immune cells into contact with each other. For example, the amnion derived adherent cells can be administered to the individual, e.g., intravenously, intramuscularly, intraperitoneally, intraocularly, parenterally, intrathecally, or directly into an organ, e.g., pancreas. For in vivo administration, the amnion derived adherent cells can be formulated as a pharmaceutical composition, as described in Section 5.8.1, below.

The method of immunosuppression can additionally comprise the addition of one or more immunosuppressive agents, particularly in the in vivo context. In one embodiment, the plurality of amnion derived adherent cells are contacted with the plurality of immune cells in vivo in an individual, and a composition comprising an immunosuppressive agent is administered to the individual. Immunosuppressive agents are well-known in the art and include, e.g., anti-T cell receptor antibodies (monoclonal or polyclonal, or antibody fragments or derivatives thereof), anti-IL-2 receptor antibodies (e.g., Basiliximab (SIMULECT®) or daclizumab (ZENAPAX)®), anti T cell receptor antibodies (e.g., Muromonab-CD3), azathioprine, corticosteroids, cyclosporine, tacrolimus, mycophenolate mofetil, sirolimus, calcineurin inhibitors, and the like. In a specific embodiment, the immumosuppressive agent is a neutralizing antibody to macrophage inflammatory protein (MIP)-1α or MIP-1β. Preferably, the anti-MIP-1α or MIP-1β antibody is administered in an amount sufficient to cause a detectable reduction in the amount of MIP-1α and/or MIP-1β in said individual.

Amnion derived adherent cells, in addition to suppression of proliferation of T cells, have other anti-inflammatory effects on cells of the immune system which can be beneficial in the treatment of a CNS injury, e.g., a spinal cord injury or traumatic brain injury. For example, amnion derived adherent cells, e.g., in vitro or in vivo, as when administered to an individual, reduce an immune response mediated by a Th1 and/or a Th17 T cell subset. In another aspect, provided herein is a method of inhibiting a pro-inflammatory response, e.g., a Th1 response or a Th17 response, either in vivo or in vitro, comprising contacting T cells (e.g., CD4⁺ T lymphocytes or leukocytes) with amnion derived adherent cells, i.e., the amnion derived adherent cells described herein. In a specific embodiment, said contacting detectably reduces Th1 cell maturation. In a specific embodiment of the method, said contacting detectably reduces the production of one or more of lymphotoxin-1α (LT-1α), interleukin-1β (IL-1β), IL-12, IL-17, IL-21, IL-23, tumor necrosis factor alpha (TNFα) and/or interferon gamma (IFNγ) by said T cells or by an antigen-producing cell. In another specific embodiment of the method, said contacting potentiates or upregulates a regulatory T cell (Treg) phenotype, and/or reduces expression in a dendritic cell (DC) and/or macrophage of biomolecules that promote a Th1 and/or Th17 response (e.g., CD80, CD83, CD86, ICAM-1, HLA-II). In a specific embodiment, said T cells are also contacted with IL-10, e.g., exogenous IL-10 or IL-10 not produced by said T cells, e.g., recombinant IL-10.

In another embodiment, provided herein is a method of reducing the production of pro-inflammatory cytokines from macrophages, comprising contacting the macrophages with an effective amount of amnion derived adherent cells. In another embodiment, provided herein is a method of increasing a number of tolerogenic cells, promoting tolerogenic functions of immune cells, and/or upregulating tolerogenic cytokines, e.g., from macrophages, comprising contacting immune system cells with an effective amount of amnion derived adherent cells. In a specific embodiment, said contacting causes activated macrophages to produce detectably more IL-10 than activated macrophages not contacted with said amnion derived adherent cells. In another embodiment, provided herein is a method of upregulating, or increasing the number of, anti-inflammatory T cells, comprising contacting immune system cells with an effective amount of amnion derived adherent cells.

In one embodiment, provided herein is a method of inhibiting a Th1 response in an individual having, or experiencing, a symptom of, a CNS injury, e.g., a spinal cord injury or traumatic brain injury, comprising administering to the individual an effective amount of amnion derived adherent cells, wherein said effective amount is an amount that results in a detectable decrease in a Th1 response in the individual. In another embodiment, provided herein is a method of inhibiting a Th17 response in an individual having, or experiencing, a symptom of, a CNS injury, e.g., a spinal cord injury or traumatic brain injury, comprising administering to the individual an effective amount of amnion derived adherent cells, wherein said effective amount is an amount that results in a detectable decrease in a Th17 response in the individual. In specific embodiments of these methods, said administering detectably reduces the production, by T cells or antigen presenting cells in said individual, of one or more of IL-1β, IL-12, IL-17, IL-21, IL-23, TNFα and/or IFNγ. In another specific embodiment of the method, said contacting potentiates or upregulates a regulatory T cell (Treg) phenotype, or modulates production in a dendritic cell (DC) and/or macrophage in said individual of markers the promote a Th1 or Th17 response. In another specific embodiment, the method comprises additionally administering IL-10 to said individual.

In another aspect, provided herein are amnion derived adherent cells, as described herein, that have been genetically engineered to express one or more anti-inflammatory cytokines. In a specific embodiment, said anti-inflammatory cytokines comprise IL-10.

5.3 Amnion Derived Adherent Cells

Generally, amnion derived adherent cells superficially resemble fibroblasts or mesenchymal cells in appearance, having a generally fibroblastoid shape. Such cells adhere to a cell culture surface, e.g., to tissue culture plastic. In certain embodiments of any of the AMDACs disclosed herein, the cells are human cells.

AMDACs provided herein display cellular markers that distinguish them from other amnion-derived, or placenta-derived, cells. In certain embodiments of each of the embodiments of AMDACs described herein, the AMDACs are isolated.

In one embodiment, amnion derived adherent cells are OCT-4⁻ (octamer binding protein 4), as determinable by RT-PCR. In another specific embodiment, OCT-4⁻ amnion derived adherent cells are CD49f⁺, as determinable, e.g., by immunolocalization (e.g., flow cytometry). In another specific embodiment, said OCT-4⁻ cells are HLA-G⁻, as determinable by RT-PCR. In another specific embodiment, the OCT-4⁻ cells are VEGFR1/Flt-1⁺ (vascular endothelial growth factor receptor 1) and/or VEGFR2/KDR⁺ (vascular endothelial growth factor receptor 2), as determinable by immunolocalization, e.g., flow cytometry. In a specific embodiment, OCT-4⁻ amnion derived adherent cells express at least 2 log less PCR-amplified mRNA for OCT-4 at, e.g., 20 cycles, than an equivalent number of NTERA-2 cells and RNA amplification cycles. In another specific embodiment, said OCT-4⁻ cells are CD90⁺, CD105⁺, or CD117⁻ as determinable, e.g., by immunolocalization (e.g., flow cytometry). In a more specific embodiment, said OCT-4⁻ cells are CD90⁺, CD105⁺, and CD117⁻ as determinable, e.g., by immunolocalization (e.g., flow cytometry). In a more specific embodiment, the cells are OCT-4⁻ or HLA-G⁻, and is additionally CD49f⁺, CD90⁺, CD105⁺, and CD117⁻ as determinable, e.g., by immunolocalization (e.g., flow cytometry). In a more specific embodiment, the cells are OCT-4⁻, HLA-G⁻, CD49f⁺, CD90⁺, CD105⁺, and CD117⁻ as determinable, e.g., by immunolocalization (e.g., flow cytometry). In another specific embodiment, the OCT-4⁻ cells do not express SOX2, e.g., as determinable by RT-PCR for 30 cycles. In a specific embodiment, therefore, the amnion derived adherent cells are OCT-4⁻, CD49f⁺, CD90⁺, CD105⁺, and CD117⁻, as determinable by immunolocalization (e.g., flow cytometry), and SOX2⁻, as determinable by RT-PCR, e.g., for 30 cycles.

In a specific embodiment, the AMDACs described herein are GFAP⁺ as determinable by, e.g., a short-term neural differentiation assay (see, e.g., Section 5.12.1, below). In another specific embodiment, AMDACs are beta-tubulin III (Tuj1)⁺ as determinable by, e.g., a short-term neural differentiation assay (see, e.g., Section 5.12.1, below). In another specific embodiment, the AMDACs are OCT-4⁻, GFAP⁺, and beta-tubulin III (Tuj1)⁺. In another specific embodiment, the AMDACs are OCT-4⁻, CD200⁺, CD105⁺, and CD49f⁺. In another specific embodiment, the AMDACs are CD200⁺, CD105⁺, CD90⁺, and CD73⁺. In another specific embodiment, the AMDACs and/or AMDAC cell populations described herein are CD117⁻ and are not selected using an antibody to CD117. In another specific embodiment, the AMDACs and/or AMDAC cell populations described herein are CD146⁻ and are not selected using an antibody to CD146. In another specific embodiment, the AMDACs described herein are OCT-4⁻ as determinable by RT-PCR and/or immunolocalization (e.g., flow cytometry) and do not express CD34 following induction with VEGF as determinable by RT-PCR and/or immunolocalization (e.g., flow cytometry). In another specific embodiment, the AMDACs described herein are neurogenic, as determinable by a short-term neural differentiation assay (see, e.g., Section 5.12.1, below). In another specific embodiment, the AMDACs described herein are non-chondrogenic as determinable by an in vitro chondrogenic potential assay (see, e.g., Section 5.12.3, below). In another specific embodiment, the AMDACs described herein are non-osteogenic as determinable by an osteogenic phenotype assay (see, e.g., Section 5.12.2, below). In another specific embodiment, the AMDACs described herein are non-osteogenic after being cultured for up to 6 weeks (e.g., for 2 weeks, for 4 weeks, or for 6 weeks) in DMEM at pH 7.4 (High glucose) supplemented with 100 nM dexamethasone, 10 mM β-glycerol phosphate, 50 μM L-ascorbic acid-2-phosphate, wherein osteogenesis is assessed using von Kossa staining; alizarin red staining; or by detecting the presence of osteopontin, osteocalcin, osteonectin, and/or bone sialoprotein by, e.g., RT-PCR.

In another embodiment, said OCT-4⁻ cells are one or more of CD29⁺, CD73⁺, ABC-p⁺, and CD38⁻, e.g., as determinable by immunolocalization (e.g., flow cytometry).

In another specific embodiment, for example, OCT-4⁻ AMDACs can additionally be one or more of CD9⁺, CD10⁺, CD44⁺, CD54⁺, CD98⁺, TEM-7⁺ (tumor endothelial marker 7), CD31⁻, CD34⁻, CD45⁻, CD133⁻, CD143⁻ (angiotensin-I-converting enzyme, ACE), CD146⁻ (melanoma cell adhesion molecule), or CXCR4⁻ (chemokine (C—X—C motif) receptor 4), e.g., as determinable by immunolocalization (e.g., flow cytometry), or HLA-G⁻ as determinable by RT-PCR. In a more specific embodiment, said cells are CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻, e.g., as determinable by immunolocalization (e.g., flow cytometry), and HLA-G⁻ as determinable by RT-PCR. In another embodiment, the amnion derived adherent cells are one or more of CD31⁻, CD34⁻, CD45⁻, and/or CD133⁻ as determinable, e.g., by immunolocalization (e.g., flow cytometry). In a specific embodiment, the amnion derived adherent cells are OCT-4⁻, as determinable by RT-PCR; VEGFR1/Flt-1⁺ and/or VEGFR2/KDR⁺, as determinable by immunolocalization (e.g., flow cytometry); and one or more, or all, of CD31⁻, CD34⁻, CD45⁻, and/or CD133⁻ as determinable, e.g., by immunolocalization (e.g., flow cytometry).

In another specific embodiment, said AMDACs are additionally VE-cadherin⁻ as determinable by immunolocalization, e.g., flow cytometry. In another specific embodiment, said OCT-4⁻ cells are, either alone or in combination with other markers, additionally positive for CD105⁺ and CD200⁺ as determinable by immunolocalization, e.g., flow cytometry. In another specific embodiment, said cells do not express CD34 as detected by immunolocalization, e.g., flow cytometry, after exposure to 1 to 100 ng/mL VEGF for 4 to 21 days. In more specific embodiments, said cells do not express CD34 as detected by immunolocalization, e.g., flow cytometry, after exposure to 25 to 75 ng/mL VEGF for 4 to 21 days, or to 50 ng/mL VEGF for 4 to 21 days. In even more specific embodiments, said cells do not express CD34 as detected by immunolocalization, e.g., flow cytometry, after exposure to 1, 2.5, 5, 10, 25, 50, 75 or 100 ng/mL VEGF for 4 to 21 days. In yet more specific embodiments, said cells do not express CD34 as detected by immunolocalization, e.g., flow cytometry, after exposure to 1 to 100 ng/mL VEGF for 7 to 14, e.g., 7, days.

In specific embodiments, the amnion derived adherent cells are OCT-4⁻, as determinable by RT-PCR, and one or more of VE-cadherin⁻, VEGFR2/KDR⁺, CD9⁺, CD54⁺, CD105⁺, and/or CD200⁺ as determinable by immunolocalization, e.g., flow cytometry. In a specific embodiment, the amnion derived adherent cells are OCT-4⁻, as determinable by RT-PCR, and VE-cadherin, VEGFR2/KDR⁺, CD9⁺, CD54⁺, CD105⁺, and CD200⁺ as determinable by immunolocalization, e.g., flow cytometry. In another specific embodiment, said cells do not express CD34, as detected by immunolocalization (e.g., flow cytometry), e.g., after exposure to 1 to 100 ng/mL VEGF for 4 to 21 days.

In another embodiment, the amnion derived adherent cells are OCT-4⁻, CD49f⁺, HLA-G⁻, CD90⁺, CD105⁺, and CD117⁻. In a more specific embodiment, said cells are one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, or CXCR4⁻, as determinable by immunolocalization, e.g., flow cytometry. In a more specific embodiment, said cells CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry. In another specific embodiment, said cells are additionally VEGFR1/Flt-1⁺ and/or VEGFR2/KDR⁺, as determinable by immunolocalization, e.g., flow cytometry; and one or more of CD31⁻, CD34⁻, CD45⁻, CD133⁻, and/or Tie-2⁻ as determinable by immunolocalization, e.g., flow cytometry. In another specific embodiment, said cells are additionally VEGFR1/Flt-1⁺, VEGFR2/KDR⁺, CD31⁻, CD34⁻, CD45⁻, CD133⁻, and Tie-2⁻ as determinable by immunolocalization, e.g., flow cytometry.

In another embodiment, the OCT-4− amnion derived adherent cells are additionally one or more, or all, of CD9⁺, CD10⁺, CD44⁺, CD49f⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309⁺), as determinable by immunolocalization, e.g., flow cytometry; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, and/or VE-cadherin⁻, as determinable by immunolocalization, e.g., flow cytometry, or SOX2⁻, as determinable by RT-PCR.

In certain embodiments, the isolated tissue culture plastic-adherent amnion derived adherent cells are CD49f⁺. In a specific embodiment, said CD49f⁺ cells are additionally one or more, or all, of CD9⁺, CD10⁺, CD44⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309⁺), as determinable by immunolocalization, e.g., flow cytometry; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, OCT-4⁻ and/or VE-cadherin⁻, as determinable by immunolocalization, e.g., flow cytometry, or SOX2⁻, as determinable by RT-PCR.

In certain other embodiments, the isolated tissue culture plastic-adherent amnion derived adherent cells are HLA-G⁻, CD90⁺, and CD117⁻. In a specific embodiment, said HLA-G⁻, CD90⁺, and CD117⁻ cells are additionally one or more, or all, of CD9⁺, CD10⁺, CD44⁺, CD49f⁺, CD54⁺, CD98⁺, CD105⁺, CD200⁺, Tie-2⁺, TEM-7⁺, VEGFR1/Flt-1⁺, and/or VEGFR2/KDR⁺ (CD309⁺), as determinable by immunolocalization, e.g., flow cytometry; or additionally one or more, or all, of CD31⁻, CD34⁻, CD38⁻, CD45⁻, CD133⁻, CD143⁻, CD144⁻, CD146⁻, CD271⁻, CXCR4⁻, OCT-4⁻ and/or VE-cadherin⁻, as determinable by immunolocalization, e.g., flow cytometry, or SOX2⁻, as determinable by RT-PCR.

In another embodiment, the isolated amnion derived adherent cells do not constitutively express mRNA for angiopoietin 4 (ANGPT4), angiopoietin-like 3 (ANGPTL3), cadherin 5, type 2 (CDH5), bone gamma-carboxyglutamate (gla) protein (BGLAP), CD31, CD34, chemokine (C—X—C motif) ligand 10 (CXCL10), distal-less homeobox 5 (DLX5), fibrinogen α chain (FGA), fibroblast growth factor 4 (FGF4), FMS-like tyrosine kinase 3 (FLT3), HLA-G, interferon γ (IFNG), leukocyte cell derived chemotaxin 1 (LECT1), leptin (LEP), matrix metalloprotease 13 (MMP-13), NANOG, nestin, plasminogen (PLG), POU5F1 (OCT-4), prolactin (PRL), prokineticin 1 (PROK1), (sex determining region Y)-box 2 (SOX2), telomerase reverse transcriptase (TERT), tenomodulin (TNMD), and/or extracellular link domain containing 1 (XLKD1), as determinable by RT-PCR, e.g., for 30 cycles under standard culture conditions.

In other embodiments, isolated amnion derived adherent cells, or population of amnion derived adherent cells, express mRNA for (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB1R), and hepatoycte growth factor receptor (HGFR/c-met)

In another aspect, provided herein are isolated populations of cells, e.g., isolated populations of amnion cells or placental cells, or substantially isolated populations of AMDACs, comprising the amnion derived adherent cells described herein. The populations of cells can be homogeneous populations, e.g., a population of cells, at least about 90%, 95%, 98% or 99% of which are amnion derived adherent cells. The populations of cells can be heterogeneous, e.g., a population of cells wherein at most about 10%, 20%, 30%, 40%, 50%, 60%, 70% or 80% of the cells in the population are amnion derived adherent cells. The isolated populations of cells are not, however, tissue, i.e., amniotic membrane.

In one embodiment, provided herein is an isolated population of cells comprising AMDACs, e.g., a population of cells substantially homogeneous for AMDACs, or a population of cells heterogeneous with respect to the AMDACs, wherein said AMDACs are adherent to tissue culture plastic, and wherein said AMDACs are OCT-4⁻, as determinable by RT-PCR. In a specific embodiment, the AMDACs are CD49f⁺ or HLA-G⁻, e.g., as determinable by immunolocalization, e.g., flow cytometry, or RT-PCR. In another specific embodiment, said AMDACs in said population of cells are VEGFR1/Flt-1⁺ and/or VEGFR2/KDR⁺ as determinable by immunolocalization, e.g., flow cytometry, wherein said isolated population of cells is not an amnion or amniotic membrane or other tissue. In a more specific embodiment, the AMDACs in said population of cells are OCT-4⁻, and/or HLA-G⁻ as determinable by RT-PCR, and VEGFR1/Flt-1⁺ and/or VEGFR2/KDR⁺ as determinable by immunolocalization, e.g., flow cytometry. In another specific embodiment, said AMDACs are CD90⁺, CD105⁺, or CD117⁻. In a more specific embodiment, said AMDACs are CD90⁺, CD105⁺, and CD117⁻. In a more specific embodiment, the AMDACs are OCT-4⁻, CD49f⁺, CD90⁺, CD105⁺, and CD117⁻. In another specific embodiment, the AMDACs do not express SOX2, e.g., as determinable by RT-PCR for 30 cycles. In an even more specific embodiment, the population comprises AMDACs, wherein said AMDACs are OCT-4⁻, HLA-G⁻, CD49f⁺, CD90⁺, CD105⁺, and CD117⁻, as determinable by immunolocalization, e.g., flow cytometry, and SOX2⁻, e.g., as determinable by RT-PCR for 30 cycles.

In another specific embodiment, said AMDACs in said population of cells are CD90⁺, CD105⁺, or CD117⁻, as determinable by immunolocalization, e.g., flow cytometry. In a more specific embodiment, the AMDACs are CD90⁺, CD105⁺, and CD117⁻, as determinable by immunolocalization, e.g., flow cytometry. In a more specific embodiment, the AMDACs are OCT-4⁻ or HLA-G⁻, e.g., as determinable by RT-PCR, and are additionally CD49f⁺, CD90⁺, CD105⁺, and CD117⁻ as determinable by immunolocalization, e.g., flow cytometry. In a more specific embodiment, the AMDACs in said population of cells are OCT-4⁻, HLA-G⁻, CD49f⁺, CD90⁺, CD105⁺, and CD117⁻. In another specific embodiment, the AMDACs do not express SOX2, e.g., as determinable by RT-PCR for 30 cycles. In a more specific embodiment, therefore, the AMDACs are OCT-4⁻, CD49f⁺, CD90⁺, CD105⁺, and CD117⁻, as determinable by immunolocalization, e.g., flow cytometry, and SOX2⁻, as determinable by RT-PCR, e.g., for 30 cycles. In an even more specific embodiment, the AMDACs are OCT-4⁻ or HLA-G⁻, and are additionally CD49f⁺, CD90⁺, CD105⁺, and CD117⁻. In a more specific embodiment, the AMDACs are OCT-4⁻, HLA-G⁻, CD49f⁺, CD90⁺, CD105⁺, and CD117⁻.

In another embodiment, the amnion derived adherent cells in said population of cells are adherent to tissue culture plastic, OCT-4⁻ as determinable by RT-PCR, and VEGFR1/Flt-1⁺ and/or VEGFR2/KDR⁺ as determinable by immunolocalization, e.g., flow cytometry, and are additionally one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, or CXCR4⁻, as determinable by immunolocalization, e.g., flow cytometry, or HLA-G⁻ as determinable by RT-PCR, and wherein said isolated population of cells is not an amnion. In another embodiment, provided herein is an isolated population of cells comprising ? amnion derived adherent cells, wherein said cells are adherent to tissue culture plastic, wherein said cells are OCT-4⁻ as determinable by RT-PCR, and VEGFR1/Flt-1⁺ and/or VEGFR2/KDR⁺ as determinable by immunolocalization, e.g., flow cytometry, wherein said cells do not express CD34 as detected by immunolocalization, e.g., flow cytometry, after exposure to 1 to 100 ng/mL VEGF for 4 to 21 days, and wherein said isolated population of cells is not an amnion.

In a specific embodiment of any of the above embodiments, at least about 50%, 60%, 70%, 80%, 90%, 95%, 98% or 99% of cells in said population are said amnion derived adherent cells, as described or characterizable by any of the cellular marker combinations described above.

In another embodiment, any of the above populations of cells comprising amnion derived adherent cells forms sprouts or tube-like structures when cultured in the presence of an extracellular matrix protein, e.g., like collagen type I and IV, or an angiogenic factor, e.g., like vascular endothelial growth factor (VEGF), epithelial growth factor (EGF), platelet derived growth factor (PDGF) or basic fibroblast growth factor (bFGF), e.g., in or on a substrate such as placental collagen, e.g., or MATRIGEL™ for at least 4 days and up to 14 days.

In certain embodiments, provided herein is a cell that expresses, or a population of cells, wherein at least about 50%, 60%, 70%, 80%, 90%, 95% or 98% of cells in said isolated population of cells are amnion derived adherent cells that express RNA for one or more of, or all of, ACTA2 (actin, alpha 2, smooth muscle, aorta), ACTC1 (Actin, alpha cardiac muscle 1), ADAMTS1 (ADAM metallopeptidase with thrombospondin type 1 motif, 1), AMOT (angiomotin), ANG (angiogenin), ANGPT1 (angiopoietin 1), ANGPT2, ANGPTL1 (angiopoietin-like 1), ANGPTL2, ANGPTL4, BAH (brain-specific angiogenesis inhibitor 1), c-myc, CD44, CD140a, CD140b, CD200, CD202b, CD304, CD309, CEACAM1 (carcinoembryonic antigen-related cell adhesion molecule 1), CHGA (chromogranin A), COL15A1 (collagen, type XV, alpha 1), COL18A1 (collagen, type XVIII, alpha 1), COL4A1 (collagen, type IV, alpha 1), COL4A2 (collagen, type IV, alpha 2), COL4A3 (collagen, type IV, alpha 3), connexin-43, CSF3 (colony stimulating factor 3 (granulocyte), CTGF (connective tissue growth factor), CXCL12 (chemokine (CXC motif) ligand 12 (stromal cell-derived factor 1)), CXCL2, DNMT3B (DNA (cytosine-5-)-methyltransferase 3 beta), ECGF1 (thymidine phosphorylase), EDG1 (endothelial cell differentiation gene 1), EDIL3 (EGF-like repeats and discoidin I-like domains 3), ENPP2 (ectonucleotide pyrophosphatase/phosphodiesterase 2), EPHB2 (EPH receptor B2), FBLN5 (FIBULIN 5), F2 (coagulation factor II (thrombin)), FGF1 (acidic fibroblast growth factor), FGF2 (basic fibroblast growth factor), FIGF (c-fos induced growth factor (vascular endothelial growth factor D)), FLT4 (fms-related tyrosine kinase 4), FN1 (fibronectin 1), FST (follistatin), FOXC2 (forkhead box C2 (MFH-1, mesenchyme forkhead 1)), follistatin, Galectin-1, GRN (granulin), HGF (hepatocyte growth factor), HEY1 (hairy/enhancer-of-split related with YRPW motif 1), HSPG2 (heparan sulfate proteoglycan 2), IFNB1 (interferon, beta 1, fibroblast), IL8 (interleukin 8), IL12A, ITGA4 (integrin, alpha 4; CD49d), ITGAV (integrin, alpha V), ITGB3 (integrin, beta 3), KLF4 (Kruppel-like factor 4), MDK (midkine), MMP2 (matrix metalloprotease 2), MYOZ2 (myozenin 2), NRP2 (neuropilin 2), PDGFB (platelet-derived growth factor β), PF4 (platelet factor 4), PGK1 (phosphoglycerate kinase 1), PROX1 (prospero homeobox 1), PTN (pleiotrophin), SEMA3F (semophorin 3F), SERPINB5 (serpin peptidase inhibitor, Glade B (ovalbumin), member 5), SERPINC1, SERPINF1, TIMP2 (tissue inhibitor of metalloproteinases 2), TIMP3, TGFA (transforming growth factor, alpha), TGFB1, THBS1 (thrombospondin 1), THBS2, TIE1 (tyrosine kinase with immunoglobulin-like and EGF-like domains 1), TNF (tumor necrosis factor), TNNC1 (troponin C, type 1), TNNT2, TNFSF15 (tumor necrosis factor (ligand) superfamily, member 15), VASH1 (vasohibin 1), VEGF (vascular endothelial growth factor), VEGFB, VEGFC, and/or VEGFR1/FLT1 (vascular endothelial growth factor receptor 1).

When human cells are used, the gene designations throughout refer to human sequences, and, as is well known to persons of skill in the art, representative sequences can be found in literature, or in GenBank. Probes to the sequences can be determined by sequences that are publicly-available, or through commercial sources, e.g., specific TAQMAN® probes or TAQMAN® Angiogenesis Array (Applied Biosystems, part no. 4378710).

In certain embodiments, provided herein is a cell that expresses, or a population of cells, wherein at least about 50%, 60%, 70%, 80%, 90%, 95% or 98% of cells in said isolated population of cells are amnion derived adherent cells that express CD49d, Connexin-43, HLA-ABC, Beta 2-microglobulin, CD349, CD318, PDL1, CD106, Galectin-1, ADAM 17 precursor (A disintegrin and metalloproteinase domain 17) (TNF-alpha converting enzyme) (TNF-alpha convertase), Angiotensinogen precursor, Filamin A (Alpha-filamin) (Filamin 1) (Endothelial actin-binding protein) (ABP-280) (Nonmuscle filamin), Alpha-actinin 1 (Alpha-actinin cytoskeletal isoform) (Non-muscle alpha-actinin 1) (F-actin cross linking protein), Low-density lipoprotein receptor-related protein 2 precursor (Megalin) (Glycoprotein 330) (gp330), Macrophage scavenger receptor types I and II (Macrophage acetylated LDL receptor I and II), Activin receptor type JIB precursor (ACTR-IIB), Wnt-9 protein, Glial fibrillary acidic protein, astrocyte (GFAP), Myosin-binding protein C, cardiac-type (Cardiac MyBP-C) (C-protein, cardiac muscle isoform), Myosin heavy chain, nonmuscle type A (Cellular myosin heavy chain, type A) (Nonmuscle myosin heavy chain-A) (NMMHC-A), VEGF, HGF, IL-8, MCP-3, FGF2, Follistatin, G-CSF, EGF, ENA-78, GRO, IL-6, MCP-1, PDGF-BB, TIMP-2, uPAR, miR-17-3p, miR-18a, miR-18b, miR-19b, miR-92, miR-20a, miR-20b, members miRNA cluster 17-92, miR-296, miR-221, miR-222, miR-15b, and/or miR-16.

In one embodiment, provided herein are isolated amnion derived adherent cells, wherein said cells are adherent to tissue culture plastic, wherein said cells are OCT-4⁻, as determinable by RT-PCR, and CD49f⁺, HLA-G⁻, CD90⁺, CD105⁺, and CD117⁻, as determinable by immunolocalization, e.g., flow cytometry, and wherein said cells: (a) express one or more of CD9, CD10, CD44, CD54, CD98, CD200, Tie-2, TEM-7, VEGFR1/Flt-1, or VEGFR2/KDR (CD309), as determinable by immunolocalization, e.g., flow cytometry; (b) lack expression of CD31, CD34, CD38, CD45, CD133, CD143, CD144, CD146, CD271, CXCR4. HLA-G, or VE-cadherin, as determinable by immunolocalization, flow cytometry; (c) lack expression of SOX2, as determinable by RT-PCR; (d) express mRNA for ACTA2, ADAMTS1, AMOT, ANG, ANGPT1, ANGPT2, ANGPTL1, ANGPTL2, ANGPTL4, BAI1, c-myc, CD44, CD140a, CD140b, CD200, CD202b, CD304, CD309, CEACAM1, CHGA, COL15A1, COL18A1, COL4A1, COL4A2, COL4A3, Connexin-3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, Galectin-1, GRN, HGF, HEY1, HSPG2, IFNB1, IL8, IL12A, ITGA4, ITGAV, ITGB3, KLF-4, MDK, MMP2, MYOZ2, NRP2, PDGFB, PF4, PGK1, PROX1, PTN, SEMA3F, SERPINB5, SERPINC1, SERPINF1, TGFA, TGFB1, THBS1, THBS2, TIE1, TIMP2, TIMP3, TNF, TNNC1, TNNT2, TNFSF15, VASH1, VEGF, VEGFB, VEGFC, or VEGFR1/FLT1; (e) express one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; (f) secret VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the cell grows; (g) express micro RNAs miR-17-3p, miR-18a, miR-18b, miR-19b, miR-92, or miR-296 at a higher level than an equivalent number of bone marrow-derived mesenchymal stem cells; (h) express micro RNAs miR-20a, miR-20b, miR-221, miR-222, miR-15b, or miR-16 at a lower level than an equivalent number of bone marrow-derived mesenchymal stem cells; (i) express miRNAs miR-17-3p, miR-18a, miR-18b, miR-19b, miR-92, miR-20a, miR-20b, miR-296, miR-221, miR-222, miR-15b, or miR-16; and/or (j) express increased levels of CD202b, IL-8 or VEGF when cultured in less than about 5% O₂ compared to expression of CD202b, IL-8 or VEGF under 21% O₂. In a specific embodiment, the isolated amnion derived adherent cells are OCT-4⁻, as determinable by RT-PCR, and CD49f⁺, HLA-G⁻, CD90⁺, CD105⁺, and CD117⁻, as determinable by immunolocalization, e.g., flow cytometry, and (a) express CD9, CD10, CD44, CD54, CD90, CD98, CD200, Tie-2, TEM-7, VEGFR1/Flt-1, and VEGFR2/KDR (CD309), as determinable by immunolocalization, e.g., flow cytometry; (b) lack expression of CD31, CD34, CD38, CD45, CD133, CD143, CD144, CD146, CD271, CXCR4, HLA-G, and VE-cadherin, as determinable by immunolocalization, e.g., flow cytometry; (c) lack expression of SOX2, as determinable by RT-PCR; (d) express mRNA for ACTA2, ADAMTS1, AMOT, ANG, ANGPT1, ANGPT2, ANGPTL1, ANGPTL2, ANGPTL4, BAI1, c-myc, CD44, CD140a, CD140b, CD200, CD202b, CD304, CD309, CEACAM1, CHGA, COL15A1, COL18A1, COL4A1, COL4A2, COL4A3, Connexin-3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, Galectin-1, GRN, HGF, HEY1, HSPG2, IFNB1, IL8, IL12A, ITGA4, ITGAV, ITGB3, KLF-4, MDK, MMP2, MYOZ2, NRP2, PDGFB, PF4, PGK1, PROX1, PTN, SEMA3F, SERPINB5, SERPINC1, SERPINF1, TGFA, TGFB1, THBS1, THBS2, TIE1, TIMP2, TIMP3, TNF, TNNC1, TNNT2, TNFSF15, VASH1, VEGF, VEGFB, VEGFC, and/or VEGFR1/FLT1; (e) express one or more of CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A; (f) secrete VEGF, HGF, IL-8, MCP-3, FGF2, Follistatin, G-CSF, EGF, ENA-78, GRO, IL-6, MCP-1, PDGF-BB, TIMP-2, uPAR, and/or Galectin-1, e.g., into culture medium in which the cells grow; (g) express micro RNAs miR-17-3p, miR-18a, miR-18b, miR-19b, miR-92, and miR-296 at a higher level than an equivalent number of bone marrow-derived mesenchymal stem cells; (h) express micro RNAs miR-20a, miR-20b, miR-221, miR-222, miR-15b, and miR-16 at a lower level than an equivalent number of bone marrow-derived mesenchymal stem cells; (i) express miRNAs miR-17-3p, miR-18a, miR-18b, miR-19b, miR-92, miR-20a, miR-20b, miR-296, miR-221, miR-222, miR-15b, and miR-16; and/or (i) expresses increased levels of CD202b, IL-8 and VEGF when cultured in less than about 5% O₂ compared to expression of CD202b, IL-8 and/or VEGF when said cells are cultured under 21% O₂. Further provided herein are populations of cells comprising AMDACs, e.g. populations of AMDACs, having one or more of the above-recited characteristics.

In another embodiment, any of the above amnion derived adherent cells, or populations of cells comprising amnion derived adherent cells, take up acetylated low density lipoprotein (LDL) when cultured in the presence of extracellular matrix proteins, e.g., collagen type I or IV, and/or one or more angiogenic factors, e.g., VEGF, EGF, PDGF, or bFGF, e.g., on a substrate such as placental collagen or MATRIGEL™.

In another embodiment, the AMDACs are comprised within a population of cells. In specific embodiments of such embodiments, the amnion derived adherent cells are adherent to tissue culture plastic, are OCT-4⁻, as determinable by RT-PCR, and VEGFR2/KDR⁺, CD9⁺, CD54⁺, CD105⁺, CD200⁺, or VE-cadherin⁻, as determinable by immunolocalization, e.g., flow cytometry. In specific embodiments, at least 10%, 20%, 30%, 40%, 50%, 60%, 70%, 80%, 90%, 95%, 98% or 99% of the cells in said population of cells are amnion derived cells that are OCT-4⁻, as determinable by RT-PCR, and VEGFR2/KDR⁺, CD9⁺, CD54⁺, CD105⁺, CD200⁺, or VE-cadherin⁻, as determinable by immunolocalization, e.g., flow cytometry. In another specific embodiment, at least 10%, 20%, 30%, 40%, 50%, 60%, 70%, 80%, 90%, 95%, 98% or 99% of the cells in said population are amnion derived cells that are OCT-4⁻, as determinable by RT-PCR, and VEGFR2/KDR⁺, CD9⁺, CD54⁺, CD105⁺, CD200⁺, and VE-cadherin⁻, as determinable by immunolocalization, e.g., flow cytometry. In another specific embodiment, said cells that are OCT-4⁻, as determinable by RT-PCR, and VEGFR2/KDR⁺, CD9⁺, CD54⁺, CD105⁺, CD200⁺, or VE-cadherin⁻, as determinable by immunolocalization, e.g., flow cytometry, do not express CD34, as determinable by immunolocalization, e.g., flow cytometry, after exposure to 1 to 100 ng/mL VEGF for 4 to 21 days. In another specific embodiment, said cells are also VE-cadherin⁻.

In a specific embodiment, said amnion derived cells that are OCT-4⁻, as determinable by RT-PCR, and VEGFR2/KDR⁺, CD9⁺, CD54⁺, CD105⁺, CD200⁺, or VE-cadherin⁻, as determinable by immunolocalization, e.g., flow cytometry, form sprouts or tube-like structures when said population of cells is cultured in the presence of vascular endothelial growth factor (VEGF).

The amnion derived adherent cells described herein display the above characteristics, e.g., combinations of cell surface markers and/or gene expression profiles, in primary culture, or during proliferation in medium suitable for the culture of stem cells. Such medium includes, for example, medium comprising 1 to 100% DMEM-LG (Gibco), 1 to 100% MCDB-201 (Sigma), 1 to 10% fetal calf serum (FCS) (Hyclone Laboratories), 0.1 to 5× insulin-transferrin-selenium (ITS, Sigma), 0.1 to 5× linolenic-acid-bovine-serum-albumin (LA-BSA, Sigma), 10⁻⁵ to 10⁻¹⁵M dexamethasone (Sigma), 10⁻² to 10⁻¹⁰ M ascorbic acid 2-phosphate (Sigma), 1 to 50 ng/mL epidermal growth factor (EGF), (R&D Systems), 1 to 50 ng/mL platelet derived-growth factor (PDGF-BB) (R&D Systems), and 100 U penicillin/1000 U streptomycin. In a specific embodiment, the medium comprises 60% DMEM-LG (Gibco), 40% MCDB-201 (Sigma), 2% fetal calf serum (FCS) (Hyclone Laboratories), 1× insulin-transferrin-selenium (ITS), 1× linolenic-acid-bovine-serum-albumin (LA-BSA), 10⁻⁹ M dexamethasone (Sigma), 10⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin Other suitable media are described below.

The isolated populations of amnion derived adherent cells provided herein can comprise about, at least about, or no more than about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more amnion derived adherent cells, e.g., in a container. In various embodiments, at least 10%, 20%, 30%, 40%, 50%, 60%, 70%, 80%, 90%, 95%, or 99% of the cells in the isolated cell populations provided herein are amnion derived adherent cells. That is, a population of isolated amnion derived adherent cells can comprise, e.g., as much as 1%, 5%, 10%, 20%, 30%, 40%, 50%, 60%, 70%, 80%, 90% non-AMDAC cells. In other specific embodiments, at least 25%, 35%, 45%, 50%, 60%, 75%, 85% or more of the cells in the isolated population of cells comprising amnion derived adherent cells are not OCT-4⁺.

The amnion derived adherent cells provided herein can be cultured on a substrate. In various embodiments, the substrate can be any surface on which culture and/or selection of amnion derived adherent cells, can be accomplished. Typically, the substrate is plastic, e.g., tissue culture dish or multiwell plate plastic. Tissue culture plastic can be treated, coated or imprinted with a biomolecule or synthetic mimetic agent, e.g., CELLSTART™, MESENCULT™ ACF-substrate, ornithine, or polylysine, or an extracellular matrix protein, e.g., collagen, laminin, fibronectin, vitronectin, or the like.

The amnion derived adherent cells provided herein, and populations of such cells, can be isolated from one or more placentas. Isolated amnion derived cells can be cultured and expanded to produce populations of such cells. Populations of cells comprising amnion derived adherent cells can also be cultured and expanded to produce populations of amnion derived adherent cells.

In certain embodiments, AMDACs displaying any of the above marker and/or gene expression characteristics have been passaged at least 1, 2, 3, 4, 5, 6, 7, 8, 9, 10, 11, 12, 13, 14, 15, 16, 17, 18, 19 or 20 times, or more. In certain other embodiments, AMDACs displaying any of the above marker and/or gene expression characteristics have been doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

In a specific embodiment, the AMDACs described herein are telomerase⁻, as measured by RT-PCR and/or TRAP assays. In another specific embodiment, the AMDACs described herein do not express mRNA for telomerase reverse transcriptase (TERT) as determinable by RT-PCR, e.g., for 30 cycles. In another specific embodiment, the AMDACs described herein are NANOG⁻, as measured by RT-PCR. In another specific embodiment, the AMDACs described herein do not express mRNA for NANOG as determinable by RT-PCR, e.g., for 30 cycles. In a specific embodiment, the AMDACs described herein are (sex determining region Y)-box 2 (SOX2)⁻. In another specific embodiment, the AMDACs described herein do not express mRNA for SOX2 as determinable by RT-PCR, e.g., for 30 cycles. In another specific embodiment, the AMDACs described herein are not osteogenic as measured by an osteogenic phenotype assay (see, e.g., Section 5.12.2, below). In another specific embodiment, the AMDACs described herein are not chondrogenic as measured by a chondrogenic potential assay (see, e.g., Section 5.12.3, below). In another specific embodiment, the AMDACs described herein are not osteogenic as measured by an osteogenic phenotype assay (see, e.g., Section 5.12.2, below) and are not chondrogenic as measured by a chondrogenic potential assay (see, e.g., Section 5.12.3, below).

AMDACs can exhibit one or more of the characteristics described herein as determinable by RT-PCR, as demonstrated in Table 1. For example, AMDACs can exhibit one or more of such characteristics when isolated and cultured as described in Section 5.6, below.

TABLE 1

| AMDAC Marker | Positive | Negative |
|---|---|---|
| ACTA2 | X | |
| ACTC1 | X | |
| ADAMTS1 | X | |
| AMOT | X | |
| ANG | X | |
| ANGPT1 | X | |
| ANGPT2 | X | |
| ANGPT4 | | X |
| ANGPTL1 | X | |
| ANGPTL2 | X | |
| ANGPTL3 | | X |
| ANGPTL4 | X | |
| BAI1 | X | |
| BGLAP | | X |
| c-myc | X | |
| CD31 | | X |
| CD34 | | X |
| CD44 | X | |
| CD140a | X | |
| CD140b | X | |
| CD200 | X | |
| CD202b | X | |
| CD304 | X | |
| CD309 | X | |
| (VEGFR2/KDR) | | |
| CDH5 | | X |
| CEACAM1 | X | |
| CHGA | X | |
| COL15A1 | X | |
| COL18A1 | X | |
| COL4A1 | X | |
| COL4A2 | X | |
| COL4A3 | X | |
| Connexin-43 | X | |
| CSF3 | X | |
| CTGF | X | |
| CXCL10 | | X |
| CXCL12 | X | |
| CXCL2 | X | |
| DLX5 | | X |
| DNMT3B | X | |
| ECGF1 | X | |
| EDG1 | X | |
| EDIL3 | X | |
| ENPP2 | X | |
| EPHB2 | X | |
| F2 | X | |
| FBLN5 | X | |
| FGA | | X |
| FGF1 | X | |
| FGF2 | X | |
| FGF4 | | X |
| FIGF | X | |
| FLT3 | | X |
| FLT4 | X | |
| FN1 | X | |
| FOXC2 | X | |
| Follistatin | X | |
| Galectin-1 | X | |
| GRN | X | |
| HEY1 | X | |
| HGF | X | |
| HLA-G | | X |
| HSPG2 | X | |
| IFNB1 | X | |
| IFNG | | X |
| IL-8 | X | |
| IL-12A | X | |
| ITGA4 | X | |
| ITGAV | X | |
| ITGB3 | X | |
| KLF-4 | X | |
| LECT1 | | X |
| LEP | | X |
| MDK | X | |
| MMP-13 | | X |
| MMP-2 | X | |
| MYOZ2 | X | |
| NANOG | | X |
| NESTIN | | X |
| NRP2 | X | |
| PDGFB | X | |
| PF4 | X | |
| PGK1 | X | |
| PLG | | X |
| POU5F1 (OCT-4) | | X |
| PRL | | X |
| PROK1 | | X |
| PROX1 | X | |
| PTN | X | |
| SEMA3F | X | |
| SERPINB5 | X | |
| SERPINC1 | X | |
| SERPINF1 | X | |
| SOX2 | | X |
| TERT | | X |
| TGFA | X | |
| TGFB1 | X | |
| THBS1 | X | |
| THBS2 | X | |
| TIE1 | X | |
| TIMP2 | X | |
| TIMP3 | X | |
| TNF | X | |
| TNFSF15 | X | |
| TNMD | | X |
| TNNC1 | X | |
| TNNT2 | X | |
| VASH1 | X | |
| VEGF | X | |
| VEGFB | X | |
| VEGFC | X | |
| VEGFR1/FLT-1 | X | |
| XLKD1 | | X |

AMDACs can exhibit one or more of the characteristics described herein as determinable by immunolocalization, e.g., flow cytometry, as demonstrated in Table 2. For example, AMDACs can exhibit one or more of such characteristics when isolated and cultured as described in Section 5.6, below.

TABLE 2

| AMDAC Marker | Positive | Negative |
|---|---|---|
| CD6 | | X |
| CD9 | X | |
| CD10 | X | |
| CD31 | | X |
| CD34 | | X |
| CD44 | X | |
| CD45 | | X |
| CD49b | X | |
| CD49c | X | |
| CD49d | X | |
| CD54 | X | |
| CD68 | X | |
| CD90 | X | |
| CD98 | X | |
| CD105 | X | |
| CD117 | | X |
| CD133 | | X |
| CD143 | | X |
| CD144 | | X |
| (VE-cadherin) | | |
| CD146 | | X |
| CD166 | X | |
| CD184 | | X |
| CD200 | X | |
| CD202b | X | |
| CD271 | | X |
| CD304 | X | |
| CD309 | X | |
| (VEGFR2/KDR) | | |
| CD318 | X | |
| CD349 | X | |
| CytoK | X | |
| HLA-ABC+ B2 | X | |
| Micro+ | | |
| Invariant Chain+ | | X |
| HLA-DR-DP-DQ+ | | |
| PDL-1 | X | |
| VEGFR1/FLT-1 | X | |

AMDACs can exhibit one or more of the characteristics described herein as determinable by immunolocalization, e.g., immunofluorescence and/or immunohistochemistry, as demonstrated in Table 3. For example, AMDACs can exhibit one or more of such characteristics when isolated and cultured as described in Section 5.6, below.

TABLE 3

| AMDAC | | |
|---|---|---|
| Marker | Positive | Negative |
| CD31 | | X |
| CD34 | | X |
| (VEGFR2/KDR) | X | |
| Connexin-43 | X | |
| Galectin-1 | X | |
| TEM-7 | X | |

AMDACs can exhibit one or more of the characteristics described herein as determinable by immunolocalization, e.g., membrane proteomics, as demonstrated in Table 4. For example, AMDACs can exhibit one or more of such characteristics when isolated and cultured as described in Section 5.6, below.

TABLE 4

| AMDAC Marker | Positive | Negative |
|---|---|---|
| Activin receptor | X | |
| type IIB | | |
| ADAM 17 | X | |
| Alpha-actinin 1 | X | |
| Angiotensinogen | X | |
| Filamin A | X | |
| Macrophage | X | |
| acetylated LDL | | |
| receptor I and II | | |
| Megalin | X | |
| Myosin heavy | X | |
| chain non | | |
| muscle type A | | |
| Myosin-binding | X | |
| protein C cardiac | | |
| type | | |
| Wnt-9 | X | |

AMDACs can exhibit one or more of the characteristics described herein as determinable by secretome analysis, e.g., ELISA, as demonstrated in Table 5. For example, AMDACs can exhibit one or more of such characteristics when isolated and cultured as described in Section 5.6, below.

TABLE 5

| AMDAC Marker | Positive | Negative |
|---|---|---|
| ANG | X | |
| EGF | X | |
| ENA-78 | X | |
| FGF2 | X | |
| Follistatin | X | |
| G-CSF | X | |
| GRO | X | |
| HGF | X | |
| IL-6 | X | |
| IL-8 | X | |
| Leptin | X | |
| MCP-1 | X | |
| MCP-3 | X | |
| PDGFB | X | |
| PLGF | X | |
| Rantes | X | |
| TGFB1 | X | |
| Thrombopoietin | X | |
| TIMP1 | X | |
| TIMP2 | X | |
| uPAR | X | |
| VEGF | X | |
| VEGFD | X | |

5.4 Populations of Amnion Derived Adherent Cells Comprising Other Cell Types

The isolated cell populations comprising amnion derived adherent cells described herein can comprise a second type of cell, e.g., placental cells that are not amnion derived adherent cells, or, e.g., cells that are not placental cells. For example, an isolated population of amnion derived adherent cells can comprise, e.g., can be combined with, a population of a second type of cells, wherein said second type of cell are, e.g., embryonic stem cells, blood cells (e.g., placental blood, placental blood cells, umbilical cord blood, umbilical cord blood cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), placental stem cells (e.g., the placental stem cells described in U.S. Pat. No. 7,468,276, and in U.S. Patent Application Publication No. 2007/0275362, the disclosures of which are incorporated herein by reference in their entireties), nucleated cells from placental perfusate, e.g., total nucleated cells from placental perfusate, the cells described and claimed in U.S. Pat. No. 7,638,141, the disclosure of which is hereby incorporated by reference in its entirety, umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, and the like. In a specific embodiment, an isolated population of cells comprising amnion derived adherent cells comprises placental stem cells or stem cells from umbilical cord. In certain embodiments in which the second type of cell is blood or blood cells, erythrocytes have been removed from the population of cells.

In a specific embodiment, the second type of cell is a hematopoietic stem cell. Such hematopoietic stem cells can be, for example, contained within unprocessed placental blood, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

In a another embodiment, the second cell type is a non-embryonic cell type manipulated in culture in order to express markers of pluripotency and functions associated with embryonic stem cells

In specific embodiments of the above isolated populations of amnion derived adherent cells, either or both of the amnion derived adherent cells and cells of a second type are autologous, or are allogeneic, to an intended recipient of the cells.

Further provided herein is a composition comprising amnion derived adherent cells, and a plurality of stem cells other than the amnion derived adherent cells. In a specific embodiment, the composition comprises a stem cell that is obtained from a placenta, i.e., a placental stem cell, e.g., placental stem cells as described in U.S. Pat. Nos. 7,045,148; 7,255,879; and 7,311,905, and in U.S. Patent Application Publication No. 2007/0275362, the disclosures of each of which are incorporated herein by reference in their entireties. In a specific embodiment, the placental stem cells are CD34⁻, CD10⁺ and CD105⁺. In a more specific embodiment, the placental stem cells are CD34⁻, CD10⁺, CD105⁺ and CD200⁺. In a more specific embodiment, the placental stem cells are CD34⁻, CD45⁻, CD10⁺, CD90⁺, CD105⁺ and CD200⁺. In a more specific embodiment, the placental stem cells are CD34⁻, CD45⁻, CD80⁻, CD86⁻, CD10⁺, CD90⁺, CD105⁺ and CD200⁺. In other specific embodiments, said placental stem cells are CD200⁺ and HLA-G⁺; CD73⁺, CD105⁺, and CD200⁺; CD200⁺ and OCT-4⁺; CD73⁺, CD105⁺ and HLA-G⁺; CD73⁺ and CD105⁺ and facilitate the formation of one or more embryoid-like bodies in a population of placental cells comprising said stem cell when said population is cultured under conditions that allow the formation of an embryoid-like body; or OCT-4⁺ and facilitate the formation of one or more embryoid-like bodies in a population of placental cells comprising the stem cell when said population is cultured under conditions that allow formation of embryoid-like bodies; or any combination thereof. In a more specific embodiment, said CD200⁺, HLA-G⁺ stem cells are CD34⁻, CD38⁻, CD45⁻, CD73⁺ and CD105⁺. In another more specific embodiment, said CD73⁺, CD105⁺, and CD200⁺ stem cells are CD34⁻, CD38⁻, CD45⁻, and HLA-G⁺. In another more specific embodiment, said CD200⁺, OCT-4⁺ stem cells are CD34⁻, CD38⁻, CD45⁻, CD73⁺, CD105⁺ and HLA-G⁺. In another more specific embodiment, said CD73⁺, CD105⁺ and HLA-G⁺ stem cells are CD34⁻, CD45⁻, OCT-4⁺ and CD200⁺. In another more specific embodiment, said CD73⁺ and CD105⁺ stem cells are OCT-4⁺, CD34⁻, CD38⁻ and CD45⁻. In another more specific embodiment, said OCT-4⁺ stem cells are CD73⁺, CD105⁺, CD200⁺, CD34⁻, CD38⁻, and CD45⁻. In another more specific embodiment, the placental stem cells are maternal in origin (that is, have the maternal genotype). In another more specific embodiment, the placental stem cells are fetal in origin (that is, have the fetal genotype).

In another specific embodiment, the composition comprises amnion derived adherent cells, and embryonic stem cells. In another specific embodiment, the composition comprises amnion derived adherent cells and mesenchymal stromal or stem cells, e.g., bone marrow-derived mesenchymal stromal or stem cells. In another specific embodiment, the composition comprises bone marrow-derived hematopoietic stem cells. In another specific embodiment, the composition comprises amnion derived adherent cells and hematopoietic progenitor cells, e.g., hematopoietic progenitor cells from bone marrow, fetal blood, umbilical cord blood, placental blood, and/or peripheral blood. In another specific embodiment, the composition comprises amnion derived adherent cells and somatic stem cells. In a more specific embodiment, said somatic stem cell is a neural stem cell, a hepatic stem cell, a pancreatic stem cell, an endothelial stem cell, a cardiac stem cell, or a muscle stem cell.

In other specific embodiments, the second type of cells comprise about, at least, or no more than, 10%, 15%, 20%, 25%, 30%, 35%, 40%, 45%, or 50% of cells in said population. In other specific embodiments, the AMDACs in said composition comprise at least 50%, 55%, 60%, 65%, 70%, 75%, 80%, 85% or 90% of cells in said composition. In other specific embodiments, the amnion derived adherent cells comprise about, at least, or no more than, 10%, 15%, 20%, 25%, 30%, 35%, 40%, or 45% of cells in said population. In other specific embodiments, at least 25%, 35%, 45%, 50%, 60%, 75%, 85% or more of the cells in said population are not OCT-4⁺.

Cells in an isolated population of amnion derived adherent cells can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated population of amnion derived adherent cells can be combined with a plurality of cells of a plurality of cell types, as well.

5.5 Growth in Culture

The growth of the amnion derived adherent cells described herein, as for any mammalian cell, depends in part upon the particular medium selected for growth. Under optimum conditions, amnion derived adherent cells typically double in number in approximately 24 hours. During culture, the amnion derived adherent cells described herein adhere to a substrate in culture, e.g. the surface of a tissue culture container (e.g., tissue culture dish plastic, fibronectin-coated plastic, and the like) and form a monolayer. Typically, the cells establish in culture within 2-7 days after digestion of the amnion. They proliferate at approximately 0.4 to 1.2 population doublings per day and can undergo at least 30 to 50 population doublings. The cells display a mesenchymal/fibroblastic cell-like phenotype during subconfluence and expansion, and a cuboidal/cobblestone-like appearance at confluence, and proliferation in culture is strongly contact-inhibited. Populations of amnion-derived adherent cells can form embryoid bodies during expansion in culture.

5.6 Methods of Obtaining Amnion-Derived Adherent Cells

The amnion derived adherent cells, and populations of cells comprising the amnion derived adherent cells, can be produced, e.g., isolated from other cells or cell populations, for example, through particular methods of digestion of amnion tissue, optionally followed by assessment of the resulting cells or cell population for the presence or absence of markers, or combinations of markers, characteristics of amnion derived adherent cells, or by obtaining amnion cells and selecting on the basis of markers characteristic of amnion derived adherent cells.

The amnion derived adherent cells, and isolated populations of cells comprising the amnion derived adherent cells, provided herein can be produced by, e.g., digestion of amnion tissue followed by selection for adherent cells. In one embodiment, for instance, isolated amnion derived adherent cells, or an isolated population of cells comprising amnion derived adherent cells, can be produced by (1) digesting amnion tissue with a first enzyme to dissociate cells from the epithelial layer of the amnion from cells from the mesenchymal layer of the amnion; (2) subsequently digesting the mesenchymal layer of the amnion with a second enzyme to form a single-cell suspension; (3) culturing cells in said single-cell suspension on a tissue culture surface, e.g., tissue culture plastic; and (4) selecting cells that adhere to said surface after a change of medium, thereby producing an isolated population of cells comprising amnion derived adherent cells. In a specific embodiment, said first enzyme is trypsin. In a more specific embodiment, said trypsin is used at a concentration of 0.25% trypsin (w/v), in 5-20, e.g., 10 milliliters solution per gram of amnion tissue to be digested. In another more specific embodiment, said digesting with trypsin is allowed to proceed for about 15 minutes at 37° C. and is repeated up to three times. In another specific embodiment, said second enzyme is collagenase. In a more specific embodiment, said collagenase is used at a concentration between 50 and 500 U/L in 5 mL per gram of amnion tissue to be digested. In another more specific embodiment, said digesting with collagenase is allowed to proceed for about 45-60 minutes at 37° C. In another specific embodiment, the single-cell suspension formed after digestion with collagenase is filtered through, e.g., a 75 μM-150 μM filter between step (2) and step (3). In another specific embodiment, said first enzyme is trypsin, and said second enzyme is collagenase.

An isolated population of cells comprising amnion derived adherent cells can, in another embodiment, be obtained by selecting cells from amnion, e.g., cells obtained by digesting amnion tissue as described elsewhere herein, that display one or more characteristics of an amnion derived adherent cell. In one embodiment, for example, a cell population is produced by a method comprising identifying amnion cells that are (a) negative for OCT-4, as determinable by RT-PCR, and (b) positive for one or more of VEGFR2/KDR, CD9, CD54, CD105, CD200, as determinable or selectable by immunolocalization, e.g., flow cytometry; and isolating said cells from other cells to form a cell population. In a specific embodiment, said amnion cells are additionally VE-cadherin⁻. In a specific embodiment, a cell population is produced by selecting placental cells that are (a) negative for OCT-4, as determinable by RT-PCR, and VE-cadherin, as determinable by immunolocalization, e.g., flow cytometry, and (b) positive for each of VEGFR2/KDR, CD9, CD54, CD105, CD200, as determinable by immunolocalization, e.g., flow cytometry; and isolating said cells from other cells to form a cell population. In certain embodiments, selection by immunolocalization, e.g., flow cytometry, is performed before selection by RT-PCR. In another specific embodiment, said selecting comprises selecting cells that do not express cellular marker CD34 after culture for 4 to 21 days in the presence of 1 to 100 ng/mL VEGF.

In another embodiment, for example, a cell population is produced by a method comprising selecting amnion cells that are adherent to tissue culture plastic and are OCT-4⁻, as determinable by RT-PCR, and VEGFR1/Flt-1⁺ and VEGFR2/KDR⁺, as determinable by immunolocalization, e.g., flow cytometry, and isolating said cells from other cells to form a cell population. In a specific embodiment, a cell population is produced by a method comprising selecting amnion cells that are OCT-4⁻, as determinable by RT-PCR, and VEGFR1/Flt-1⁺, VEGFR2/KDR⁺, and HLA-G⁻, as determinable by immunolocalization, e.g., flow cytometry. In another specific embodiment, said cell population is produced by selecting amnion cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and/or CXCR4⁻ (chemokine (C—X—C motif) receptor 4) as determinable by immunolocalization, e.g., flow cytometry, and isolating the cells from cells that do not display one or more of these characteristics. In another specific embodiment, said cell population is produced by selecting amnion cells that are additionally VE-cadherin⁻ as determinable by immunolocalization, e.g., flow cytometry, and isolating the cells from cells that are VE-cadherin⁺. In another specific embodiment, said cell population is produced by selecting amnion cells that are additionally CD105⁺ and CD200⁺ as determinable by immunolocalization, e.g., flow cytometry, and isolating the cells from cells that are CD105⁻ or CD200⁻. In another specific embodiment, said cell does not express CD34 as detected by immunolocalization, e.g., flow cytometry, after exposure to 1 to 100 ng/mL VEGF for 4 to 21 days.

In the selection of cells, it is not necessary to test an entire population of cells for characteristics specific to amnion derived adherent cells. Instead, one or more aliquots of cells (e.g., about 0.5%-2%) of a population of cells may be tested for such characteristics, and the results can be attributed to the remaining cells in the population.

Selected cells can be confirmed to be the amnion derived adherent cells provided herein by culturing a sample of the cells (e.g., about 10⁴ to about 10⁵ cells) on a substrate, e.g., MATRIGEL™, for 4 to 14, e.g., 7, days in the presence of VEGF (e.g., about 50 ng/mL), and visually inspecting the cells for the appearance of sprouts and/or cellular networks.

Amnion derived adherent cells can be selected by the above markers using any method known in the art of cell selection. For example, the adherent cells can be selected using an antibody or antibodies to one or more cell surface markers, for example, in immunolocalization, e.g., flow cytometry or FACS. Selection can be accomplished using antibodies in conjunction with magnetic beads. Antibodies that are specific for certain markers are known in the art and are available commercially, e.g., antibodies to CD9 (Abcam); CD54 (Abcam); CD105 (Abeam; BioDesign International, Saco, Me., etc.); CD200 (Abcam) cytokeratin (SigmaAldrich). Antibodies to other markers are also available commercially, e.g., CD34, CD38 and CD45 are available from, e.g., StemCell Technologies or BioDesign International. Primers to OCT-4 sequences suitable for RT-PCR can be obtained commercially, e.g., from Millipore or Invitrogen, or can be readily derived from the human sequence in GenBank Accession No. DQ486513.

Detailed methods of obtaining placenta and amnion tissue from placenta, and treating such tissue in order to obtain amnion derived adherent cells, are provided below.

5.6.1 Cell Collection Composition

Generally, cells can be obtained from amnion from a mammalian placenta, e.g., a human placenta, using a physiologically-acceptable solution, e.g., a cell collection composition. In some embodiments, the cell collection composition prevents or suppresses apoptosis, prevents or suppresses cell death, lysis, decomposition and the like. A cell collection composition is described in detail in related U.S. Patent Application Publication No. 2007/0190042, entitled “Improved Medium for Collecting Placental Stem Cells and Preserving Organs,” the disclosure of which is incorporated herein by reference in its entirety.

The cell collection composition can comprise any physiologically-acceptable solution suitable for the collection and/or culture of amnion derived adherent cells, for example, a saline solution (e.g., phosphate-buffered saline, Kreb's solution, modified Kreb's solution, Eagle's solution, 0.9% NaCl, etc.), a culture medium (e.g., DMEM, H.DMEM, etc.), and the like, with or without the addition of a buffering component, e.g., 4-(2-hydroxyethyl)-1-piperazineethanesulfonic acid (HEPES).

The cell collection composition can comprise one or more components that tend to preserve cells, e.g., amnion derived adherent cells, that is, prevent the cells from dying, or delay the death of the cells, reduce the number of cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE™, hyaluronidase, and the like. The use of cell collection compositions comprising tissue-digesting enzymes is discussed in more detail below.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g. butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/l to about 100,000 units/l); or an amiloride containing compound (e.g., amiloride, ethyl isopropyl amiloride, hexamethylene amiloride, dimethyl amiloride or isobutyl amiloride present at about 1.0 μM to about 5 μM).

The amnion derived adherent cells described herein can also be collected, e.g., during and after digestion as described below, into a simple physiologically-acceptable buffer, e.g., phosphate-buffered saline, a 0.9% NaCl solution, cell culture medium, or the like.

5.6.2 Collection and Handling of Placenta

Generally, a human placenta is recovered shortly after its expulsion after birth, or after, e.g., Caesarian section. In a preferred embodiment, the placenta is recovered from a patient after informed consent and after a complete medical history of the patient is obtained and is associated with the placenta. Preferably, the medical history continues after delivery. Such a medical history can be used to coordinate subsequent use of the placenta or cells harvested therefrom. For example, human placental cells, e.g., amnion derived adherent cells, can be used, in light of the medical history, for personalized medicine for the infant, or a close relative, associated with the placenta, or for parents, siblings, or other relatives of the infant.

Prior to recovery of amnion derived adherent cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta. The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of cells by, e.g., tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to cell collection, can be stored under sterile conditions and at a temperature of, e.g., 4 to 25° C. (centigrade), e.g., at room temperature. The placenta may be stored for, e.g., a period of for zero to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta can be stored in an anticoagulant solution at a temperature of, e.g., 4 to 25° C. (centigrade). Suitable anticoagulant solutions are well known in the art. For example, a solution of sodium citrate, heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before cells are collected.

See, e.g., U.S. Pat. No. 7,638,141, the disclosure of which is hereby incorporated by reference in its entirety, for additional information regarding collection and handling of placenta.

5.6.3 Physical Disruption and Enzymatic Digestion of Amnion Tissue

In one embodiment, the amnion is separated from the rest of the placenta, e.g., by blunt dissection, e.g., using the fingers. The amnion can be dissected, e.g., into parts or tissue segments, prior to enzymatic digestion and adherent cell recovery. Amnion derived adherent cells can be obtained from a whole amnion, or from a small segment of amnion, e.g., a segment of amnion that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 square millimeters in area.

Amnion derived adherent cells can generally be collected from a placental amnion or a portion thereof, at any time within about the first three days post-expulsion, but preferably between about 0 hours and 48 hours after expulsion, or about 8 hours and about 18 hours post-expulsion.

AMDACs can, for example, be isolated using a specific two-step isolation method comprising digestion with trypsin followed by digestion with collagenase. For example, provided herein is a method of isolating amnion derived adherent cells comprising digesting an amniotic membrane or portion thereof with trypsin such that epithelial cells are released from said amniotic membrane; removing the amniotic membrane or portion thereof from said epithelial cells; further digesting the amniotic membrane or portion thereof with collagenase. In a specific embodiment, digestion of the amniotic membrane or portion thereof with trypsin is repeated at least once. In another specific embodiment, digestion of the amniotic membrane or portion thereof with collagenase is repeated at least once. In another specific embodiment, the trypsin is at about 0.1%-1.0% (final concentration). In a more specific embodiment, the trypsin is at about 0.25% (final concentration). In another specific embodiment, the collagenase is at about 50 U/mL to about 1000 U/mL (final concentration). In a more specific embodiment, the collagenase is at about 125 U/mL (final concentration).

In one embodiment, for example, amnion derived adherent cells can be obtained as follows. The amniotic membrane is isolated from the placenta via, e.g., blunt dissection, then cut into segments approximately 0.1″×0.1″ to about 5″×5″, e.g., 2″×2″, in size. The epithelial monolayer is removed from the fetal side of the amniotic membrane by trypsinization, e.g., triple trypsinization as follows. The segments of amniotic membrane are placed into a container with warm (e.g., about 20° C. to about 37° C.) trypsin-EDTA solution (0.25%). The volume of the trypsin solution can range from about 5 mL per gram of amniotic membrane to about 50 mL per gram of amniotic membrane. The container is agitated for about 5 minutes to about 30 minutes, e.g., 15 minutes, while maintaining the temperature constant. The segments of amniotic membrane are then separated from the trypsin solution by any appropriate method, such as manually removing the amnion segments, or by filtration. The trypsinization step can be repeated at least one more time. In one embodiment, the trypsinization step is repeated twice (for triple trypsinization) or three times (for quadruple trypsinization).

In one embodiment, upon completion of the final trypsinization, the segments of amniotic membrane are placed into warm (e.g., about 20° C. to about 37° C.) trypsin neutralization solution (e.g., at a volume of about 5 mL per gram of amniotic membrane to about 50 mL per gram of amniotic membrane), such as phosphate-buffered saline (PBS)/10% fetal bovine serum (FBS), PBS/5% FBS or PBS/3% FBS, and agitated for about 5 seconds to about 30 minutes, e.g., 5, 10, or 15 minutes. The segments of amniotic membrane are then separated from the trypsin neutralization solution by any appropriate method, such as manually removing the amnion segments, or by filtration. The segments of amniotic membrane are then placed into a warm (e.g., about 20° C. to about 37° C.) PBS, pH 7.2, solution (e.g., at a volume of about 5 mL per gram of amniotic membrane to about 50 mL per gram of amniotic membrane), agitated for about 5 seconds to about 30 minutes, e.g., 5, 10, or 15 minutes. The amniotic membrane segments are then separated from the PBS as described above.

The segments of amniotic membrane are then placed into warm (e.g., about 20° C. to about 37° C.) digestion solution. The volume of digestion solution can range from about 5 mL per gram of amnion to about 50 mL per gram of amnion. Digestion solutions comprise digestion enzymes in an appropriate culture medium, such as DMEM. Typical digestion solutions include collagenase type I (about 50 U/mL to about 500 U/mL). Digestion solutions for this stage of the process do not generally comprise trypsin. Agitation is generally at 37° C. until amnion digestion is substantially complete as evidenced by, e.g., complete dissolution of the amniotic membrane yielding a homogenous suspension (approximately 10 minutes to about 90 minutes). Warm PBS/5% FBS is then added at a ratio of about 1 mL per gram of amniotic tissue to about 50 mL per gram of amniotic tissue and agitated for about 2 minutes to about 5 minutes. The cell suspension is then filtered to remove any un-digested tissue using, e.g., a 40 μm to 100 μm filter. The cells are suspended in warm PBS (about 1 mL to about 500 mL), and then centrifuged at 200×g to about 400×g for about 5 minutes to about 30 minutes, e.g. 300×g for about 15 minutes at 20° C. After centrifugation, the supernatant is removed and the cells are resuspended in a suitable culture medium. The cell suspension can be filtered (40 μm to 70 μm filter) to remove any remaining undigested tissue, yielding a single cell suspension. The remaining undigested amnion, in this embodiment, can be discarded.

In this embodiment, cells in suspension are collected and cultured as described elsewhere herein to produce isolated amnion derived adherent cells, and populations of such cells. For example, in one embodiment, the cells in suspension can be cultured and amnion derived adherent cells can be separated from non-adherent cells in said culture to produce an enriched population of amnion derived adherent cells. In more specific embodiments, at least 50%, 55%, 60%, 65%, 70%, 75%, 80%, 85%, 90%, 95%, 98% or 99% of cells in said enriched population of amnion derived adherent cells are said amnion derived adherent cells.

In any of the digestion protocols herein, the cell suspension obtained by digestion can be filtered, e.g., through a filter comprising pores from about 50 μm to about 150 μm, e.g., from about 75 μm to about 125 μm. In a more specific embodiment, the cell suspension can be filtered through two or more filters, e.g., a 125 μm filter and a 75 μm filter.

In conjunction with any of the methods described herein, AMDACs can be isolated from the cells released during digestion by selecting cells that express one or more characteristics of AMDACs, as described in Section 5.3, above.

In one embodiment, AMDACs can be isolated using, in order, a first enzyme and a second enzyme, wherein the first enzyme used in the method is not collagenase, and wherein the second enzyme used in the method is not trypsin.

In another embodiment, the digestion step used to isolate AMDACs does not use a combination of any two or more of collagenase, dispase or hyaluronidase.

In another embodiment, the AMDACs are not isolated via explant culturing to allow the cells to be detected by growth, replication, or migration out of the explants.

In another embodiment, deoxyribonuclease (DNase) is not used during the isolation of AMDACs. For example, DNase is not used following the collagenase digestion step of the isolation.

5.6.4 Isolation, Sorting, and Characterization of Amnion Derived Adherent Cells

Cell pellets can be resuspended in fresh cell collection composition, as described above, or a medium suitable for cell maintenance, e.g., Dulbecco's Modified Eagle's Medium (DMEM); Iscove's Modified Dulbecco's Medium (IMDM), e.g. IMDM serum-free medium containing 2 U/mL heparin and 2 mM EDTA (GibcoBRL, NY); a mixture of buffer (e.g. PBS, HBSS) with FBS (e.g. 2% v/v); or the like.

Amnion derived adherent cells that have been cultured, e.g., on a surface, e.g., on tissue culture plastic, with or without additional extracellular matrix coating such as fibronectin, can be passaged or isolated by differential adherence. For example, a cell suspension obtained as described in Section 5.6.3, above, can be cultured, e.g., for 3-7 days in culture medium on tissue culture plastic. During culture, a plurality of cells in the suspension adhere to the culture surface and nonadherent cells are removed during medium exchange.

The number and type of cells collected from amnion can be monitored, for example, by measuring changes in morphology and cell surface markers using standard cell detection techniques such as immunolocalization, e.g., flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using one or more antibodies to CD34, one can determine, using the techniques above, whether a cell comprises a detectable amount of CD34; if so, the cell is CD34⁺.

Amnion derived adherent cells can be isolated by Ficoll separation, e.g., Ficoll gradient centrifugation. Such centrifugation can follow any standard protocol for centrifugation speed, etc. In one embodiment, for example, cells recovered after digestion of the amnion are separated using a Ficoll gradient by centrifugation at 5000×g for 15 minutes at room temperature and cell layers of interest are collected for further processing.

Amnion-derived cells, e.g., cells that have been isolated by Ficoll separation, differential adherence, or a combination of both, can be sorted using a fluorescence activated cell sorter (FACS). Fluorescence activated cell sorting (FACS) is a well-known method for separating particles, including cells, based on the fluorescent properties of the particles (see, e.g., Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, cells from placenta, e.g., amnion derived adherent cells, can be sorted on the basis of expression of the markers CD49f, VEGFR2/KDR, and/or Flt-1/VEGFR1. Preferably the cells are identified as being OCT-4⁻, e.g., by determining the expression of OCT-4 by RT-PCR in a sample of the cells, wherein the cells are OCT-4⁻ if the cells in the sample fail to show detectable production of mRNA for OCT-4 after 30 cycles. For example, cells from amnion that are VEGFR2/KDR⁺ and VEGFR1/Flt-1⁺ can be sorted from cells that are one or more of VEGFR2/KDR⁻, and VEGFR1/Flt-1⁺, CD9⁺, CD54⁺, CD105⁺, CD200⁺, and/or VE-cadherin⁻. In a specific embodiment, amnion-derived, tissue culture plastic-adherent cells that are one or more of CD49f⁺, VEGFR2/KDR⁺, CD9⁺, CD54⁺, CD105⁺, CD200⁺, and/or VE-cadherin⁻, or cells that are VEGFR2/KDR⁺, CD9⁺, CD54⁺, CD105⁺, CD200⁺, and VE-cadherin⁻, are sorted away from cells not expressing one or more of such marker(s), and selected. In another specific embodiment, CD49f⁺, VEGFR2/KDR⁺, VEGFR1/Flt-1⁺ cells that are additionally one or more, or all, of CD31⁺ CD34⁺, CD45⁺, CD133⁻, and/or Tie-2⁺ are sorted from cells that do not display one or more, or any, of such characteristics. In another specific embodiment, VEGFR2/KDR⁺, VEGFR1/Flt-1⁺ cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and/or CXCR4⁻, are sorted from cells that do not display one or more, or any, of such characteristics.

Selection for amnion derived adherent cells can be performed on a cell suspension resulting from digestion, or on isolated cells collected from digestate, e.g., by centrifugation or separation using flow cytometry. Selection by expressed markers can be accomplished alone or, e.g., in connection with procedures to select cells on the basis of their adherence properties in culture. For example, an adherence selection can be accomplished before or after sorting on the basis of marker expression.

With respect to antibody-mediated detection and sorting of placental cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against CD105 (available from R&D Systems Inc., Minneapolis, Minn.); phycoerythrin (PE) conjugated monoclonal antibodies against CD200 (BD Biosciences Pharmingen); VEGFR2/KDR-Biotin (CD309, Abcam), and the like. Antibodies to any of the markers disclosed herein can be labeled with any standard label for antibodies that facilitates detection of the antibodies, including, e.g., horseradish peroxidase, alkaline phosphatase, β-galactosidase, acetylcholinesterase streptavidin/biotin, avidin/biotin, umbelliferone, fluorescein, fluorescein isothiocyanate (FITC), rhodamine, dichlorotriazinylamine fluorescein, dansyl chloride or phycoerythrin (PE), luminol, luciferase, luciferin, and aequorin, and examples of suitable radioactive material include ¹²⁵I, ¹³¹I, ³⁵S or ³H.

Amnion derived adherent cells can be labeled with an antibody to a single marker and detected and/sorted based on the single marker, or can be simultaneously labeled with multiple antibodies to a plurality of different markers and sorted based on the plurality of markers.

In another embodiment, magnetic beads can be used to separate cells, e.g., to separate the amnion derived adherent cells described herein from other amnion cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Amnion derived adherent cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay or MTT cell proliferation assay (to assess proliferation). Longevity may be determined by methods well known in the art, such as by determining the maximum number of population doubling in an extended culture.

5.7 Culture of Amnion Derived Adherent Cells

5.7.1 Culture Media

Isolated amnion derived adherent cells, or populations of such cells, can be used to initiate, or seed, cell cultures. Cells are generally transferred to sterile tissue culture vessels either uncoated or coated with extracellular matrix or biomolecules such as laminin, collagen (e.g., native or denatured), gelatin, fibronectin, ornithine, vitronectin, and extracellular membrane protein (e.g., MATRIGEL™ (BD Discovery Labware, Bedford, Mass.)).

AMDACs can, for example, be established in media suitable for the culture of stem cells. Establishment media can, for example, include EGM-2 medium (Lonza), DMEM+10% FBS, or medium comprising 60% DMEM-LG (Gibco), 40% MCDB-201 (Sigma), 2% fetal calf serum (FCS) (Hyclone Laboratories), 1× insulin-transferrin-selenium (ITS), 1× lenoleic-acid-bovine-serum-albumin (LA-BSA), 10⁻⁹ M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin (referred to herein as “standard medium”).

Amnion derived adherent cells can be cultured in any medium, and under any conditions, recognized in the art as acceptable for the culture of cells, e.g., adherent placental stem cells. Preferably, the culture medium comprises serum. In various embodiments, media for the culture or subculture of AMDACs includes STEMPRO® (Invitrogen), MSCM-sf (ScienCell, Carlsbad, Calif.), MESENCULT®-ACF medium (StemCell Technologies, Vancouver, Canada), standard medium, standard medium lacking EGF, standard medium lacking PDGF, DMEM+10% FBS, EGM-2 (Lonza), EGM-2MV (Lonza), 2%, 10% and 20% ES media, ES-SSR medium, or α-MEM-20% FBS. Medium acceptable for the culture of amnion derived adherent cells includes, e.g., DMEM, IMDM, DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM Lonza), ADVANCESTEM™ Medium (Hyclone), KNOCKOUT™ DMEM (Invitrogen), Leibovitz's L-15 medium, MCDB, DMEM/F12, RPMI 1640, advanced DMEM (Gibco), DMEM/MCDB201 (Sigma), and CELL-GRO FREE, or the like. In various embodiments, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising about 2 to about 20%, e.g., about 10%, fetal bovine serum (FBS; e.g. defined fetal bovine serum, Hyclone, Logan Utah); DMEM-HG comprising about 2 to about 20%, e.g., about 15%, FBS; IMDM (Iscove's modified Dulbecco's medium) comprising about 2 to about 20%, e.g., about 10%, FBS, about 2 to about 20%, e.g., about 10%, horse serum, and hydrocortisone; M199 comprising about 2 to about 20%, e.g., about 10%, FBS, EGF, and heparin; α-MEM (minimal essential medium) comprising about 2 to about 20%, e.g., about 10%, FBS, GLUTAMAX™ and gentamicin; DMEM comprising 10% FBS, GLUTAMAX™ and gentamicin; DMEM-LG comprising about 2 to about 20%, e.g., about 15%, (v/v) fetal bovine serum (e.g., defined fetal bovine serum, Hyclone, Logan Utah), antibiotics/antimycotics (e.g., penicillin at about 100 Units/milliliter, streptomycin at 100 micrograms/milliliter, and/or amphotericin B at 0.25 micrograms/milliliter (Invitrogen, Carlsbad, Calif.)), and 0.001% (v/v) β-mercaptoethanol (Sigma, St. Louis Mo.); KNOCKOUT™-DMEM basal medium supplemented with 2 to 20% FBS, non-essential amino acid (Invitrogen), beta-mercaptoethanol, KNOCKOUT™ basal medium supplemented with KNOCKOUT™ Serum Replacement, alpha-MEM comprising 2 to 20% FBS, EBM2™ basal medium supplemented with EGF, VEGF, bFGF, R3-IGF-1, hydrocortisone, heparin, ascorbic acid, FBS, gentamicin), or the like.

The culture medium can be supplemented with one or more components including, for example, serum (e.g., FCS or FBS, e.g., about 2-20% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

Amnion derived adherent cells (AMDACs) can be cultured in standard tissue culture conditions, e.g., in tissue culture dishes or multiwell plates. The cells can also be cultured using a hanging drop method. In this method, the cells are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the cells are cultured. AMDACs can also be cultured in standard or high-volume or high-throughput culture systems, such as T-flasks, Corning HYPERFLASK®, Cell Factories (Nunc), 1-, 2-, 4-, 10 or 40-Tray Cell stacks, and the like.

In one embodiment, amnion derived adherent cells are cultured in the presence of a compound that acts to maintain an undifferentiated phenotype in the cells. In a specific embodiment, the compound is a substituted 3,4-dihydropyridimol[4,5-d]pyrimidine. In a more specific embodiment, the compound is a compound having the following chemical structure:

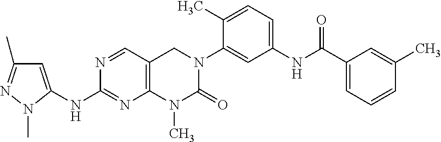

The compound can be contacted with an amnion derived adherent cell, or population of such cells, at a concentration of, for example, between about 1 μM to about 10 μM.

5.7.2 Expansion and Proliferation of Amnion Derived Adherent Cells

Once an isolated amnion derived adherent cell, or isolated population of such cells (e.g., amnion derived adherent cells, or population of such cells separated from at least 50% of the amnion cells with which the cell or population of cells is normally associated in vivo), the cells can be proliferated and expanded in vitro. For example, a population of adherent cells or amnion derived adherent cells can be cultured in tissue culture containers, e.g., dishes, flasks, multiwell plates, or the like, for a sufficient time for the cells to proliferate to 40-70% confluence, that is, until the cells and their progeny occupy 40-70% of the culturing surface area of the tissue culture container.

Amnion derived adherent cells can be seeded in culture vessels at a density that allows cell growth. For example, the cells may be seeded at low density (e.g., about 400 to about 6,000 cells/cm²) to high density (e.g., about 20,000 or more cells/cm²). In a preferred embodiment, the cells are cultured at about 0% to about 5% by volume CO₂ in air. In some preferred embodiments, the cells are cultured at about 0.1% to about 25% O₂ in air, preferably about 5% to about 20% O₂ in air. The cells are preferably cultured at about 25° C. to about 40° C., preferably at about 37° C.

The cells are preferably cultured in an incubator. During culture, the culture medium can be static or can be agitated, for example, during culture using a bioreactor. Amnion derived adherent cells preferably are grown under low oxidative stress (e.g., with addition of glutathione, ascorbic acid, catalase, tocopherol, N-acetylcysteine, or the like).

Although the cells may be grown to confluence, the cells are preferably not grown to confluence. For example, once 40%-70% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 cells, preferably about 50,000 cells, or about 400 to about 6,000 cells/cm², can be passaged to a new culture container containing fresh culture medium. Typically, the new medium is the same type of medium from which the cells were removed. The amnion derived adherent cells can be passaged at least 1, 2, 3, 4, 5, 6, 7, 8, 9, 10, 11, 12, 13, 14, 15, 16, 17, 18, 19 or 20 times, or more. AMDACs can be doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

5.8 Compositions Comprising Amnion Derived Adherent Cells

5.8.1 Pharmaceutical Compositions Comprising Amnion Derived Adherent Cells

In certain embodiments, amnion derived adherent cells are contained within, or are components of, a pharmaceutical composition. The cells can be prepared in a form that is easily administrable to an individual, e.g., amnion derived adherent cells that are contained within a container that is suitable for medical use. Such a container can be, for example, a syringe, sterile plastic bag, vial, flask, jar, or other container from which the amnion derived adherent cell population can be easily dispensed. For example, the container can be a blood bag or other plastic, medically-acceptable bag suitable for the intravenous administration of a liquid to a recipient. The container, in certain embodiments, is one that allows for cryopreservation of the cells. The cells in the compositions, e.g., pharmaceutical compositions, provided herein, can comprise amnion derived adherent cells derived from a single donor, or from multiple donors. The cells can be completely HLA-matched to an intended recipient, or partially or completely HLA-mismatched.

Thus, in one embodiment, amnion derived adherent cells in the compositions provided herein are administered to an individual in need thereof in the form of a composition comprising amnion derived adherent cells in a container. In another specific embodiment, the container is a bag, flask, vial, or jar. In more specific embodiment, said bag is a sterile plastic bag. In a more specific embodiment, said bag is suitable for, allows or facilitates intravenous administration of said adherent cells, e.g., by intravenous infusion, bolus injection, or the like. The bag can comprise multiple lumens or compartments that are interconnected to allow mixing of the cells and one or more other solutions, e.g., a drug, prior to, or during, administration. In another specific embodiment, prior to cryopreservation, the solution comprising the amnion derived adherent cells comprises one or more compounds that facilitate cryopreservation of the cells. In another specific embodiment, said amnion derived adherent cells are contained within a physiologically-acceptable aqueous solution. In a more specific embodiment, said physiologically-acceptable aqueous solution is a 0.9% NaCl solution. In another specific embodiment, said amnion derived adherent cells comprise cells that are HLA-matched to a recipient of said cells. In another specific embodiment, said amnion derived adherent cells comprise cells that are at least partially HLA-mismatched to a recipient of said cells. In another specific embodiment, said amnion derived adherent cells are derived from a plurality of donors. In various specific embodiments, said container comprises about, at least, or at most 1×10⁶ said cells, 5×10⁶ said cells, 1×10⁷ said stem cells, 5×10⁷ said cells, 1×10⁸ said cells, 5×10⁸ said cells, 1×10⁹ said cells, 5×10⁹ said cells, 1×10¹⁰, or 1×10¹¹ said cells. In other specific embodiments of any of the foregoing cryopreserved populations, said cells have been passaged about, at least, or no more than 5 times, no more than 10 times, no more than 15 times, or no more than 20 times. In another specific embodiment of any of the foregoing cryopreserved cells, said cells have been expanded within said container. In specific embodiments, a single unit dose of amnion derived adherent cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more amnion derived adherent cells.

In certain embodiments, the pharmaceutical compositions provided herein comprises populations of amnion derived adherent cells, that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

5.8.2 Matrices Comprising Amnion Derived Adherent Cells

Further provided herein are compositions comprising matrices, hydrogels, scaffolds, and the like. Such compositions can be used in the place of, or in addition to, such cells in liquid suspension.

The matrix can be, e.g., a permanent or degradable decellularized tissue, e.g., a decellularized amniotic membrane, or a synthetic matrix. The matrix can be a three-dimensional scaffold. In a more specific embodiment, said matrix comprises collagen, gelatin, laminin, fibronectin, pectin, ornithine, or vitronectin. In another more specific embodiment, the matrix is an amniotic membrane or an amniotic membrane-derived biomaterial. In another more specific embodiment, said matrix comprises an extracellular membrane protein. In another more specific embodiment, said matrix comprises a synthetic compound. In another more specific embodiment, said matrix comprises a bioactive compound. In another more specific embodiment, said bioactive compound is a growth factor, a cytokine, an antibody, or an organic molecule of less than 5,000 daltons.

The amnion derived adherent cells described herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which the amnion derived adherent cells provided herein can be seeded are described in Hariri, U.S. Application Publication No. 2004/0048796, the disclosure of which is incorporated by reference herein in its entirety.

In another specific embodiment, the matrix is a composition comprising an extracellular matrix. In a more specific embodiment, said composition is MATRIGEL™ (BD Biosciences).

The isolated amnion derived adherent cells described herein can be suspended in a hydrogel solution suitable for, e.g., injection. The hydrogel is, e.g., an organic polymer (natural or synthetic) that is cross-linked via covalent, ionic, or hydrogen bonds to create a three-dimensional open-lattice structure that entraps water molecules to form a gel. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. The amnion derived adherent cells in such a matrix can also be cultured so that the cells are mitotically expanded, e.g., prior to implantation. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In certain embodiments, the compositions comprising cells, provided herein, comprise an in situ polymerizable gel (see, e.g., U.S. Patent Application Publication 2002/0022676; Anseth et al., J. Control Release, 78(1-3):199-209 (2002); Wang et al., Biomaterials, 24(22):3969-80 (2003). In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

In a specific embodiment, the matrix is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another preferred embodiment the cells of the invention are seeded onto foam scaffolds that may be composite structures. In addition, the three-dimensional framework may be molded into a useful shape, such as a specific structure in the body to be repaired, replaced, or augmented. Other examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

The amnion derived adherent cells described herein can be seeded onto a three-dimensional framework or scaffold and implanted in vivo. Such a framework can be implanted in combination with any one or more growth factors, cells, drugs or other components that, e.g., stimulate tissue formation, e.g., bone formation or formation of vasculature.

The amnion derived adherent cells provided herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the matrix comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The matrix can also comprise anti-thrombotic agents such as heparin; the scaffolds can also be treated to alter the surface charge (e.g., coating with plasma) prior to seeding with the adherent cells provided herein.

The framework may be treated prior to inoculation of the amnion derived adherent cells provided herein in order to enhance cell attachment. For example, prior to inoculation with the cells of the invention, nylon matrices could be treated with 0.1 molar acetic acid and incubated in polylysine, PBS, and/or collagen to coat the nylon. Polystyrene can be similarly treated using sulfuric acid.

In addition, the external surfaces of the three-dimensional framework may be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, or plant gums.

In some embodiments, the matrix comprises or is treated with materials that render the matrix non-thrombogenic, e.g., natural materials such as basement membrane proteins such as laminin and Type IV collagen, and synthetic materials such as ePTFE or segmented polyurethaneurea silicones, such as PURSPAN (The Polymer Technology Group, Inc., Berkeley, Calif.). Such materials can be further treated to render the scaffold non-thrombogenic, e.g., with heparin, and treatments that alter the surface charge of the material, such as plasma coating.

The therapeutic cell compositions comprising amnion derived adherent cells can also be provided in the form of a matrix-cell complex. Matrices can include biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. In certain embodiments, the cells adhere to the matrix. In other embodiments, the cells are entrapped or contained within matrix spaces. Most preferred are those matrix-cell complexes in which the cells grow in close association with the matrix and when used therapeutically, stimulate and support ingrowth of a recipient's cells. The matrix-cell compositions can be introduced into an individual's body in any way known in the art, including but not limited to implantation, injection, surgical attachment, transplantation with other tissue, injection, and the like. In some embodiments, the matrices form in vivo, or in situ. For example, in situ polymerizable gels can be used in accordance with the invention. Examples of such gels are known in the art.

In some embodiments, the cells provided herein are seeded onto such three-dimensional matrices, such as scaffolds and implanted in vivo, where the seeded cells may proliferate on or in the framework or help establish replacement tissue in vivo with or without cooperation of other cells. Growth of the amnion derived adherent cells or co-cultures thereof on the three-dimensional framework preferably results in the formation of a three-dimensional tissue, or foundation thereof, which can be utilized in vivo, for example for repair of damaged or diseased tissue. For example, the three-dimensional scaffolds can be used to form tubular structures, for example for use in repair of blood vessels; or aspects of the circulatory system or coronary structures. In accordance with one aspect of the invention, amnion derived adherent cells, or co-cultures thereof, are inoculated, or seeded on a three-dimensional framework or matrix, such as a scaffold, a foam or hydrogel. The framework may be configured into various shapes such as generally flat, generally cylindrical or tubular, or can be completely free-form as may be required or desired for the corrective structure under consideration. In some embodiments, the amnion derived adherent cells grow on the three dimensional structure, while in other embodiments, the cells only survive, or even die, but stimulate or promote ingrowth of new tissue or vascularization in a recipient.

The cells of the invention can be grown freely in culture, removed from the culture and inoculated onto a three-dimensional framework. Inoculation of the three-dimensional framework with a concentration of cells, e.g., approximately 10⁶ to 5×10⁷ cells per milliliter, preferably results in the establishment of the three-dimensional support in relatively shorter periods of time. Moreover in some application it may be preferably to use a greater or lesser number of cells depending on the result desired.

In a specific embodiment, the matrix can be cut into a strip (e.g., rectangular in shape) of which the width is approximately equal to the inner circumference of a tubular organ into which it will ultimately be inserted. The amnion derived adherent cells can be inoculated onto the scaffold and incubated by floating or suspending in liquid media. At the appropriate stage of confluence, the scaffold can be rolled up into a tube by joining the long edges together. The seam can then be closed by suturing the two edges together using fibers of a suitable material of an appropriate diameter. In order to prevent cells from occluding the lumen, one of the open ends of the tubular framework can be affixed to a nozzle. Liquid media can be forced through the nozzle from a source chamber connected to the incubation chamber to create a current through the interior of the tubular framework. The other open end can be affixed to an outflow aperture which leads into a collection chamber from which the media can be recirculated through the source chamber. The tube can be detached from the nozzle and outflow aperture when incubation is complete. See, e.g., International Application No. WO 94/25584.

In general, two three-dimensional frameworks can be combined into a tube in accordance with the invention using any of the following methods. Two or more flat frameworks can be laid atop another and sutured together. The resulting two-layer sheet can then be rolled up, and, as described above, joined together and secured. In certain embodiments, one tubular scaffold that is to serve as the inner layer can be inoculated with amnion derived adherent cells and incubated. A second scaffold can be grown as a flat strip with width slightly larger than the outer circumference of the tubular framework. After appropriate growth is attained, the flat framework is wrapped around the outside of the tubular scaffold followed by closure of the seam of the two edges of the flat framework and securing the flat framework to the inner tube. In another embodiment, two or more tubular meshes of slightly differing diameters can be grown separately. The framework with the smaller diameter can be inserted inside the larger one and secured. For each of these methods, more layers can be added by reapplying the method to the double-layered tube. The scaffolds can be combined at any stage of growth of the amnion derived adherent cells, and incubation of the combined scaffolds can be continued when desirable.

In conjunction with the above, the cells and therapeutic compositions provided herein can be used in conjunction with implantable devices. For example the amnion derived adherent cells can be coadministered with, for example, stents, artificial valves, ventricular assist devices, Guglielmi detachable coils and the like. As the devices may constitute the dominant therapy provided to an individual in need of such therapy, the cells and the like may be used as supportive or secondary therapy to assist in, stimulate, or promote proper healing in the area of the implanted device. The cells and therapeutic compositions of the invention may also be used to pretreat certain implantable devices, to minimize problems when they are used in vivo. Such pretreated devices, including coated devices, may be better tolerated by patients receiving them, with decrease risk of local or systemic infection, or for example, restenosis or further occlusion of blood vessels.

5.8.3 Media Conditioned by Amnion Derived Adherent Cells

Further provided herein is medium that has been conditioned by amnion derived adherent cells, that is, medium comprising one or more biomolecules secreted or excreted by the adherent cells. In various embodiments, the conditioned medium comprises medium in which the cells have grown for at least 1, 2, 3, 4, 5, 6, 7, 8, 9, 10, 11, 12, 13, 14 or more days, or for at least 1, 2, 3, 4, 5, 6, 7, 8, 9, 10, 11, 12, 13, 14, 15, 16, 17, 18, 19, or 20 population doublings, or more. In other embodiments, the conditioned medium comprises medium in which amnion derived adherent cells have grown to at least 30%, 40%, 50%, 60%, 70%, 80%, 90% confluence, or up to 100% confluence. Such conditioned medium can be used to support the culture of a population of cells, e.g., stem cells, e.g., placental stem cells, embryonic stem cells, embryonic germ cells, adult stem cells, or the like. In another embodiment, the conditioned medium comprises medium in which amnion derived adherent cells, and cells that are not amnion derived adherent cells, have been cultured together.

The conditioned medium can comprise the adherent cells provided herein. Thus, provided herein is a cell culture comprising amnion derived adherent cells. In a specific embodiment, the conditioned medium comprises a plurality, e.g., a population, of amnion derived adherent cells.

The conditioned medium can be collected from the cell culture and filtered and/or sterilized using methods known in the art, e.g., the conditioned medium can be sterilized to neutralize the activity of any potential contaminants of filtered through a small pore filter (e.g., a 0.22 μM filter) to remove contaminants. In some embodiments, the conditioned medium can be used immediately after collection and sterilization/filtration in a method of treatment provided herein. In other embodiments, the conditioned medium can be frozen and stored for subsequent use in a method of treatment provided herein.

5.9 Preservation of Amnion Derived Adherent Cells

Amnion derived adherent cells can be preserved, that is, placed under conditions that allow for long-term storage, or conditions that inhibit cell death by, e.g., apoptosis or necrosis, e.g., during collection or prior to production of the compositions described herein, e.g., using the methods described herein.

Amnion derived adherent cells can be preserved using, e.g., a composition comprising an apoptosis inhibitor, necrosis inhibitor and/or an oxygen-carrying perfluorocarbon, as described in U.S. Application Publication No. 2007/0190042, the disclosure of which is hereby incorporated by reference in its entirety. In one embodiment, a method of preserving such cells, or a population of such cells, comprises contacting said cells or population of cells with a cell collection composition comprising an inhibitor of apoptosis and an oxygen-carrying perfluorocarbon, wherein said inhibitor of apoptosis is present in an amount and for a time sufficient to reduce or prevent apoptosis in the population of cells, as compared to a population of cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of amnion derived adherent cells. In another embodiment, said cell collection composition comprises said inhibitor of apoptosis and said oxygen-carrying perfluorocarbon in separate phases. In another embodiment, said cell collection composition comprises said inhibitor of apoptosis and said oxygen-carrying perfluorocarbon in an emulsion. In another embodiment, the cell collection composition additionally comprises an emulsifier, e.g., lecithin. In another embodiment, said apoptosis inhibitor and said perfluorocarbon are between about 0° C. and about 25° C. at the time of contacting the cells. In another more specific embodiment, said apoptosis inhibitor and said perfluorocarbon are between about 2° C. and 10° C., or between about 2° C. and about 5° C., at the time of contacting the cells. In another more specific embodiment, said contacting is performed during transport of said population of cells. In another more specific embodiment, said contacting is performed during freezing and thawing of said population of cells.

Populations of amnion derived adherent cells can be preserved, e.g., by a method comprising contacting a population of said cells with an inhibitor of apoptosis and an organ-preserving compound, wherein said inhibitor of apoptosis is present in an amount and for a time sufficient to reduce or prevent apoptosis in the population of cells, as compared to a population of cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267. In another embodiment, said organ-preserving compound is hydroxyethyl starch, lactobionic acid, raffinose, or a combination thereof. In another embodiment, the cell collection composition additionally comprises an oxygen-carrying perfluorocarbon, either in two phases or as an emulsion.

In another embodiment of the method, amnion derived adherent cells are contacted with a cell collection composition comprising an apoptosis inhibitor and oxygen-carrying perfluorocarbon, organ-preserving compound, or combination thereof, during a process of tissue disruption, e.g., enzymatic digestion of amnion tissue. In another embodiment, amnion derived adherent cells are contacted with said cell collection compound after collection by tissue disruption, e.g., enzymatic digestion of amnion tissue.

Typically, during collection of amnion derived adherent cells, enrichment and isolation, it is preferable to minimize or eliminate cell stress due to hypoxia and mechanical stress. In another embodiment of the method, therefore, an amnion derived adherent cell, or population of cells comprising the amnion derived adherent cells, is exposed to a hypoxic condition during collection, enrichment or isolation for less than six hours during said preservation, wherein a hypoxic condition is a concentration of oxygen that is, e.g., less than normal atmospheric oxygen concentration; less than normal blood oxygen concentration; or the like. In a more specific embodiment, said cells or population of said cells is exposed to said hypoxic condition for less than two hours during said preservation. In another more specific embodiment, said cells or population of said cells is exposed to said hypoxic condition for less than one hour, or less than thirty minutes, or is not exposed to a hypoxic condition, during collection, enrichment or isolation. In another specific embodiment, said population of cells is not exposed to shear stress during collection, enrichment or isolation.

Amnion derived adherent cells can be cryopreserved, in general or by the specific methods disclosed herein, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., cell freezing medium identified by SigmaAldrich catalog numbers C2695, C2639 (Cell Freezing Medium-Serum-free 1×, not containing DMSO) or C6039 (Cell Freezing Medium-Glycoerol 1× containing Minimum Essential Medium, glycerol, calf serum and bovine serum), Lonza PROFREEZE™ 2× Medium, methylcellulose, dextran, human serum albumin, fetal bovine serum, fetal calf serum, or Plasmalyte. Cryopreservation medium preferably comprises DMSO (dimethylsulfoxide) or glycerol, at a concentration of, e.g., about 1% to about 20%, e.g., about 5% to 10% (v/v), optionally including fetal bovine serum or human serum. Cryopreservation medium may comprise additional agents, for example, methylcellulose with or without glycerol. Isolated amnion derived adherent cells are preferably cooled at about 1° C./min during cryopreservation. A preferred cryopreservation temperature is about −80° C. to about −180° C., preferably about −125° C. to about −140° C. Cryopreserved cells can be transferred to vapor phase of liquid nitrogen prior to thawing for use. In some embodiments, for example, once the ampoules have reached about −80° C., they are transferred to a liquid nitrogen storage area. Cryopreservation can also be done using a controlled-rate freezer. Cryopreserved cells preferably are thawed at a temperature of about 25° C. to about 40° C., preferably to a temperature of about 37° C.

5.10 Modified Amnion Derived Adherent Cells

5.10.1 Genetically Modified Amnion Derived Adherent Cells

In another aspect, the amnion derived adherent cells described herein can be genetically modified, e.g., to produce a nucleic acid or polypeptide of interest, or to produce a differentiated cell, e.g., an osteogenic cell, myocytic cell, pericytic cell, or angiogenic cell, that produces a nucleic acid or polypeptide of interest. Genetic modification can be accomplished, e.g., using virus-based vectors including, but not limited to, non-integrating replicating vectors, e.g., papilloma virus vectors, SV40 vectors, adenoviral vectors; integrating viral vectors, e.g., retrovirus vector or adeno-associated viral vectors; or replication-defective viral vectors. Other methods of introducing DNA into cells include the use of liposomes, electroporation, a particle gun, direct DNA injection, or the like.

The adherent cells provided herein can be, e.g., transformed or transfected with DNA controlled by or in operative association with, one or more appropriate expression control elements, for example, promoter or enhancer sequences, transcription terminators, polyadenylation sites, internal ribosomal entry sites. Preferably, such a DNA incorporates a selectable marker. Following the introduction of the foreign DNA, engineered adherent cells can be, e.g., grown in enriched media and then switched to selective media. In one embodiment, the DNA used to engineer an amnion derived adherent cell comprises a nucleotide sequence encoding a polypeptide of interest, e.g., a cytokine, growth factor, differentiation agent, or therapeutic polypeptide.

The DNA used to engineer the adherent cell can comprise any promoter known in the art to drive expression of a nucleotide sequence in mammalian cells, e.g., human cells. For example, promoters include, but are not limited to, CMV promoter/enhancer, SV40 promoter, papillomavirus promoter, Epstein-Barr virus promoter, elastin gene promoter, and the like. In a specific embodiment, the promoter is regulatable so that the nucleotide sequence is expressed only when desired. Promoters can be either inducible (e.g., those associated with metallothionein and heat shock proteins) or constitutive.

In another specific embodiment, the promoter is tissue-specific or exhibits tissue specificity. Examples of such promoters include but are not limited to myosin light chain-2 gene control region (Shani, 1985, Nature 314:283) (skeletal muscle).

The amnion derived adherent cells disclosed herein may be engineered or otherwise selected to “knock out” or “knock down” expression of one or more genes in such cells. The expression of a gene native to a cell can be diminished by, for example, inhibition of expression by inactivating the gene completely by, e.g., homologous recombination. In one embodiment, for example, an exon encoding an important region of the protein, or an exon 5′ to that region, is interrupted by a positive selectable marker, e.g., neo, preventing the production of normal mRNA from the target gene and resulting in inactivation of the gene. A gene may also be inactivated by creating a deletion in part of a gene or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084). Antisense, morpholinos, DNAzymes, small interfering RNA, short hairpin RNA, and ribozyme molecules that inhibit expression of the target gene can also be used to reduce the level of target gene activity in the adherent cells. For example, antisense RNA molecules which inhibit the expression of major histocompatibility gene complexes (HLA) have been shown to be most versatile with respect to immune responses. Triple helix molecules can be utilized in reducing the level of target gene activity. See, e.g., L. G. Davis et al. (eds), 1994, BASIC METHODS 1N MOLECULAR BIOLOGY, 2nd ed., Appleton & Lange, Norwalk, Conn., which is incorporated herein by reference.

In a specific embodiment, the amnion derived adherent cells disclosed herein can be genetically modified with a nucleic acid molecule comprising a nucleotide sequence encoding a polypeptide of interest, wherein expression of the polypeptide of interest is controllable by an exogenous factor, e.g., polypeptide, small organic molecule, or the like. The polypeptide of interest can be a therapeutic polypeptide. In a more specific embodiment, the polypeptide of interest is IL-12 or interleukin-1 receptor antagonist (IL-1Ra). In another more specific embodiment, the polypeptide of interest is a fusion of interleukin-1 receptor antagonist and dihydrofolate reductase (DHFR), and the exogenous factor is an antifolate, e.g., methotrexate. Such a construct is useful in the engineering of amnion derived adherent cells that express IL-1Ra, or a fusion of IL-1Ra and DHFR, upon contact with methotrexate. Such a construct can be used, e.g., in the treatment of rheumatoid arthritis. In this embodiment, the fusion of IL-1Ra and DHFR is translationally upregulated upon exposure to an antifolate such as methotrexate. Therefore, in another specific embodiment, the nucleic acid used to genetically engineer an amnion derived adherent cell can comprise nucleotide sequences encoding a first polypeptide and a second polypeptide, wherein said first and second polypeptides are expressed as a fusion protein that is translationally upregulated in the presence of an exogenous factor. The polypeptide can be expressed transiently or long-term (e.g., over the course of weeks or months). Such a nucleic acid molecule can additionally comprise a nucleotide sequence encoding a polypeptide that allows for positive selection of engineered cells, or allows for visualization of the engineered cells. In another more specific embodiment, the nucleotide sequence encodes a polypeptide that is, e.g., fluorescent under appropriate visualization conditions, e.g., luciferase (Luc). In a more specific embodiment, such a nucleic acid molecule can comprise IL-1Ra-DHFR-IRES-Luc, where IL-1Ra is interleukin-1 receptor antagonist, IRES is an internal ribosomal entry site, and DHFR is dihydrofolate reductase.

5.10.2 Immortalized Amnion Derived Adherent Cell Lines

Mammalian amnion derived adherent cells can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, E1a adenovirus or E7 protein of human papillomavirus. In another embodiment, amnion derived adherent cells can be immortalized using cre-lox recombination, as exemplified for a human pancreatic β-cell line by Narushima, M., et al (Nature Biotechnology, 2005, 23(10:1274-1282).

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells, in one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV*-1}, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the present methods. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized amnion derived adherent cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized adherent cell line prepared as described above. In general, such clonal cell lines may be isolated using standard techniques, such as by limit dilution or using cloning rings, and expanded. Clonal cell lines may generally be fed and passaged as described above.

Conditionally-immortalized human amnion derived adherent cells lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

5.11 Dosages and Routes of Administration

Administration of amnion derived adherent cells (AMDACs) to an individual in need thereof can be by any medically-acceptable route relevant for the disease, disorder or condition associated with CNS injury to be treated. In a specific embodiment of the methods of treatment described above, said AMDACs are administered by bolus injection. In another specific embodiment, said isolated AMDACs are administered intravenously, e.g., by intravenous infusion. In a specific embodiment, said intravenous infusion is intravenous infusion over about 1 to about 8 hours. In another specific embodiment, said isolated AMDACs are administered locally, e.g., at a particular site in the body of the individual that is affected by the disease, disorder or condition associated with CNS injury. In another specific embodiment, said isolated AMDACs are administered intracranially. In another specific embodiment, said isolated AMDACs are administered intramuscularly. In another specific embodiment, said isolated AMDACs are administered intraperitoneally. In another specific embodiment, said isolated AMDACs are administered intra-arterially. In another specific embodiment of the method of treatment, said isolated AMDACs are administered intramuscularly, intradermally, or subcutaneously. In another specific embodiment, said isolated AMDACs are administered intravenously. In another specific embodiment, said isolated AMDACs are administered intraventricularly. In another specific embodiment, said isolated AMDACs are administered intrasternally. In another specific embodiment, said isolated AMDACs are administered intrasynovially. In another specific embodiment, said isolated AMDACs are administered intraocularly. In another specific embodiment, said isolated AMDACs are administered intravitreally. In another specific embodiment, said isolated AMDACs are administered intracerebrally. In another specific embodiment, said isolated AMDACs are administered intracerebroventricularly. In another specific embodiment, said isolated AMDACs are administered intrathecally. In another specific embodiment, said isolated AMDACs are administered by intraosseous infusion. In another specific embodiment, said isolated AMDACs are administered intravesically. In another specific embodiment, said isolated AMDACs are administered transdermally. In another specific embodiment, said isolated AMDACs are administered intracisternally. In another specific embodiment, said isolated AMDACs are administered epidurally.

In another specific embodiment of the methods of treatment described above, said AMDACs are administered once to said individual. In another specific embodiment, said isolated AMDACs are administered to said individual in two or more separate administrations. In another specific embodiment, said administering comprises administering between about 1×10⁴ and 1×10⁵ isolated AMDACs, e.g., AMDACs per kilogram of said individual. In another specific embodiment, said administering comprises administering between about 1×10⁵ and 1×10⁶ isolated AMDACs per kilogram of said individual. In another specific embodiment, said administering comprises administering between about 1×10⁶ and 1×10⁷ isolated AMDACs per kilogram of said individual. In another specific embodiment, said administering comprises administering between about 1×10⁷ and 1×10⁸ isolated AMDACs per kilogram of said individual. In another specific embodiment, said administering comprises administering between about 1×10⁸ and 1×10⁹ isolated AMDACs per kilogram of said individual. In another specific embodiment, said administering comprises administering between about 1×10⁹ and 1×10¹⁰ isolated AMDACs per kilogram of said individual. In another specific embodiment, said administering comprises administering between about 1×10¹⁰ and 1×10¹¹ isolated AMDACs per kilogram of said individual. In other specific embodiments, said administering comprises administering between about 1×10⁶ and about 2×10⁶ isolated AMDACs per kilogram of said individual; between about 2×10⁶ and about 3×10⁶ isolated AMDACs per kilogram of said individual; between about 3×10⁶ and about 4×10⁶ isolated AMDACs per kilogram of said individual; between about 4×10⁶ and about 5×10⁶ isolated AMDACs per kilogram of said individual; between about 5×10⁶ and about 6×10⁶ isolated AMDACs per kilogram of said individual; between about 6×10⁶ and about 7×10⁶ isolated AMDACs per kilogram of said individual; between about 7×10⁶ and about 8×10⁶ isolated AMDACs per kilogram of said individual; between about 8×10⁶ and about 9×10⁶ isolated AMDACs per kilogram of said individual; or between about 9×10⁶ and about 1×10⁷ isolated AMDACs per kilogram of said individual. In another specific embodiment, said administering comprises administering between about 1×10⁷ and about 2×10⁷ isolated AMDACs per kilogram of said individual to said individual. In another specific embodiment, said administering comprises administering between about 1.3×10⁷ and about 1.5×10⁷ isolated AMDACs per kilogram of said individual to said individual. In another specific embodiment, said administering comprises administering up to about 3×10⁷ isolated AMDACs per kilogram of said individual to said individual. In a specific embodiment, said administering comprises administering between about 5×10⁶ and about 2×10⁷ isolated AMDACs to said individual. In another specific embodiment, said administering comprises administering about 150×10⁶ isolated AMDACs in about 20 milliliters of solution to said individual.

In another specific embodiment of the methods of treatment described above, isolated AMDACs are administered to an individual as a single unit dose. In specific embodiments, a single unit dose of AMDACs can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more AMDACs.

In a specific embodiment, said administering comprises administering between about 5×10⁶ and about 2×10⁷ isolated AMDACs to said individual, wherein said cells are contained in a solution comprising 10% dextran, e.g., dextran-40, 5% human serum albumin, and optionally an immunosuppressant. In another specific embodiment, said administering comprises administering between about 5×10⁷ and 3×10⁹ isolated AMDACs intravenously. In more specific embodiments, said administering comprises administering about 9×10⁸ isolated AMDACs or about 1.8×10⁹ isolated AMDACs intravenously. In another specific embodiment, said administering comprises administering between about 5×10⁷ and 1×10⁸ isolated AMDACs intracranially. In a more specific embodiment, said administering comprises administering about 9×10⁷ isolated AMDACs intracranially.

Administration of medium conditioned by AMDACs to an individual in need thereof can be by any medically-acceptable route relevant for the disease, disorder or condition associated with CNS injury to be treated including, but not limited to, bolus injection, intravenously (e.g., by intravenous infusion), locally (e.g., at a particular site in the body of the individual that is affected by the disease, disorder or condition associated with CNS injury), intracranially, intramuscularly, intraperitoneally, intra-arterially, intramuscularly, intradermally, subcutaneously, intraventricularly, intrasynovially, intraocularly, intravitreally, intracerebrally, intracerebroventricularly, intrathecally, by intraosseous infusion, intravesically, transdermally, intracistemally, or epidurally. In a specific embodiment, the medium conditioned by AMDACs is administered by continuous infusion. In another specific embodiment, the medium conditioned by AMDACs is administered as a single dose.

In some embodiments, administration of medium conditioned by AMDACs to an individual in need thereof comprises administering about 0.01 to about 0.02 ml of medium conditioned by AMDACs per 100 grams of body weight, about 0.01 to about 0.05 ml of medium conditioned by AMDACs per 100 grams of body weight, about 0.01 to about 0.1 ml of medium conditioned by AMDACs per 100 grams of body weight, about 0.01 to about 0.15 ml of medium conditioned by AMDACs per 100 grams of body weight, about 0.01 to about 0.2 ml of medium conditioned by AMDACs per 100 grams of body weight, about 0.01 to about 0.25 ml of medium conditioned by AMDACs per 100 grams of body weight, about 0.01 to about 0.3 ml of medium conditioned by AMDACs per 100 grams of body weight, about 0.01 to about 0.35 ml of medium conditioned by AMDACs per 100 grams of body weight, about 0.01 to about 0.4 ml of medium conditioned by AMDACs per 100 grams of body weight, about 0.01 to about 0.45 ml of medium conditioned by AMDACs per 100 grams of body weight, or about 0.01 to about 0.5 ml of medium conditioned by AMDACs per 100 grams of body weight.

5.12 Differentiation of Amnion Derived Adherent Cells

The amnion derived adherent cells provided herein can be differentiated. In one embodiment, the cell has been differentiated sufficiently for said cell to exhibit at least one characteristic of an endothelial cell, a myogenic cell, or a pericytic cell, e.g., by contacting the cell with vascular endothelial growth factor (VEGF), or as described in Sections 5.11.2, 6.3.3, or 6.3.4, below. In more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, CD9⁺, CD54⁺, CD105⁺, CD200⁺, and VE-cadherin⁻. In other more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, and VEGFR1/Flt-1⁺.

Myogenic (cardiogenic) differentiation of the amnion derived adherent cells provided herein can be accomplished, for example, by placing the cells in cell culture conditions that induce differentiation into cardiomyocytes. A preferred cardiomyocytic medium comprises DMEM/20% CBS supplemented with retinoic acid, 1 μM; basic fibroblast growth factor, 10 ng/mL; and transforming growth factor beta-1, 2 ng/mL; and epidermal growth factor, 100 ng/mL. KnockOut Serum Replacement (Invitrogen, Carlsbad, Calif.) may be used in lieu of CBS. Alternatively, the amnion derived adherent cells are cultured in DMEM/20% CBS supplemented with 1 to 100, e.g., 50 ng/mL Cardiotropin-1 for 24 hours. In another embodiment, amnion derived adherent cells can be cultured 10-14 days in protein-free medium for 5-7 days, then stimulated with human myocardium extract, e.g., produced by homogenizing human myocardium in 1% HEPES buffer supplemented with 1% cord blood serum.

Differentiation can be confirmed by demonstration of cardiac actin gene expression, e.g., by RT/PCR, or by visible beating of the cell. An adherent cell is considered to have differentiated into a cardiac cell when the cell displays one or more of these characteristics.

5.12.1 Differentiation into Neurogenic Cells

Amnion derived angiogenic cells, when cultured under neurogenic conditions, differentiate into cells displaying neural morphology and neural markers. For example, AMDACs, e.g., AMDACs expanded for 4 days in DMEM/F12 medium containing 15% v/v FBS, with basic fibroblast growth factor (bFGF), e.g., at about 20 ng/ml, epidermal growth factor (EGF), e.g., at about 20 ng/ml, e.g., for four days, followed by culture for four days in induction medium comprising DMEM/F12, serum free, containing 200 mM butylated hydroxyanisole, 10 nM potassium chloride, 5 mgs/mL insulin, 10 nM forskolin, 4 nM valproic acid, and 2 nM hydrocortisone. Under these conditions, AMDACs display expression of human nestin, Tuj1 and GFAP, as assessed by antibody staining.

5.12.2 Non-Differentiation into Osteogenic Cells

Amnion derived adherent cells do not show osteogenic differentiation in standard assays for osteogenesis. For example, in one embodiment, lack of osteogenic differentiation by AMDACs can be shown, e.g., by lack of deposition of calcium, as shown by lack of von Kossa staining of AMDACs under osteogenic conditions. For example, AMDACs, e.g., freshly-prepared or cryopreserved AMDACs, can be suspended in growth medium, e.g., at about 5000 cells/cm² in 24-well plates and 6-well plates in growth medium and incubated overnight, then cultured for 14-35 days, e.g., 28, days in osteogenic medium. In certain embodiments, osteogenic medium comprises DMEM-low glucose, 10% v/v fetal bovine serum (FBS), 10 mM beta glycerophosphate, 100 nM dexamethasone, and 100 μM ascorbic acid phosphate salt supplemented with transforming growth factor-beta1 (TGF-β1), e.g., at 1-100 ng/mL, e.g., 20 ng/mL, and human recombinant bone morphogenetic protein-2 (BMP-2) at, e.g., 1-100 ng/mL, e.g., 40 ng/mL. Cells are then stained using von Kossa stain using standard protocols; development of black silver deposits indicates the presence of mineralization. In the case of AMDACs, cultures should be substantially, e.g., completely, free of deposits, e.g., as compared to bone marrow-mesenchymal stem cells, indicating that the AMDACs do not produce calcium deposits, and therefore do not differentiate down an osteogenic pathway.

5.12.3 Non-Differentiation into Chondrogenic Cells

Amnion derived adherent cells similarly do not show chondrogenic differentiation in standard assays for chondrogenesis. For example, in one embodiment, lack of chondrogenic differentiation by AMDACs can be shown, e.g., by lack of development by AMDACs of cell pellets in a chondrogenesis assay in which chondrogenic cells for cell pellets. For example, AMDACs, e.g., freshly prepared or cryopreserved, e.g., 2.5×10⁵ cells, can be placed in 15 mL conical tubes and centrifuged at 200×g for 5 minutes at room temperature to form a spherical cell pellet. The collected cells are then cultured in chondrogenic induction medium, e.g., Lonza Chondrocyte Medium containing TGF beta-3 (e.g., at about 10 ng/mL), recombinant human growth/differentiation factor-5 (rhGDF-5) (e.g., at about 500 ng/mL), or a combination of TGF beta-3 (10 nanogram/milliliter), and rhGDF-5 (e.g., at about 500 ng/mL) for three weeks. At the end of three weeks, the cells are stained with Alcian blue, which stains for mucopolysaccharides and glycosaminoglycans that are produced by chondrogenic cells. Typically, while BM-MSCs or chondrocytes will, when cultured under these conditions, develop cell pellets that stain positively for Alcian blue, AMDACs neither form pellets nor stain with Alcian blue.

### 6. EXAMPLES

### 6.1 Example 1

### Isolation and Expansion of Adherent Cells from Amniotic Membrane

This Example demonstrates the isolation and expansion of amnion derived adherent cells.

6.1.1 Isolation

Amnion derived adherent cells were isolated from amniotic membrane as follows. Amnion/chorion were cut from the placenta, and amnion was manually separated from the chorion. The amnion was rinsed with sterile PBS to remove residual blood, blood clots and other material. Sterile gauze was used to remove additional blood, blood clots or other material that was not removed by rinsing, and the amnion was rinsed again with PBS. Excess PBS was removed from the membrane, and the amnion was cut with a scalpel into 2″ by 2″ segments. For epithelial cell release, a processing vessel was set up by connecting a sterile jacketed glass processing vessel to a circulating 37° C. water bath using tubing and connectors, and set on a stir plate. Trypsin (0.25%, 300 mL) was warmed to 37° C. in the processing vessel; the amnion segments were added, and the amnion/trypsin suspension was agitated, e.g., at 100 RPM-150 RPM at 37° C. for 15 minutes. A sterile screening system was assembled by placing a sterile receptacle on a sterile field next to the processing vessel and inserting a sterile 75 μm to 125 μm screen into the receptacle (Millipore, Billerica, Mass.). After agitating the amnion segments for 15 minutes, the contents of the processing vessel were transferred to the screen, and the amnion segments were transferred, e.g., using sterile tweezers back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel, and the trypsin solution containing the epithelial cells was discarded. The amnion segments were agitated in PBS/5% FBS (1:1 ratio of amnion to PBS/5% FBS solution by volume) at 37° C. for approximately 2-5 minutes to neutralize the trypsin. A fresh sterile screen system was assembled. After neutralizing the trypsin, the contents of the processing vessel were transferred to the new screen, and the amnion segments were transferred back into the processing vessel. Room temperature, sterile PBS (400 mL) was added to the processing vessel, and the contents of the processing vessel were agitated for approximately 2-5 minutes. The screen was rinsed with approximately 100-150 mL of PBS. After agitation, the contents of the processing vessel were transferred to the screen; the processing flask was rinsed with PBS, and the PBS solution was discarded. The processing vessel was then filled with 300 mL of pre-warmed DMEM, and the amnion segments were transferred into the DMEM solution.

For release of the amnion derived adherent cells, the treated amniotic membrane was further treated with collagenase as follows. A sterile collagenase stock solution (500 U/mL) was prepared by dissolving the appropriate amount of collagenase powder (varied with the activity of the collagenase lot received from the supplier) in DMEM. The solution was filtered through a 0.22 μm filter and dispensed into individual sterile containers. CaCl₂ solution (0.5 mL, 600 mM) was added to each 100 mL dose, and the doses were frozen. Collagenase (100 mL) was added to the amnion segments in the processing vessel, and the processing vessel was agitated for 30-50 minutes, or until amnion digestion was complete by visual inspection. After amnion digestion was complete, 100 mL of pre-warmed sterile PBS/5% FBS was added to the processing vessel, and the processing vessel was agitated for an additional 2-3 minutes. Following agitation, the contents of the flask were transferred to a sterile 60 μm screen, and the liquid was collected by vacuum filtration. The processing vessel was rinsed with 400 mL of PBS, and the PBS solution was sterile-filtered. The filtered cell suspension was then centrifuged at 300×g for 15 minutes at 20° C., and the cell pellets were resuspended in pre-warmed PBS/2% FBS (approximately 10 mL total).

6.1.2 Establishment

Freshly isolated angiogenic amniotic cells were added to growth medium containing 60% DMEM-LG (Gibco); 40% MCBD-201 (Sigma); 2% FBS (Hyclone Labs), 1× insulin-transferrin-selenium (ITS); 10 ng/mL linoleic acid-bovine serum albumin (LA-BSA); 1 n-dexamethasone (Sigma); 100 μM ascorbic acid 2-phosphate (Sigma); 10 ng/mL epidermal growth factor (R & D Systems); and 10 ng/mL platelet-derived growth factor (PDGF-BB) (R & D Systems) and were plated in a T-Flask at a seeding density of 10,000 cells per cm². The culture device(s) were then incubated at 37° C., 5% CO₂ with >90% humidity. Cellular attachment, growth, and morphology were monitored daily. Non-adherent cells and debris were removed by medium exchange. Medium exchange was performed twice per week. Adherent cells with typical fibroblastoid/spindle shape morphology appeared at several days after initial plating. When confluency reached 40%-70% (at 4-11 days after initial plating), the cells were harvested by trypsinization (0.25% trypsin-EDTA) for 5 minutes at room temperature (37° C.). After neutralization with PBS-5% FBS, the cells were centrifuged at 200-400 g for 5-15 minutes at room temperature, and then were resuspended in growth medium. At this point, an AMDAC line was considered to be successfully established at the initial passage. Initial passage amnion derived adherent cells were, in some cases, cryopreserved or expanded (e.g., grown in culture).

6.1.3 Culture Procedure

Amnion derived adherent cells were cultured in the growth medium described above and seeded at a density of 2000-4000 per cm² in an appropriate tissue culture-treated culture device(s). The culture device(s) were then incubated at 37° C., 5% CO₂ with >90% humidity. During culture, AMDACs would adhere and proliferate. Cellular growth, morphology, and confluency were monitored daily. Medium exchange was performed twice a week to replenish fresh nutrients if the culture extended to 5 days or more. When confluency reached 40%-70% (at 3-7 days after seeding), the cells were harvested by trypsinization (0.05%-0.25% trypsin-EDTA) for 5 minutes at room temperature (37° C.). After neutralization with PBS-5% FBS, the cells were centrifuged at 200-400 g for 5-15 minutes at room temperature, then were resuspended in growth medium.

AMDACs isolated and cultured in this manner typically produced 33530+/−15090 colony-forming units (fibroblast) (CFU-F) out of 1×10⁶ cells plated.

### 6.2 Example 2

### Phenotypic Characterization of Amnion Derived Adherent Cells

6.2.1 Gene and Protein Expression Profiles

This Example describes phenotypic characterization of amnion derived adherent cells, including characteristic cell surface marker, mRNA, and proteomic expression.

Sample preparation: Amnion derived adherent cells were obtained as described in Example 1. The cells at passage 6 were grown to approximately 70% confluence in growth medium as described in Example 1, above, trypsinized, and washed in PBS. NTERA-2 cells (American Type Culture Collection, ATCC Number CRL-1973) were grown in DMEM containing 4.5 g/L glucose, 2 mM glutamine and 10% FBS. Nucleated cell counts were performed to obtain a minimum of 2×10⁶ to 1×10⁷ cells. The cells were then lysed using a Qiagen RNeasy kit (Qiagen, Valencia, Calif.), utilizing a QIAshredder, to obtain the lysates. The RNA isolation was then performed using a Qiagen RNeasy kit. RNA quantity and quality were determined using a Nanodrop ND 1000 spectrophotometer, 25 ng/μL of RNA/reaction. The cDNA reactions were prepared using an Applied Biosystems (Foster City, Calif.) High Capacity cDNA Archive Kit. Real time PCR reactions were performed using TAQMAN® universal PCR master mixes from Applied Biosystems. Reactions were run in standard mode on an Applied Biosystems 7300 Real time PCR system for 40 cycles.

Sample analysis and results: Using the real time PCR methodology and specific TAQMAN® gene expression probes and/or the TAQMAN® human angiogenesis array (Applied Biosystems), cells were characterized for expression of stem cell-related, angiogenic and cardiomyogenic markers. Results were expressed either as the relative expression of a gene of interest in comparison to the pertinent cell controls, or the relative expression (delta Ct) of the gene of interest in comparison to a ubiquitously expressed housekeeping gene (for example, GAPDH, 18S, or GUSB).

Amnion derived adherent cells expressed various, stem-cell related, angiogenic and cardiomyogenic genes and displayed a relative absence of OCT-4 expression in comparison to NTERA-2 cells. Table 6 summarizes the expression of selected angiogenic, cardiomyogenic, and stem cell genes.

TABLE 6
Gene expression profile of amnion derived adherent
cells as determined by RT-PCR.

| AMDAC Marker | Positive | Negative |
|---|---|---|
| ACTA2 | X | |
| ACTC1 | X | |
| ADAMTS1 | X | |
| AMOT | X | |
| ANG | X | |
| ANGPT1 | X | |
| ANGPT2 | X | |
| ANGPT4 | | X |
| ANGPTL1 | X | |
| ANGPTL2 | X | |
| ANGPTL3 | | X |
| ANGPTL4 | X | |
| BAI1 | X | |
| BGLAP | | X |
| c-myc | X | |
| CD31 | | X |
| CD34 | | X |
| CD44 | X | |
| CD140a | X | |
| CD140b | X | |
| CD200 | X | |
| CD202b | X | |
| CD304 | X | |
| CD309 | X | |
| (VEGFR2/KDR) | | |
| CDH5 | | X |
| CEACAM1 | X | |
| CHGA | X | |
| COL15A1 | X | |
| COL18A1 | X | |
| COL4A1 | X | |
| COL4A2 | X | |
| COL4A3 | X | |
| Connexin-43 | X | |
| CSF3 | X | |
| CTGF | X | |
| CXCL10 | | X |
| CXCL12 | X | |
| CXCL2 | X | |
| DLX5 | | X |
| DNMT3B | X | |
| ECGF1 | X | |
| EDG1 | X | |
| EDIL3 | X | |
| ENPP2 | X | |
| EPHB2 | X | |
| F2 | X | |
| FBLN5 | X | |
| FGA | | X |
| FGF1 | X | |
| FGF2 | X | |
| FGF4 | | X |
| FIGF | X | |
| FLT3 | | X |
| FLT4 | X | |
| FN1 | X | |
| FOXC2 | X | |
| Follistatin | X | |
| Galectin-1 | X | |
| GRN | X | |
| HEY1 | X | |
| HGF | X | |
| HLA-G | | X |
| HSPG2 | X | |
| IFNB1 | X | |
| IFNG | | X |
| IL-8 | X | |
| IL-12A | X | |
| ITGA4 | X | |
| ITGAV | X | |
| ITGB3 | X | |
| KLF-4 | X | |
| LECT1 | | X |
| LEP | | X |
| MDK | X | |
| MMP-13 | | X |
| MMP-2 | X | |
| MYOZ2 | X | |
| NANOG | | X |
| NESTIN | | X |
| NRP2 | X | |
| PDGFB | X | |
| PF4 | X | |
| PGK1 | X | |
| PLG | | X |
| POU5F1 (OCT-4) | | X |
| PRL | | X |
| PROK1 | | X |
| PROX1 | X | |
| PTN | X | |
| SEMA3F | X | |
| SERPINB5 | X | |
| SERPINC1 | X | |
| SERPINF1 | X | |
| SOX2 | | X |
| TERT | | X |
| TGFA | X | |
| TGFB1 | X | |
| THBS1 | X | |
| THBS2 | X | |
| TIE1 | X | |
| TIMP2 | X | |
| TIMP3 | X | |
| TNF | X | |
| TNFSF15 | X | |
| TNMD | | X |
| TNNC1 | X | |
| TNNT2 | X | |
| VASH1 | X | |
| VEGF | X | |
| VEGFB | X | |
| VEGFC | X | |
| VEGFR1/FLT-1 | X | |
| XLKD1 | | X |

In a separate experiment, AMDACs were additionally found to express genes for Aryl hydrocarbon receptor nuclear translocator 2 (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB1R), and hepatoycte growth factor receptor (HGFR/c-met).

Flow cytometry was used as a method to quantify phenotypic markers of amnion derived adherent cells to define the identity of the cells. Cell samples were obtained from frozen stocks. Prior to thaw and during reagent preparation, cell vials were maintained on dry ice. Subsequently, samples were thawed rapidly using a 37° C. water bath. Pre-freeze cell counts were used for calculations for initial post-thaw cell number-dependent dilutions. Briefly, cryovials were thawed in a 37° C. water bath for approximately 30 seconds with gentle agitation. Immediately following thawing, approximately 100-200 μL of cold (2 to 8° C.) thawing solution (PBS with 2.5% albumin and 5% Gentran 40) was added to the cryovial and mixed. After gentle mixing, the total volume in the cryovials was transferred into a 15 mL conical tube containing an equal volume of cold (2 to 8° C.) thawing solution. The cells were centrifuged in a conical tube at 400 g for 5 minutes at room temperature before removing the supernatant. The residual volume was measured with a pipette (estimation); the residual volume and cell pellet were resuspended at room temperature in 1% FBS in PBS to achieve a cell concentration of 250×10³ cells/100 μL buffer. For example, 1×10⁶ cells would be resuspended in 400 μL 1% FBS. The cell suspension was placed into pre-labeled 5 mL FACS tubes (Becton Dickinson (BD), Franklin Lakes, N.J.). For each primary antibody isotype, 100 μL of cell suspension was aliquoted into one isotype control tube. Prior to phenotype analysis, the concentrations of all antibodies were optimized to achieve good signal to noise ratios and adequate detection of CD antigens across a potential four-log dynamic range. The volume of each isotype and sample antibody that was used to stain each sample was determined. To standardize the amount of antibody (in μg) in the isotype and sample tubes, the concentration of each antibody was calculated as (1/actual antibody concentration (μg/μL))×(desired final quantity of antibody in μg for 2.5×10⁵ cells)=# μL of antibody added. A master mix of antibodies for both the isotype and the sample was made with the appropriate amount of antibody added to each tube. The cells were stained for 15-20 minutes at room temperature in the dark. After staining, unbound antibody in each sample was removed by centrifugation (400 g×5 minutes) followed by washing using 2 mL 1% FBS PBS (room temperature) before resuspension in 150 μL of room temperature 1% FBS PBS. The samples were then analyzed on Becton Dickinson FACSCalibur, FACSCantoI or BD FACSCantoII flow cytometers prepared for use per manufacturer's instructions. Multi-parametric flow cytometry data sets (side scatter (SSC), forward scatter (FSC) and integrated fluorescence profiles (FL)) were acquired without setting on-the-fly instrument compensation parameters. Compensation parameters were determined after acquisition using the FACSDiva software according to the manufacturer's instructions. These instrument settings were applied to each sample. Fluorophore conjugates used in these studies were Allophycocyanin (APC), AlexaFluor 647 (AF647), Fluorescein isothiocyanate (FITC), Phycoerythrin (PE) and Peridinin chlorophyll protein (PerCP), all from BD Biosciences. Table 7 summarizes the expression of selected cell-surface markers, including angiogenic markers.

TABLE 7
Cell surface marker expression in amnion derived adherent
cells as determined by flow cytometry.

| AMDAC Marker | Positive | Negative |
|---|---|---|
| CD6 | | X |
| CD9 | X | |
| CD10 | X | |
| CD31 | | X |
| CD34 | | X |
| CD44 | X | |
| CD45 | | X |
| CD49b | X | |
| CD49c | X | |
| CD49d | X | |
| CD54 | X | |
| CD68 | X | |
| CD90 | X | |
| CD98 | X | |
| CD105 | X | |
| CD117 | | X |
| CD133 | | X |
| CD143 | | X |
| CD144 | | X |
| (VE-cadherin) | | |
| CD146 | | X |
| CD166 | X | |
| CD184 | | X |
| CD200 | X | |
| CD202b | X | |
| CD271 | | X |
| CD304 | X | |
| CD309 | X | |
| (VEGFR2/KDR) | | |
| CD318 | X | |
| CD349 | X | |
| CytoK | X | |
| HLA-ABC+ B2 | X | |
| Micro+ | | |
| Invariant Chain+ | | X |
| HLA-DR-DP-DQ+ | | |
| PDL-1 | X | |
| VEGFR1/FLT-1 | X | |

In another experiment, AMDAC cells were labeled with anti-human CD49f (Clone GoH3, phycoerythrin-conjugated; BD Pharmingen Part No. 555736), and analyzed by flow cytometry. Approximately 96% of the AMDACs labeled with anti-CD49f (that is, were CD49f⁺).

In other experiments, AMDACs were additionally found by immunolocalization to express CD49a, CD106, CD119, CD130, c-met (hepatocyte growth factor receptor; HGFR), CXC chemokine receptor 1 (CXCR1), PDGFRA, and PDGFRB by immunolocalization. AMDACs were also found, by immunolocalization, to lack expression of CD49e, CD62E, fibroblast growth factor receptor 3 (FGFR3), tumor necrosis factor receptor superfamily member 12A (TNFRSF12A), insulin-like growth factor 1 receptor (IGF-1R), CXCR2, CXCR3, CXCR4, CXCR6, chemokine receptor 1 (CCR1), CCR2, CCR3, CCR4, CCR5, CCR6, CCR7, CCR8, CCR9, epidermal growth factor receptor (EGF-R), insulin receptor (CD220), interleukin receptor 4 (IL4-R; CD124), IL6-R (CD126), TNF-R1a and 1b (CD120a, b), and erbB2/Her2.

6.2.2 Immunohistochemistry (IHC)/Immunofluorochemistry (IFC) for Evaluation of Angiogenic Potency of Amnion Derived Adherent Cells

Amnion derived adherent cells from passage 6 were grown to approximately 70% confluence on 4-well chamber slides and fixed with a 4% formalin solution for 30 minutes each. After fixation, the slides were rinsed with PBS two times for 5 minutes. The slides were then incubated with 10% normal serum from the same host as the secondary antibody, 2× casein, and 0.3% Triton X100 in PBS, for 20 minutes at room temperature in a humid chamber. Excess serum was blotted off and the slides were incubated with primary antibody (goat polyclonal IgG (Santa Cruz; Santa Cruz, Calif.) in a humidified chamber. Time and temperature for incubations were determined by selecting the optimal conditions for the antibody being used. In general, incubation times were 1 to 2 hours at 37° C. or overnight at 4° C. The slides were then rinsed with PBS three times for 5 minutes each and incubated for 20-30 minutes at room temperature in a humid chamber with fluorescent-conjugated anti-immunoglobulin secondary antibody directed against the host of the primary antibody (rabbit anti-goat antibody (Santa Cruz)). Thereafter, the slides were rinsed with PBS three times for 5 minutes each, mounted with a coverslip utilizing DAPI VECTASHIELD® (Vector Labs) mounting solution to counterstain nuclei. Cell staining was visualized utilizing a Nikon fluorescence microscope. All pictures were taken at equal exposure time normalized against the background of the corresponding isotype (goat IgG (Santa Cruz)). Table 8 summarizes the results for the expression of angiogenic proteins by amnion derived adherent cells.

TABLE 8
Angiogenic markers present or absent on
amnion derived adherent cells.

| AMDAC | | |
|---|---|---|
| Marker | Positive | Negative |
| CD31 | | X |
| CD34 | | X |
| (VEGFR2/KDR) | X | |
| Connexin-43 | X | |
| Galectin-1 | X | |
| TEM-7 | X | |

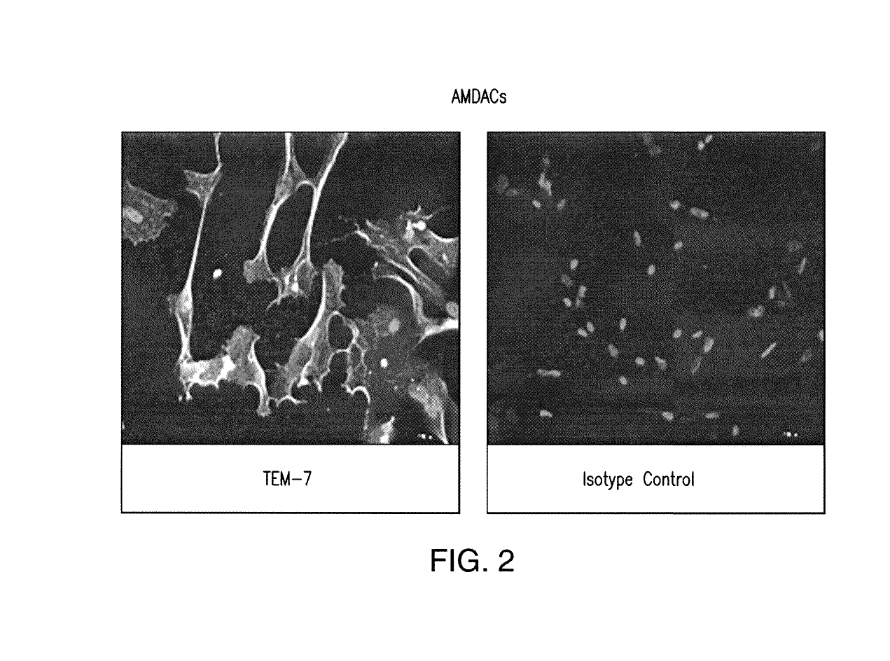

Amnion derived adherent cells expressed the angiogenic marker tumor endothelial marker 7 (TEM-7), one of the proteins shown in Table 8. See FIG. 2.

6.2.3 Membrane Proteomics for Evaluation of Angiogenic Potency of Amnion Derived Adherent Cells

Membrane Protein Purification: Cells at passage 6 were grown to approximately 70% confluence in growth medium, trypsinized, and washed in PBS. The cells were then incubated for 15 minutes with a solution containing protease inhibitor cocktail (P8340, Sigma Aldrich, St. Louis, Mo.) prior to cell lysis. The cells were then lysed by the addition of a 10 mM HCl solution (thus avoiding the use of detergents) and centrifuged for 10 minutes at 400 g to pellet and remove the nuclei. The post-nuclear supernatant was transferred to an ultracentrifugation tube and centrifuged using a WX80 ultracentrifuge with a T-1270 rotor (Thermo Fisher Scientific, Asheville, N.C.) at 100,000 g for 150 minutes generating a membrane protein pellet.

Generation, Immobilization and Digestion of Proteoliposomes: The membrane protein pellet was washed several times using Nanoxis buffer (10 mM Tris, 300 mM NaCl, pH 8). The membrane protein pellet was suspended in 1.5 mL of Nanoxis buffer and then tip-sonicated using a VIBRA-CELL™ VC505 ultrasonic processor (Sonics & Materials, Inc., Newtown, Conn.) for 20 minutes on ice. The size of the proteoliposomes was determined by staining with FM1-43 dye (Invitrogen, Carlsbad, Calif.) and visualization with fluorescence microscopy. The protein concentration of the proteoliposome suspension was determined by a BCA assay (Thermo Scientific). The proteoliposomes were then injected onto an LPI™Flow Cell (Nanoxis AB, Gothenburg, Sweden) using a standard pipette tip and allowed to immobilize for 1 hour. After immobilization, a series of washing steps were carried out and trypsin at 5 μg/mL (Princeton Separations, Adelphi, N.J.) was injected directly onto the LPI™ Flow Cell. The chip was incubated overnight at 37° C. and the tryptic peptides were eluted from the LPI™ chip and then desalted using a Sep-Pak cartridge (Waters Corporation, Milford, Mass.).

LTQ Linear Ion Trap LC/MS/MS Analysis: Each tryptic digest sample was separated on a 0.2 mm×150 mm 3 μm 200 Å MAGIC C18 column (Michrom Bioresources, Inc., Auburn, Calif.) that was interfaced directly to an axial desolvation vacuum-assisted nanocapillary electrospray ionization (ADVANCE) source (Michrom Bioresources, Inc.) using a 180 minute gradient (Buffer A: Water, 0.1% Formic Acid; Buffer B: Acetonitrile, 0.1% Formic Acid). The ADVANCE source achieves a sensitivity that is comparable to traditional nanoESI while operating at a considerably higher flow rate of 3 μL/min. Eluted peptides were analyzed on an LTQ linear ion trap mass spectrometer (Thermo Fisher Scientific, San Jose, Calif.) that employed ten data-dependent MS/MS scans following each full scan mass spectrum. Seven analytical replicate datasets were collected for each biological sample.

Bioinformatics: Seven RAW files corresponding to the 7 analytical replicate datasets that were collected for each cell line were searched as a single search against the IPI Human Database using an implementation of the SEQUEST algorithm on a Sorcerer Solo™ workstation (Sage-N Research, San Jose, Calif.). A peptide mass tolerance of 1.2 amu was specified, oxidation of methionine was specified as a differential modification, and carbamidomethylation was specified as a static modification. Scaffold software implementation of the Trans-Proteomic Pipeline (TPP) was used to sort and parse the membrane proteomic data. Proteins were considered for analysis if they were identified with a peptide probability of 95%, protein probability of 95% and 1 unique peptide. Comparisons between membrane proteomic datasets were made using custom Perl scripts developed in-house.

Results: As shown in Table 9, amnion derived adherent cells expressed various angiogenic and cardiomyogenic markers.

TABLE 9
Cardiomyogenic or angiogenic markers expressed
by amnion derived adherent cells.

| AMDAC Marker | Positive | Negative |
|---|---|---|
| Activin receptor | X | |
| type IIB | | |
| ADAM 17 | X | |
| Alpha-actinin 1 | X | |
| Angiotensinogen | X | |
| Filamin A | X | |
| Macrophage | X | |
| acetylated LDL | | |
| receptor I and II | | |
| Megalin | X | |
| Myosin heavy | X | |
| chain non | | |
| muscle type A | | |
| Myosin-binding | X | |
| protein C cardiac | | |
| type | | |
| Wnt-9 | X | |

6.2.4 Secretome Profiling for Evaluation of Angiogenic Potency of Amnion Derived Adherent Cells

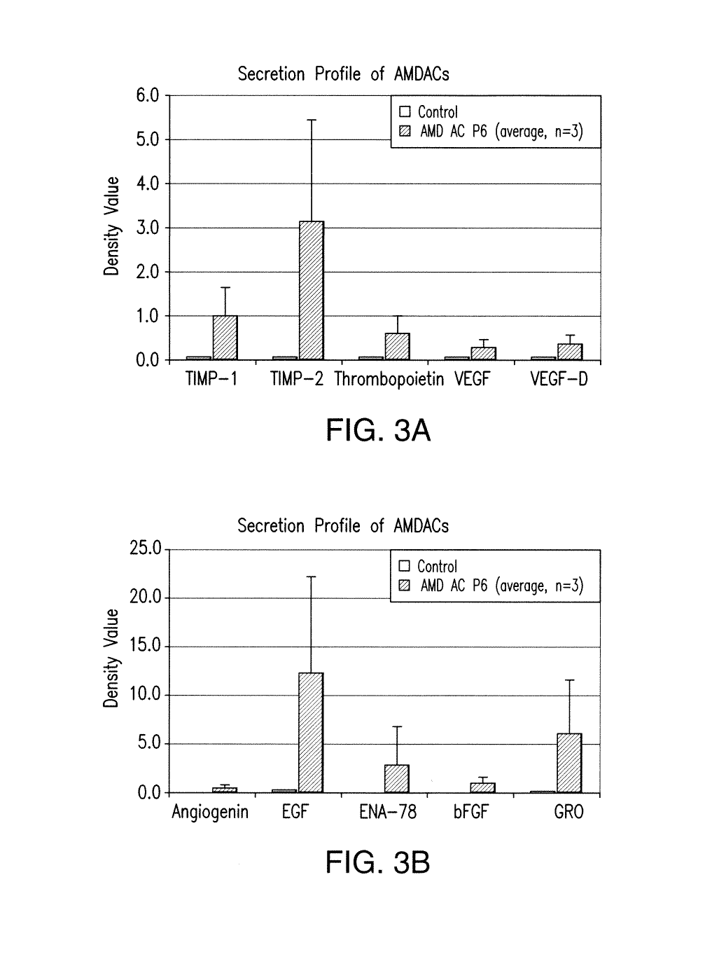

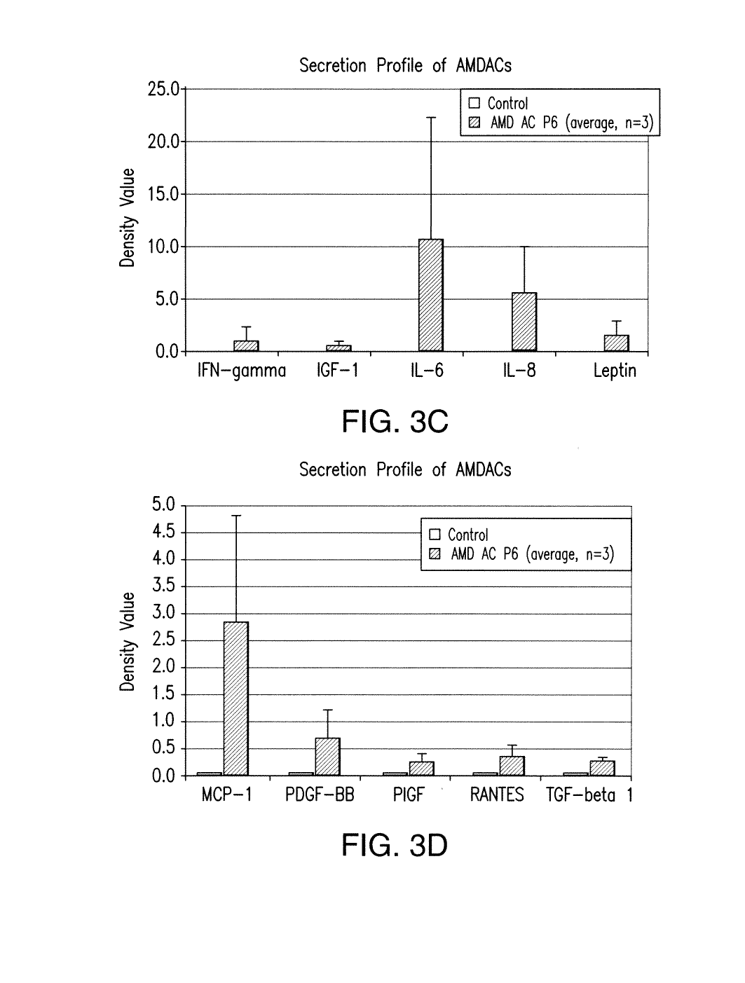

Protein Arrays: Amnion derived adherent cells at passage 6 were plated at equal cell numbers in growth medium and conditioned media were collected after 4 days. Simultaneous qualitative analysis of multiple angiogenic cytokines/growth factors in cell-conditioned media was performed using RayBiotech Angiogenesis Protein Arrays (Norcross, Ga.). In brief, protein arrays were incubated with 2 mL 1× Blocking Buffer (Ray Biotech) at room temperature for 30 minutes (min) to block membranes. Subsequently, the Blocking Buffer was decanted and the membranes were incubated with 1 mL of sample (growth medium conditioned by the respective cells for 4 days) at room temperature for 1 to 2 hours. The samples were then decanted and the membranes were washed 3×5 min with 2 mL of 1× Wash Buffer I (Ray Biotech) at room temperature with shaking. Then, the membranes were washed 2×5 min with 2 mL of 1× Wash Buffer II (Ray Biotech) at room temperature with shaking. Thereafter, 1 mL of diluted biotin-conjugated antibodies (Ray Biotech) was added to each membrane and incubated at room temperature for 1-2 hours and washed with the Wash Buffers as described above. Diluted HRP-conjugated streptavidin (2 mL) was then added to each membrane and the membranes were incubated at room temperature for 2 hours. Finally, the membranes were washed again, incubated with the ECL™ detection kit (Amersham) according to specifications and the results were visualized and analyzed using the Kodak Gel Logic 2200 Imaging System. The secretion of various angiogenic proteins by AMDACs is shown in FIG. 3.

ELISAs: Quantitative analysis of single angiogenic cytokines/growth factors in cell-conditioned media was performed using commercially available kits from R&D Systems (Minneapolis, Minn.). In brief, ELISA assays were performed according to manufacturer's instructions and the amount of the respective angiogenic growth factors in the conditioned media was normalized to 1×10⁶ cells. Amnion derived adherent cells (n=6) exhibited approximately 4500 pg VEGF per million cells and approximately 17,200 pg IL-8 per million cells.

TABLE 10
ELISA results for angiogenic markers

| AMDAC Marker | Positive | Negative |
|---|---|---|
| ANG | X | |
| EGF | X | |
| ENA-78 | X | |
| FGF2 | X | |
| Follistatin | X | |
| G-CSF | X | |
| GRO | X | |
| HGF | X | |
| IL-6 | X | |
| IL-8 | X | |
| Leptin | X | |
| MCP-1 | X | |
| MCP-3 | X | |
| PDGFB | X | |
| PLGF | X | |
| Rantes | X | |
| TGFB1 | X | |
| Thrombopoietin | X | |
| TIMP1 | X | |
| TIMP2 | X | |
| uPAR | X | |
| VEGF | X | |
| VEGFD | X | |

In a separate experiment, AMDACs were confirmed to also secrete angiopoietin-1, angiopoietin-2, PECAM-1 (CD31; platelet endothelial cell adhesion molecule), laminin fibronectin, MMP1, MMP7, MMP9, and MMP10.

### 6.3 Example 3

### Differentiation of Amnion Derived Adherent Cells

### 6.3.1 Example 3.1

### Osteogenic Non-Differentiation of Amnion Derived Adherent Cells

This Example demonstrates that amnion derived adherent cells (AMDACs) do not differentiate into osteogenic cells, as established by, e.g., von Kossa staining, which stains for mineralization, e.g., calcium deposited by cells.

Cryopreserved OCT-4⁻ AMDACs obtained as described in Example 1, above, were thawed, washed to remove dimethylsulfoxide (DMSO) and re-suspended in growth medium. The cells were seeded at 5000 cells/cm² in 24-well plates and 6-well plates in growth medium and incubated overnight. Subsequently, the medium was removed and replaced with osteogenic medium comprising DMEM-low glucose, 10% v/v fetal bovine serum (FBS), 10 mM beta glycerophosphate (Sigma), 100 nM dexamethasone (Sigma), 100 μM ascorbic acid phosphate salt (Sigma), fungizone (Gibco), 50 units/ml penicillin, and 50 mg/ml streptomycin (Gibco). The osteogenic medium was supplemented with 20 ng/ml transforming growth factor-beta1 (TGF-β1) (Sigma), and 40 ng/ml human recombinant bone morphogenetic protein-2 (BMP-2) (Sigma). Culture of the AMDACs was continued in osteogenic medium for a total of 28 days with media changes every 3-4 days. At the end of the culture period, the cells were collected, washed, and stained as detailed below for evaluation of mineralization, an indicator or osteogenic differentiation. When observed under the microscope, the cell layer was fully confluent with fibroblastoid morphology (e.g., non-cuboidal in appearance), with no nodules observed.

As controls, dermal fibroblasts and bone marrow-derived mesenchymal stem cells (BM-MSCs) were cultured in the osteogenic medium as well. Adult normal human dermal fibroblasts (NHDF) were acquired from Lonza (Walkersville, Md., USA) and neonatal NHDF wew acquired from ATCC (Manassas, Va., USA). Three BM-MSC lines from different origin were evaluated: one from ScienCell Laboratories (Carlsbad, Calif., USA), a second from Lonza (Walkersville, Md., USA), and a third was isolated from fresh whole normal bone marrow aspirates, obtained from AllCells (Emeryville, Calif., USA).

Cells were fixed with 10% (v/v) neutral buffered methanol. After fixation, the cells were washed in deionized water and incubated in 5% Silver Nitrate (Aldrich) for 1 hour under indirect UV light. The cells were then washed in deionized water and incubated in 5% (w/v) sodium thiosulphate for 5 minutes. The cells were then washed again in distilled water and examined by light microscopy.

Differential expression levels of osteogenic differentiation-related genes bone sialoprotein (IBSP) and osteocalcin (BGLAP), before and after induction, were evaluated by RT-PCR. Specifically, the AMDACs were received at the end of the osteogenesis differentiation assay, then lysed using RLT lysis buffer (Qiagen). Cell lysates were stored at −80° C. AMDAC cell lysates were thawed, and RNA was isolated using an RNEasy kit (Qiagen) per manufacturer's instructions with DNAse treatment. RNA was then eluted with DEPC treated water, and the RNA quantity was determined using a Nanodrop ND1000 spectrophotometer. cDNA was made from the RNA using Applied Biosystems reverse transcription reagents. Real time PCR reactions were done using Taqman Universal PCR master mix from Applied Biosystems. Taqman gene expression assays used were Hs00173720 Bone Sialoprotein, Hs00609452 Osteocalcin, and GAPDH. Real time PCR reactions were run in an ABI 7300 system as shown below:

| Stage | Repetitions | Temperature | Time | Ramp Rate |
|---|---|---|---|---|
| 1 | 1 | 50.0° C. | 2:00 | 100 |
| 2 | 1 | 95.0° C. | 10:00 | 100 |
| 3 | 40 | 95.0° C. | 0:15 per | 100 |
| | | 60.0° C. | 1:00 per | 100 |

Interpretation of Threshold Cycle (Ct) values:

Average Ct 1-10 very high expression

Average Ct 10-20 high expression

Average Ct 20-30 medium level expression

Average Ct 30-35 low expression

Average Ct 35-40 very low expression

Expression values (Ct) of each gene were normalized to that of the housekeeping gene GAPDH. The normalized expression values (dCt) of each Sample were then compared pre- and post-induction. The relative differences, in terms of fold-change, were reported as “RQ”. Due to the typical variability in dCt of housekeeping genes, any induction fold difference of less than 3 was not considered to be significant.

Results: Von Kossa staining results demonstrated that AMDACs were clearly nonosteogenic, as no von Kossa staining was detected. Control fibroblasts showed minimal mineralization, while BM-MSC displayed various degrees of mineralization.

TABLE 11
von Kossa Staining Results

| Cell Type | Donor ID | von Kossa Staining Intensity |
|---|---|---|
| AMDAC | 1 | − (Negative) |
| AMDAC | 2 | − (Negative) |
| Dermal Fibroblast | 3 | + (Borderline Positive) |
| adult normal | | |
| Dermal Fibroblast | 4 | − (Negative) |
| neonatal normal | | |
| Bone Marrow MSC | 5 | ++++ (Positive) |
| Bone Marrow MSC | 6 | ++ (Positive) |
| Bone Marrow MSC | 7 | + (Borderline Positive) |

With respect to gene expression, all cells tested displayed moderate basal expression of osteocalcin (Ct 27.5-30.9). AMDACs demonstrated a marginal (<2 fold) induction of osteocalcin expression that was not deemed to be significant when compared to the induction of osteocalcin expression observed for fibroblasts or BM-MSC. As such, the induction of osteocalcin expression by AMDACs was not indicative of osteogenic potential. For bone sialoprotein gene, no expression was found on AMDACs prior to induction, and very low expression was observed post-induction. In contrast, 2 out of 3 BM-MSC lines showed substantial up-regulation upon induction. Variation in BM-MSCs for induction of bone sialoprotein is possibly due to donor variation.

TABLE 12
Gene Expression Results

| | | | | | | | |
|---|---|---|---|---|---|---|---|
| | Donor | | BGLAP | | dCt St. | Fold | GAPDH |
| Cell Type | ID | Condition | Ct | Avg. dCt | Dev. | Induction | Ct |

BGLAP (Osteocalcin)

| | | | | | | | |
|---|---|---|---|---|---|---|---|
| AMDAC | 2 | Basal | 28.2 | 10.2 | 0.07 | 1.6 | 18.0 |
| | | Induced | 29.2 | 9.5 | 0.12 | | 19.7 |
| Fibroblast | 3 | Basal | 28.2 | 10.0 | 0.11 | 0.8 | 18.2 |
| | | Induced | 28.4 | 10.4 | 0.12 | | 18.0 |
| | 4 | Basal | 29.5 | 10.8 | 0.24 | 0.6 | 18.6 |
| | | Induced | 30.7 | 11.6 | 0.18 | | 19.1 |
| BM-MSC | 5 | Basal | 27.7 | 9.9 | 0.09 | 0.3 | 17.8 |
| | | Induced | 30.9 | 11.7 | 0.14 | | 19.1 |
| | 6 | Basal | 27.5 | 9.9 | 0.12 | 0.3 | 17.6 |
| | | Induced | 29.8 | 11.6 | 0.07 | | 18.3 |
| | 7 | Basal | 27.0 | 9.5 | 0.10 | 0.3 | 17.6 |
| | | Induced | 29.8 | 11.0 | 0.16 | | 18.7 |

IBSP (Bone Sialoprotein)

| | | | | | | | |
|---|---|---|---|---|---|---|---|
| AMDAC | 2 | Basal | >40 | 17.7 | 0.10 | 0.13 | 18.0 |
| | | Induced | 38.6 | 20.6 | 0.12 | | 19.7 |
| Fibroblast | 3 | Basal | 35.8 | ND | 0.11 | NA | 18.2 |
| | | Induced | 38.6 | 18.9 | 0.12 | | 18.0 |
| | 4 | Basal | >40 | ND | 0.24 | NA | 18.6 |
| | | Induced | 38.2 | 19.1 | 0.18 | | 19.1 |
| BM-MSC | 5 | Basal | 33.6 | 15.8 | 0.09 | 0.066 | 17.8 |
| | | Induced | 38.9 | 19.7 | 0.14 | | 19.1 |
| | 6 | Basal | 35.7 | 18.1 | 0.12 | 4405 | 17.6 |
| | | Induced | 24.2 | 6.0 | 0.07 | | 18.3 |
| | 7 | Basal | 32.5 | 15.0 | 0.10 | 1508 | 17.6 |
| | | Induced | 23.1 | 4.4 | 0.16 | | 18.7 |

ND—Not detected
NA—Not able to calculate because uninduced condition was not detected (that is, no Ct value was determinable)

Thus, based on the above results, it was concluded that AMDACs do not exhibit osteogenic potential.

### 6.3.2 Example 3.2

### Chondrogenic Non-Differentiation of Amnion Derived Adherent Cells

This Example demonstrates that amnion derived adherent cells, as described herein, do not differentiate along a chondrogenic pathway.

OCT-4⁻ AMDACs as described elsewhere herein were used in a chondrogenesis assay, along with dermal fibroblasts and BM-MSCs as controls. For each test sample, 0.25×10⁶ cells were placed in a 15 mL conical tubes and centrifuged at 200×g for 5 minutes at room temperature to form a spherical pellet. Pellets were cultured either in chondrogenic induction medium (Lonza Chondrocyte Medium (Lonza PT-3003)) containing TGF beta-3 (10 ng/mL), recombinant human growth/differentiation factor-5 (rhGDF-5) (500 ng/mL), or a combination of TGF beta-3 (10 nanogram/milliliter), and rhGDF-5 (500 ng/mL)) or in growth medium (DMEM-low glucose (Gibco)+FBS (2% v/v) (Hyclone)+Penicillin and Streptomycin) for three weeks. During culture, full exchanges of media were performed twice a week.

At the end of the culture period, cell pellets were fixed in 10% formalin for 24 hours. All samples were then dehydrated through graded alcohols and were embedded in paraffin. Sections were cut to a thickness of 5 μm and then stained according to protocols as described below. The histological sections were examined using light microscopy.

Alcian Blue Staining: When used in a 3% acetic acid solution (pH 2.5), Alcian Blue stains both sulfated and carboxylated acid mucopolysaccharides and sulfated and/or carboxylated sialomucins. 1% Alcian Blue (Sigma-Aldrich #23655-1) in 3% Acetic Acid was used, followed by a 0.1% Nuclear fast red (Sigma-Aldrich #22911-3) counterstain. In brief, the sections were deparaffinized and hydrated through graded alcohols to distilled water, stained in Alcian Blue for 30 minutes, washed in running tap water for two minutes, rinsed in distilled water, then counterstained in nuclear fast red solution for 5 minutes, washed in running tap water for 1 minute, dehydrated through graded alcohols, cleared in xylene and finally mounted with resinous mounting medium.

Type II Collagen Staining: The presence of Type II Collagen in cell culture samples before and after chondrogenic differentiation conditions are evaluated by immunohistochemistry as outlined below. Collagen II production by the cells was assessed using antibody 5B2.5 (Abcam Cat. # ab3092), a mouse monoclonal highly specific to type II collagen and which displays no cross reaction with types I, III, IV, V, VI, IX, X, or XI collagens, and no cross-reaction with pepsin-digested type II collagen. The assay used goat anti-mouse AF 594 (Invitrogen IgG2a, Cat#A21135) as a secondary antibody. Cell pellets were fixed in 10% formalin for a minimum of 4 hours to overnight and were infiltrated in paraffin.

All cell samples were washed in PBS and exposed to protein blocking solution containing PBS, 4% goat serum and 0.3% Triton-100× for 30 minutes at room temperature. Primary antibodies diluted in blocking solution (1:50 and 1:100) were then applied overnight at 4° C. Next morning, samples were washed in PBS, and secondary antibodies (goat-anti-mouse AF594) diluted in blocking solution (1:500) were applied for 1 hr at room temperature. The cells were then washed in PBS and 600 nM DAPI solution was applied for 10 minutes at room temperature to visualize nuclei.

BM-MSCs and fibroblasts formed cell pellets in chondrogenic induction medium. Chondrocytes formed large cell pellet with no distinct cell populations apically or centrally. In contrast, AMDACs failed to form a cell pellet during the culture period. No staining results were obtained for AMDACs for either collagen II or Alcian Blue because AMDACs failed to form cell pellets. Therefore, it was concluded that AMDACs are non-chondrogenic.

### 6.3.3 Example 3.3

### Neural Differentiation of Amnion Derived Adherent Cells

This Example demonstrates that amnion derived adherent cells can be differentiated to cells with characteristics of neural cells. Neural differentiation of the AMDACs was compared to that of normal human neuroprogenitors (Lonza), dermal fibroblasts, neonatal normal (Donor 3), Bone Marrow MSC (Donors 5 and 6).

In a first short term neural differentiation procedure, AMDACs and the other cells were thawed and expanded in their respective growth media after seeding at about 5000/cm² until they were sub confluent. Cells were trypsinized and seeded at 6000 cells per well in tissue culture-coated plate. All cells were initially expanded for 4 days in DMEM/F12 medium (Invitrogen) containing 15% v/v FBS (Hyclone), with basic fibroblast growth factor (bFGF) at 20 ng/ml, epidermal growth factor (EGF) at 20 ng/ml (Peprotech) and Penicillin/Streptomycin (PenStrep, Invitrogen). After 4 days, the cells were rinsed in PBS (Invitrogen). The cells were then cultured in DMEM/F12 with 20% v/v FBS, PenStrep for about 24 hours. After 24 hours, the cells were rinsed with PBS (Invitrogen) and cultured in induction medium consisting of DMEM/F12, serum free, containing 200 mM butylated hydroxyanisole, 10 nM potassium chloride, 5 mgs/mL insulin, 10 nM forskolin, 4 nM valproic acid, and 2 nM hydrocortisone (Sigma). The cells were subsequently fixed at −20° C. with 100% methanol. Fixed samples were then evaluated by immunohistochemistry (IHC) for expression of human nestin using an anti-nestin antibody (Alexa-Fluor 594 (Red) conjugated), with counterstaining with DAPI for nuclei.

In a second short term neural differentiation protocol, all cells were initially expanded for 4 days in DMEM/F12 medium (Invitrogen) containing 15% FBS (Hyclone), with basic FGF at 20 ng/ml, EGF at 20 ng/ml and PenStrep (Invitrogen). After 4 days, the cells were rinsed in PBS (Invitrogen) and were cultured in DMEM/F12 with 20% v/v FBS, PenStrep. After 24 hrs, cells were rinsed with PBS. The media were then switched to Neural Progenitor Expansion medium (NPE), which comprised NEUROBASAL™-A basal medium (Gibco), with B27 (Gibco), 4 mM L-glutamine, 1 μM retinoic acid (Sigma), and PenStrep. After four days, the medium was removed from each well and cells were fixed with ice cold 4% w/v paraformaldehyde for 10 minutes at room temperature. Fixed samples were then evaluated by IHC for expression of GFAP (glial fibrillary acidic protein) for astrocyte phenotype, and TuJ1 (neuron-specific class III tubulin) for neuronal phenotype, respectively.

In the first differentiation protocol, all cell types transformed into a cell type with bipolar morphology and stained positive with nestin. Neuroprogenitors constitutively expressed nestin as expected. In the second differentiation protocol, expression of neuronal-related (Tuj1) and astrocyte-related (GFAP) markers were evaluated. Upon induction, AMDAC, and BM-MSC expressed low levels of Tuj1. Expression on fibroblasts was found to be borderline positive which could be due to background. AMDACs, and one BM-MSC cell line, exhibited low-level expression of GFAP. The positive control cell line (neuroprogenitors) constitutively expressed both Tuj1 and GFAP, as expected.

Thus, AMDACs are able, under neural inducing conditions, to exhibit morphological and biochemical changes consistent with neural differentiation.

### 6.4 Example 4

### Immunomodulation Using Amnion Derived Angiogenic Cells

This example demonstrates that AMDACs display immunosuppressive function in vitro in an assay utilizing bead-stimulated T cells.

6.4.1 AMDAC-Mediated Suppression T Cell Proliferation

AMDACs were obtained as described in Example 1, above. CD4⁺ and CD8⁺ T cells were obtained from human peripheral blood.

The T cells were labeled with carboxyfluorescein succinimidyl ester (CFSE) and mixed with anti-CD3 anti-CD28-coated Dynabeads, followed by culture in the absence of the AMDACs or a coculture with the AMDACs in a manner that allowed cell to cell contact, also known as a Bead T-lymphocyte reaction (BTR). Coculture with the AMDACs was performed by mixing 100,000 T-lymphocytes with anti-CD3 and anti-CD28 coated DynaBeads (Invitrogen) at a bead:T-lymphocyte ratio of 1:3 in a well of a 96-well plate, in the presence or absence of 20,000 AMDAC cells. The mixed (coculture) and unmixed cell cultures were incubated at 37° C., 5% CO₂, and 90% relative humidity for 5 days. Normal human dermal fibroblasts (NHDF), which do not possess substantial T cell inhibitory activity were used as a negative control, and subjected to the same conditions as the AMDACs.

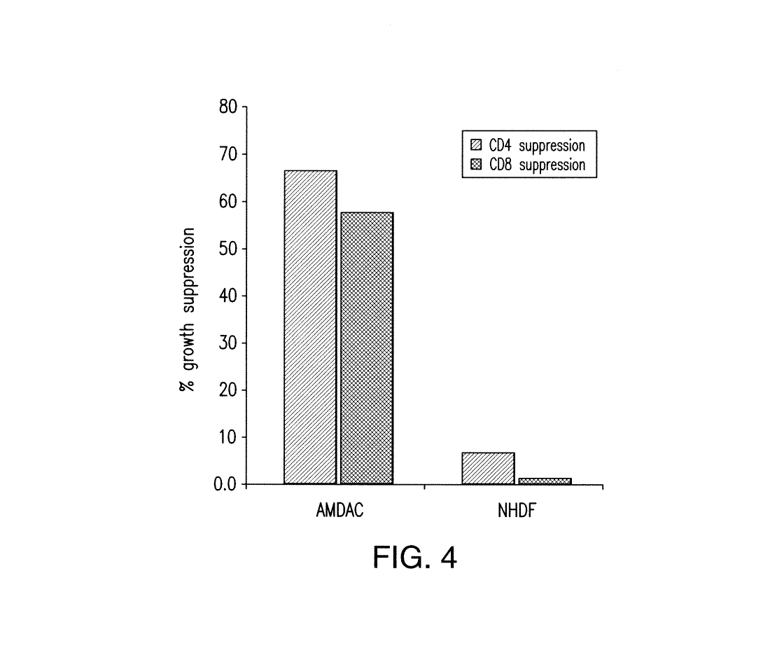

Following the 5 days, CFSE fluorescence on the CD4+ and CD8+ T cells was detected using flow cytometry, and the percentage of suppression of T cell growth was calculated based on the increased fraction of non-proliferated (CFSE high) T cells compared to the culture of CFSE-labeled T cells that were not co-cultured with AMDACs or NHDF. As demonstrated in FIG. 4, AMDACs inhibit the proliferation of CD4⁺ and CD8⁺ T cells in vitro, indicating that AMDACs are immunomodulatory.

6.4.2 Media Conditioned by AMDACs Inhibits Secretion of TNF-Alpha by T Cells

AMDACs were obtained as described in Example 1, above. T cells were obtained from human peripheral blood.

The AMDACs were seeded on tissue culture plates and incubated overnight to form an adherent monolayer. The next day, the AMDAC culture was stimulated with IL-1 beta, which has previously been shown to be a potent inducer of AMDAC-derived anti-inflammatory factors. After 16 h of IL-1 beta stimulation, the medium conditioned by the AMDACs was collected and mixed at a 9:1 volume ratio with human peripheral blood T cells coated with anti-CD3 anti-CD28-coated Dynabeads. A separate population of human peripheral blood T cells coated with anti-CD3 anti-CD28-coated Dynabeads was maintained as a control. The T cells mixed with AMDAC-conditioned medium and the unmixed population of T cells were incubated at 37° C., 5% CO2, and 90% relative humidity for 72 h. Medium conditioned by normal human dermal fibroblasts (NHDF), which do not possess substantial TNF-alpha inhibitory activity was used as a negative control, and subjected to the same conditions as the AMDACs.

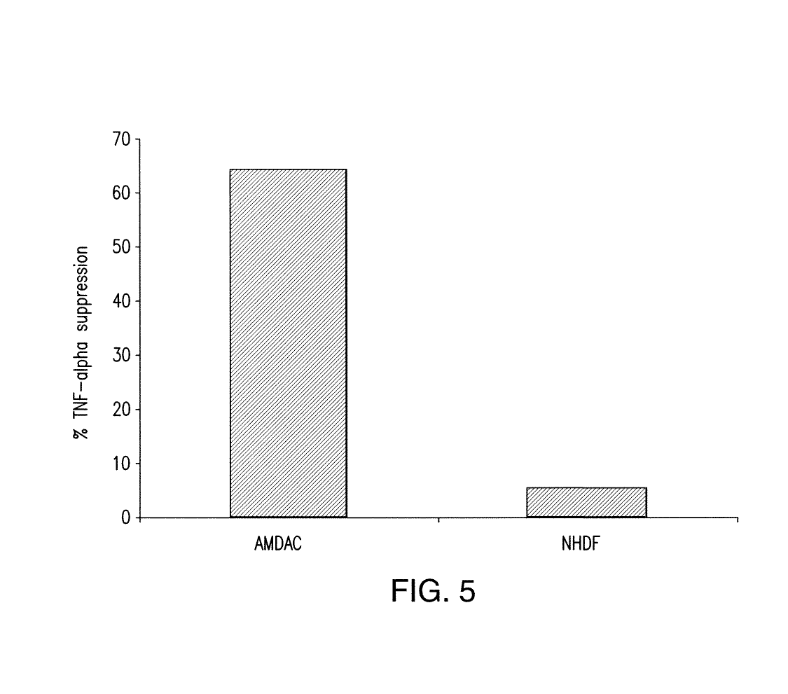

Following the 72 h culture, the concentration of T-cell derived TNF-alpha was measured in the T cell culture supernatants using a cytometric bead-based ELISA method. The percent suppression of TNF-alpha secretion was calculated based on the decrease of TNF-alpha concentration in the presence of AMDAC-conditioned medium compared to the control T cell culture which was not mixed with AMDAC-conditioned medium. As demonstrated in FIG. 5, the culture of the T cells in the presence of AMDAC-conditioned medium induced the suppression of production of T cell derived TNF-alpha.

### 6.5 Example 5

### AMDACS Modulate the T Cell Compartment

This Example demonstrates that amnion derived adherent cells (AMDACs), obtained as described in Example 1, are able to influence skewing in the Th1, Th17 and FoxP3 T_{reg} subsets.

6.5.1 Methods

### T-Lymphocyte Proliferation Assays

Mixed lymphocyte reactions (MLR) were performed by mixing 100,000 HLA-mismatched carboxyfluorescein succinimidyl ester (CFSE)-labeled T-lymphocytes with 10,000 mature dendritic cells (mDC) in each well of a FALCON flat bottom 96 well tissue culture plate (Fisher Scientific, Pittsburgh, Pa.) in the presence or absence of 20,000 AMDAC cells, isolated as described in Example 1, above. The mixed cell culture was incubated at 37° C., 5% CO₂, and 90% relative humidity for 6 days. At day 6 all cells were recovered and stained with anti-CD4-PE and anti-CD8-APC (R&D systems, Minneapolis, Minn.).

Bead T-lymphocyte reactions (BTR) were performed by mixing 100,000 T-lymphocytes with anti-CD3 and anti-CD28 coated DynaBeads (Invitrogen) at a bead:T-lymphocyte ratio of 1:3 in a well of a 96-well plate. The BTR reaction was performed in the presence or absence of 20,000 AMDAC cells. The mixed cell culture was incubated at 37° C., 5% CO₂, and 90% relative humidity for 6 days. At day 6 all cells were recovered and stained with anti-CD4-PE and anti-CD8 APC (R&D systems, Minneapolis, Minn.).

T-lymphocyte proliferation was measured by analysis of CFSE fluorescent intensity on CD4 and CD8 single positive cells with a FACS Canto II machine (BD, Franklin Lake, N.J.). All FACS data in this study were analyzed by using flowjo 8.7.1 software (Tree Star, InC. Ashland Oreg.).

### T Cell Skewing (Polarization)

Th1 skewing was carried out using BTR reactions with an additional Th1 skewing cytokine cocktail containing IL-2 (200 IU/ml), IL-12 (2 ng/ml) and anti-IL-4 (0.4 μg/ml).

For Th17 skewing, 5×10⁵ total T-lymphocytes were stimulated with 5×10⁵ sorted CD14⁺ monocytes, 50 ng/mL anti-CD3 antibody (BD BioSciences) and 100 ng/mL LPS (Sigma Aldrich) in either the presence or absence of 50,000 AMDACs for 6 days. The Th17 cell population was analyzed by intracellular cytokine staining (ICCS) staining of IL-17 on the CD4 positive population.

### Intracellular Cytokine and Foxp3 Staining

The Th1 cell subset was enumerated as follows. T cells from BTR reactions were re-activated with 50 ng/mL phorbol myristate acetate (PMA) and 750 ng/mL ionomycin (PI) (Sigma Aldrich) for 5 hours. GOLGISTOP™ (Becton Dickinson; a protein transport inhibitor) was added during the last 3 hours. Cells were then surface stained with PE labeled anti-CD4 antibody and subsequently with APC conjugated anti-IFN-γ antibody with the Cytofix/Cytoperm kit (Becton Dickinson) according to the manufacturer's instructions.

In order to enumerate the Th17 cell subset, T cells from a Th17 skewing activation reaction were re-activated with 50 ng/mL PMA and 750 ng/mL ionomycin (Sigma Aldrich) for 5 hours with GOLGISTOP™ (Becton Dickinson) present during the last 3 hours. Cells were then stained with PE labeled anti-CD4 antibody and subsequently with APC conjugated anti-IL-17 antibody with the Cytofix/Cytoperm kit (Becton Dickinson) according to the manufacturer's instructions.

In order to enumerate the Treg cell subset, T cells from BTR reactions were surface stained with PE labeled anti-CD4 antibody and subsequently with APC conjugated anti-Foxp3 antibody using the Foxp3 staining kit (eBioscience, San Diego, Calif.) according to the manufacturer's instructions.

### Dendritic Cell Differentiation and Stimulation

Immature DC (iDC) were generated from a magnetically sorted CD14⁺ monocyte population by mitogen-directed differentiation. Briefly, iDCs were obtained from monocytes cultured at 1×10⁶/ml with GM-CSF (20 ng/ml) and IL-4 (40 ng/ml) for 4 days. iDCs (1×10⁵ cells) were then stimulated with 1 μg/ml LPS for 24 hours in either the absence or presence of 1×10⁵ AMDACs in each well of a FALCON 24 well tissue culture plate (Fisher Scientific, Pittsburgh, Pa.). Culture supernatant was collected and the cytokine profile was analyzed by Cytometric Bead Array (CBA).

### Cytometric Bead Array (CBA) Analysis

Cytokine concentrations were measured in culture supernatants using the Cytometric Bead Array system (CBA; Becton Dickinson) for the simultaneous quantitative detection of multiple soluble analytes according to the manufacturer's instructions. Briefly, samples of BTR culture supernatants were incubated with a mix of capture beads for specific detection of the following cytokines produced by activated T cells: IL-2, IL-4, IL-5, IL-10, TNF, lymphotoxin-alpha (LT-α) and IFN-γ. Subsequently, bead bound cytokines were coupled with fluorescently labeled detection reagents and detected using the FACSCanto II flow cytometer following the manufacturer's protocols. Data was acquired and analyzed using the FACS-DIVA 6.0 software (Becton Dickinson), followed by calculation of cytokine concentrations using the FCAP Array 1.0 program (Becton Dickinson).

### IL-21 ELISA

Soluble IL-21 was measured in supernatant obtained from Th17 skewing cultures with the IL-21 ELSAI kit from eBioscience (88-7216) according to the manufacturer's protocol.

### NK Proliferation Assay and NK Cytotoxicity Assay

Human NK cells were isolated from PBMC using an NK cell isolation kit (Miltenyi Biotech, Aubum, Calif.) according to the manufacturer's instructions. NK cell proliferation was determined by culturing 2.5×10⁵ NK cells in 1 ml IMDM containing 10% fetal bovine sera (FBS) (Hyclone) supplemented with 35 μg/ml transferrin, 5 μg/ml insulin, 20 □M ethanolamine, 1 μg/ml oleic acid, 1 μg/ml linoleic acid, 0.2 μg/ml palmitic acid, 2.5 μg/ml BSA, 0.1 μg/ml PHA (Sigma-Aldrich) and 200 IU/ml human IL-2 (R&D), together with mitomycin C treated (16 g/ml) feeder cells (either 1×10⁶ human allogeneic PBMC or 1×10⁵ K562 cells). Cells were incubated at 37° C. in 5% CO₂ with the addition of an equal volume of IMDM (10% FBS, 2% human serum and 400 IU/ml IL-2) every 3 days. NK cell number was determined by FACS every seven days as follows. Briefly, total NK cells were collected from the tissue culture well. After washing with PBS, cells were then stained with 2 uM TO-PRO3. Finally, 10 μl counting beads (Spherotech, Cat# ACBP-50-10) were added to each sample which served as an internal standard for calibration of total cell number. Relative NK number was calculated based on the number of total live NK cells per 1000 counting beads collected.

The NK cytotoxicity assay was carried out by mixing NK cells with target cells at different effector/target (E/T) ratios. After overnight culture, target cell numbers were determined using the counting beads method described above plus cell surface markers to differentiate NK cells from target cells. For NK cytotoxicity of K562 cells, FITC conjugated anti-HLA-ABC antibodies were used as the NK cell marker, because K562 cells are HLA-ABC negative. For AMDAC cells, CD90-PE was used to distinguish AMDACs from NK and K562 cells. Percent cytotoxicity was calculated as (1−total target number in sample÷total target cells in a control containing no NK cells)×100.

6.5.2 AMDACs Skewing of T Cell Compartment

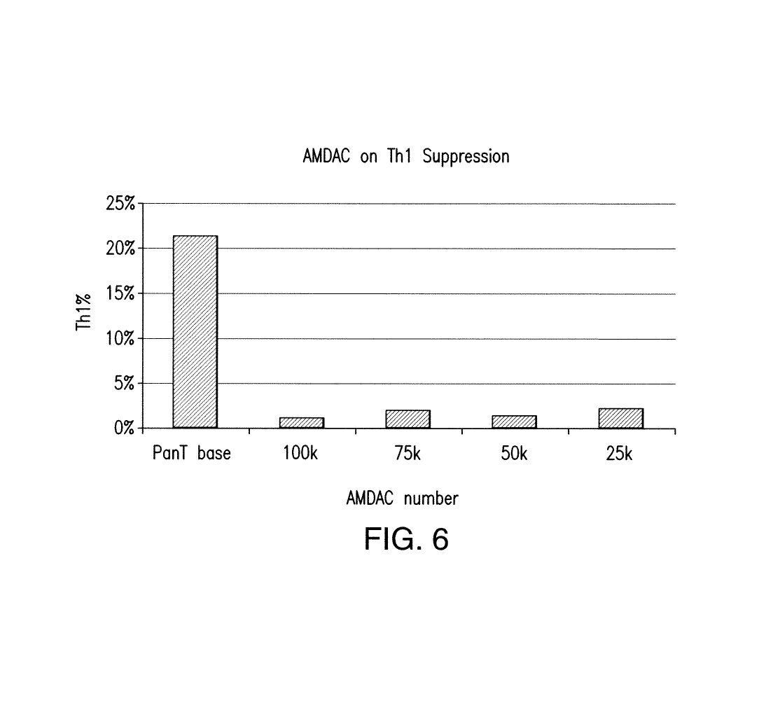

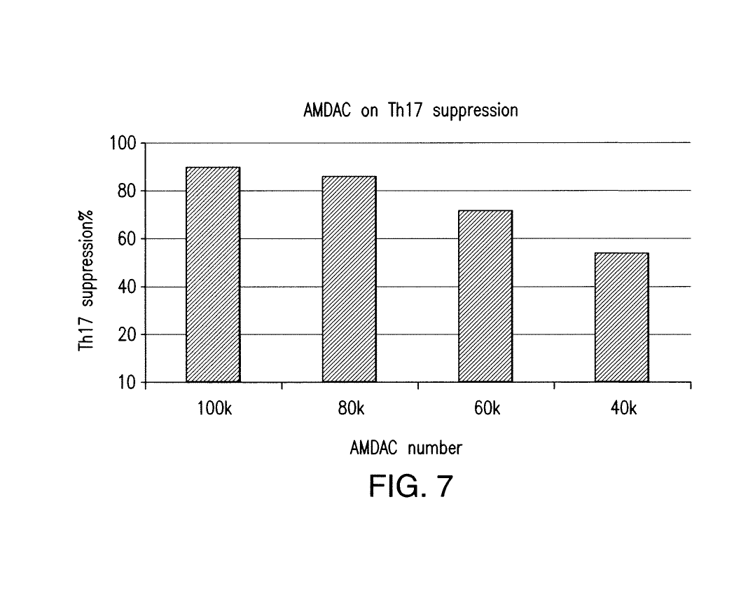

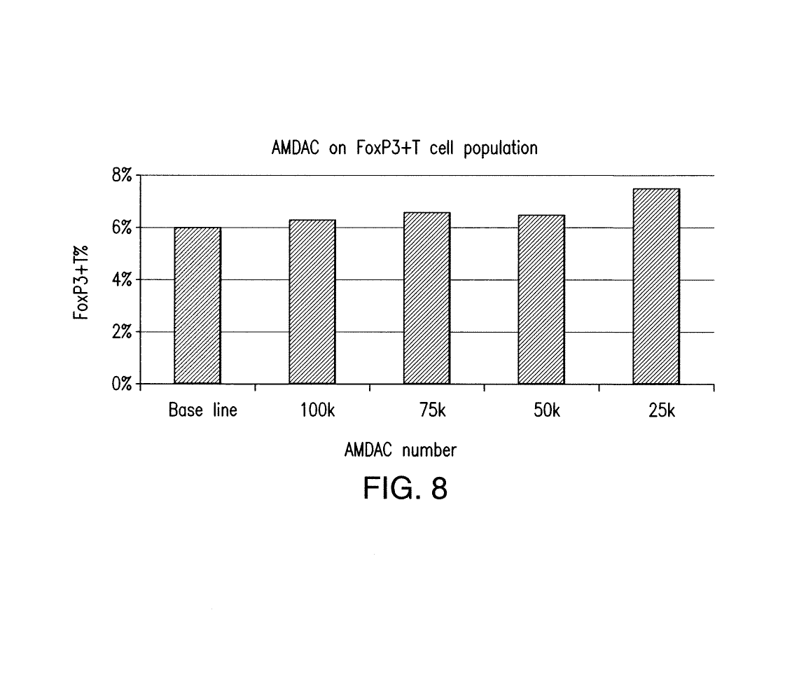

The ability of AMDACs to influence skewing in the T cell compartment was examined by measuring cytokine producing T cells in Th1 and Th17 skewing assays using T cell and AMDAC co-cultures. Briefly, in the Th1 skewing assay, AMDACs were pre-plated. The following day, 1×10⁶/ml T cells, Dynabeads at 6×10⁵/ml, IL-2 (200 IU/ml), IL-12 (2 ng/ml), and anti-IL-4 (0.4 μg/ml) were added and mixed with the AMDACs. Four days later, the percentage of Th1 cells was analyzed by interferon-gamma (IFN-γ) intracellular staining. As shown in FIG. 6, AMDACs greatly reduced the Th1 percentage in a dose dependent manner. Similarly, in a Th17 skewing assay, AMDACs were pre-plated overnight. A mixture of T cells (1×10⁶/ml), CD14⁺ cells (1×10⁶/ml), anti-CD3 (50 ng/ml) and bacterial lipopolysaccharide (LPS) (100 ng/ml) was then added to the plate containing AMDACs. After a six day culture, the Th17 percentage was examined by IL-17 intracellular staining. As shown in FIG. 7, AMDACs suppressed the Th17 percentage in a dose dependent manner. To investigate the effect of AMDACs on a FoxP3 positive T cell population, 1×10⁶ PBMC were co-cultured with AMDACs for 6 days. The FoxP3 positive population was analyzed by FoxP3 intracellular staining. As shown in FIG. 8, AMDACs slightly increased the FoxP3 positive T cell population.

6.5.3 AMDAC Mediated Modulation of DC Maturation and Function

This experiment demonstrates that AMDACs modulate the maturation and differentiation of immature dendritic cells (DCs).

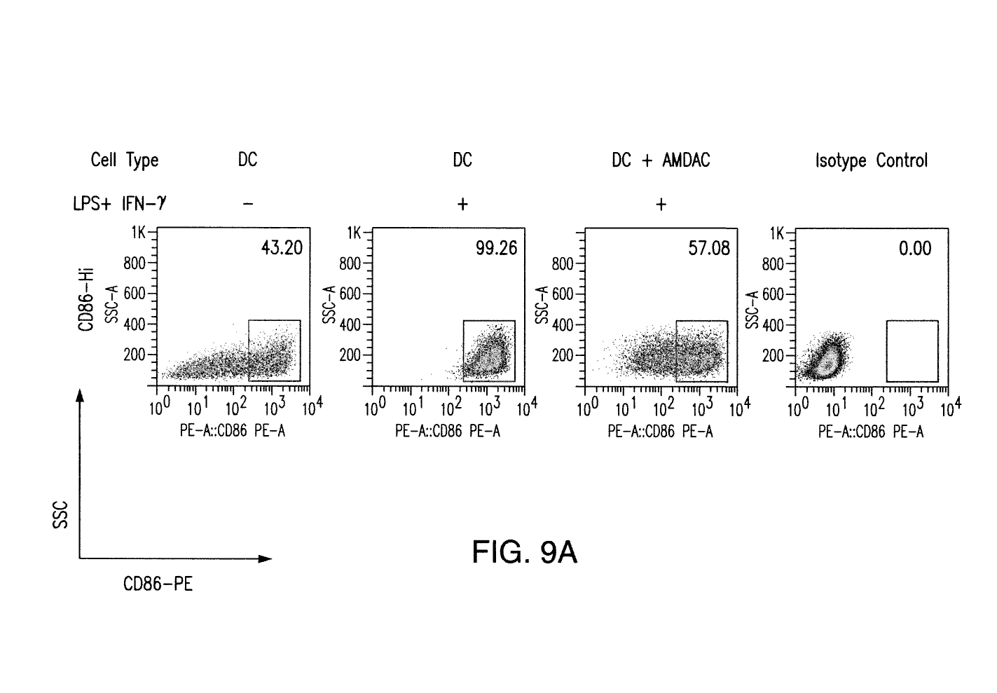

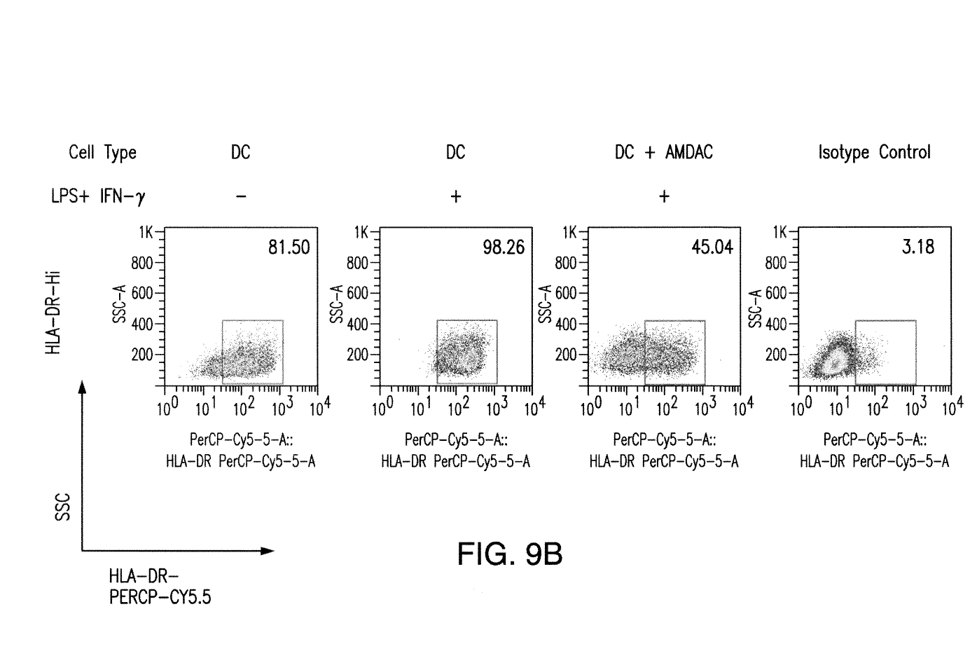

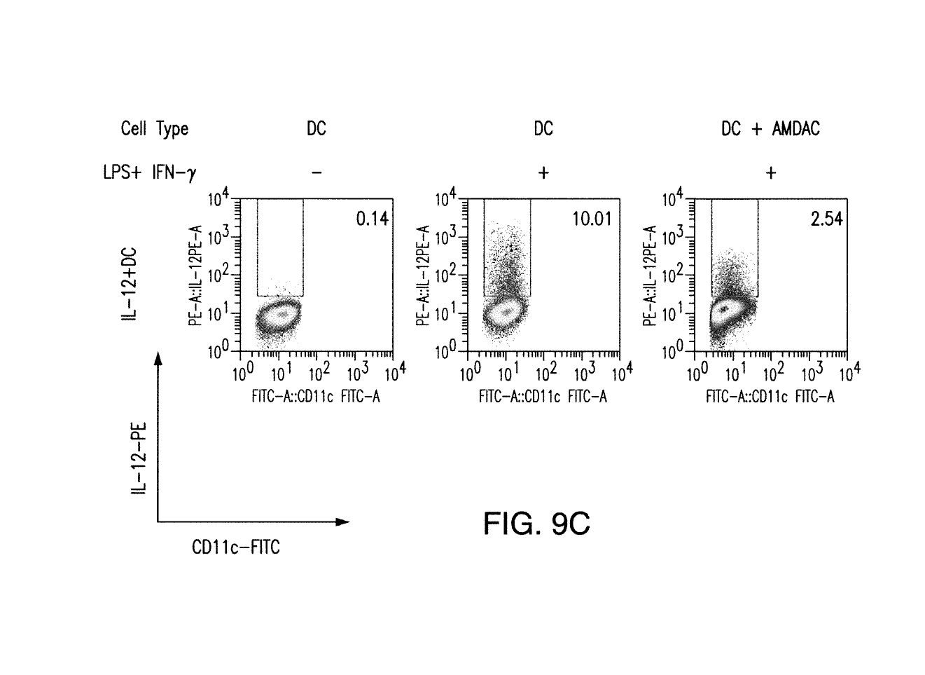

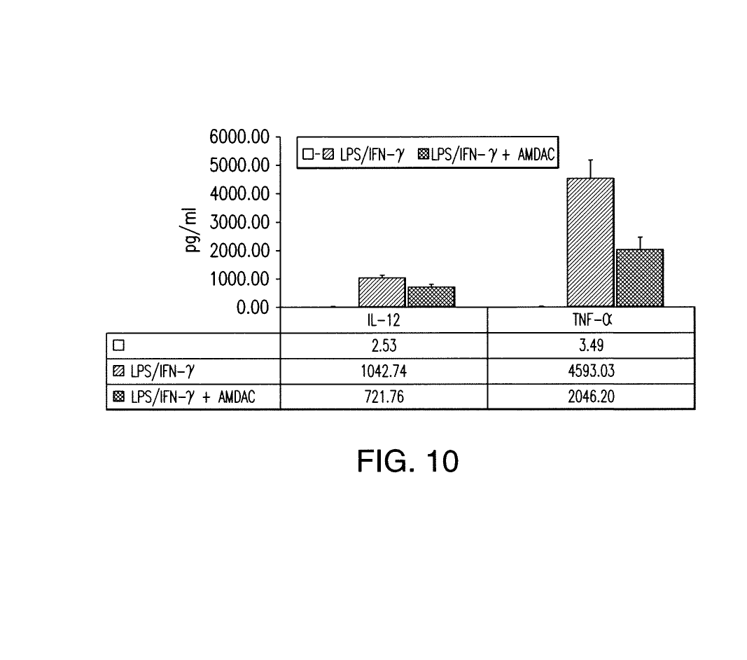

To explore the AMDAC mediated modulation of DC maturation and function, monocyte derived immature DCs were treated with LPS alone or a combination of LPS plus IFN-γ in the absence or presence of AMDACs to further drive the DC maturation process. DC maturation was analyzed by FACS staining of DC maturation markers CD86 and HLA-DR. DC function was assessed by intracellular staining of IL-12 and measurement of soluble cytokine production by CBA. As shown in FIGS. 9A and 9B, AMDACs strongly suppressed LPS and LPS plus IFN-γ-induced DC maturation by down-modulation of CD86 (FIG. 9A) and HLA-DR expression (FIG. 9B) on DCs. Further, as shown in FIG. 9C, AMDACs significantly suppressed the LPS plus IFN-γ-stimulated IL-12-producing DC population by ˜70%. AMDACs were further found to be able to suppress TNF-α and IL-12 production by LPS-stimulated DCs. See FIG. 10.

6.5.4 AMDACs Suppress IL-21 Production in a Th17 Skewing Culture

IL-21 is an important cytokine required for maintenance of a Th17 population. To investigate whether AMDACs are able to modulate IL-21 production, AMDACs were introduced into a Th17 skewing culture as described in the Methods section. AMDACs suppressed IL-21 production by 72.35% in AMDAC-Th17 co-cultures in comparison to a Th17 skewing culture without AMDAC cells. AMDACs also suppressed Th17 T cell numbers by 72.65% as compared to culture in the absence of AMDACs.

6.5.5 AMDAC Modulation of NK Cell Cytotoxicity and Proliferation

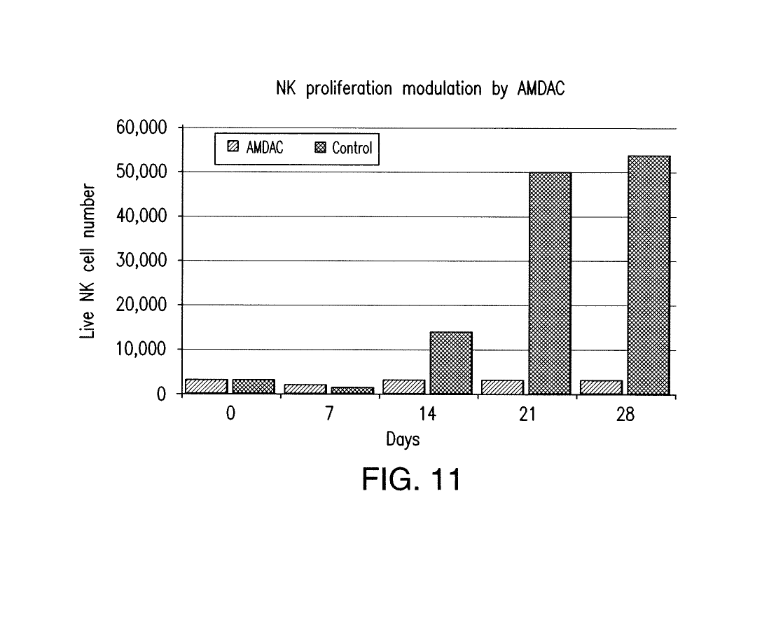

NK cells are a type of cytotoxic lymphocyte that constitutes a major component of the innate immune system. NK cells play a major role in the rejection of tumors and cells infected by viruses as well as allogeneic cells and tissues. To investigate the immunomodulatory effect of AMDACs on NK cells, NK cell proliferation and cytotoxicity assays were established. As shown in FIG. 11, AMDACs suppressed human NK cell proliferation in comparison to a control having no AMDAC cells.

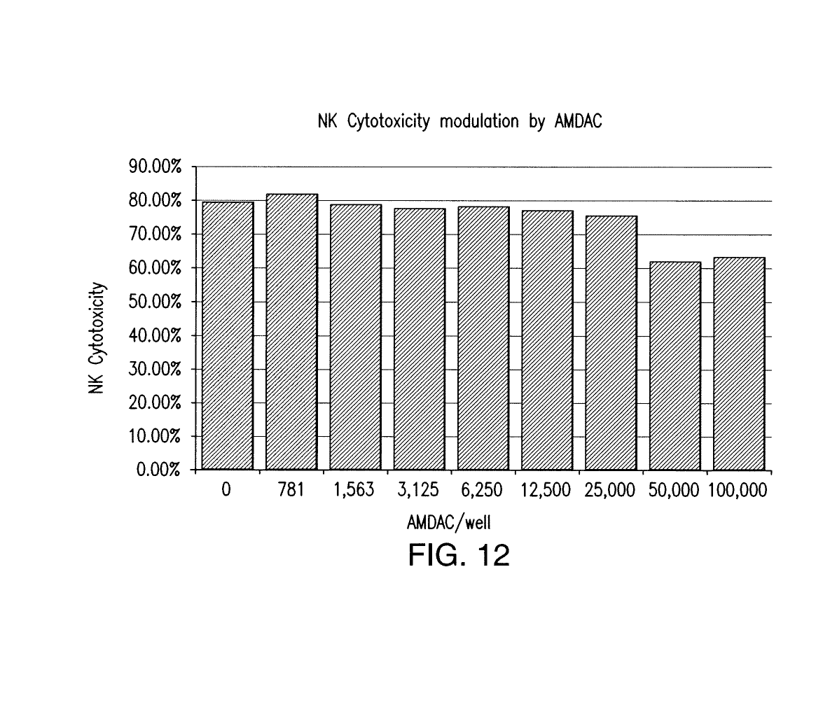

In addition, the effect of AMDACs on NK cell cytotoxicity was investigated. In this assay, AMDACs were introduced into an NK cytotoxicity assay as described in the Methods section above. Briefly, 1×10⁶ NK cells were mixed with 1×10⁵ K562 cells (E/T ratio of 10:1) with a 2 fold titration of pre-seeded AMDACs (1×10⁵ cells). The NK cells and K562 cells were co-cultured overnight, and NK cell cytotoxicity was determined according to the protocol described in the Methods section above. As shown in FIG. 12, AMDAC cells suppressed human NK cell cytotoxicity in a dose dependent manner.

### 6.6 Example 6

### Treatment of SCI Using AMDACS in a Rat SCI Model

This Example provides an exemplary model and method for evaluating the effects of AMDACs on a spinal cord injury and, in particular, for evaluating the immune rejection, migration, and differentiation, of AMDACs transplanted to the uninjured and injured spinal cord of rats. The model provides for the assessment of the effects of AMDACs administration alone or in combination with secondary treatment options, e.g., co-administration with methylprednisolone, lithium, and/or cyclosporin A. The effects of AMDACs on function, including recovery of walking (BBB scores), regeneration of corticospinal tract and serotonergic axons, and white matter area in the spinal cord, are assessed at 12 weeks after injury, with and without cyclosporin, compared to control rats without cell transplants. The cells are transplanted into the spinal cord shortly, 2 weeks, and 6 weeks after injury, to simulate transplantation of cells into the acute, subacute, and chronic phase of spinal cord injury. The survival, migration, and differentiation of AMDACs administered at 0, 1, 2, 3, 4, and 6 weeks after injury are assessed. In addition, expression of neurogenic growth factors, e.g., neurotrophins, following the administration of AMDACs can be assessed utilizing gene chip, RT-PCR and ELISA methodology.

### Experimental Design

In vivo persistence of AMDACs. AMDACs are injected into the central gray region at the upper edge of T9 and lower edge of T10 vertebral segment of the rat spinal cord at 0, 1, 2, 3, 4, and 6 weeks with or without infliction of spinal cord injury with a 25 mm weight drop (n=4/group). After 6 weeks, rats are anesthetized with 60 mg/kg pentobarbital, perfused with formaldehyde, and the spinal cords are sectioned horizontally and examined with an epifluorescent dissecting microscope. The distribution of AMDACs at various distances from the injections sites are measured via fluorescence, and sections are stained immunohistologically for beta-3-tubulin (neuron), GFAP (astrocyte), nestin (progenitor) markers.

Treatments. Rats administered with AMDACs are treated with methylprednisolone (MP, 30 mg/kg bolus at the time of transplant), lithium (Li, 100 mg/kg/day for 6 weeks), and cyclosporin (CsA, 10 mg/kg/day) and the number, distribution, and characteristics of the transplanted AMDACs at 6 weeks after injury and transplantation are assessed. The effects of AMDACs alone, MP alone, Li alone, CsA alone, or MP+Li are assessed. To quantify the cells, the amounts of human DNA and green fluorescent protein (GFP) in the spinal cord are measured. Short-medium-term GFP expression in AMDACs is achieved by Amaxa-based electroporatation of a plasmid vector encoding a constitutive GFP expression cassette. Longer-term expression is achieved by the use of a lentiviral vector encoding constitutive GFP expression.

Gene/Protein Expression. RT/PCR and ELISA is used to measure mRNA and protein levels of LIF, BDNF, GDNF, NT3, NGFA, and GFP in animals that are not treated or treated with AMDACs alone, AMDACs plus MP, AMDACs plus MP and Li, and AMDACs plus MP, Li and CsA.

Recovery/Regeneration. AMDACs are transplanted 2 weeks and 6 weeks after injury with or without CsA, and the animals are kept for 12 weeks. Locomotor recovery (BBB) is assessed and histological studies are performed.

### Protocol

Anesthesia. Sprague-Dawley rats which are 77±1 day old are subjected to laminectomy. The rats are anesthetized with intraperitoneal pentobarbital (45 mg/kg female, 65 mg/kg male). Rats that do not become deeply anesthetized within five minutes are excluded from the experiment. For delayed transplants of cells into spinal cord at 1 week and 4 weeks after injury, the rats are anesthetized by spontaneous respiration of isoflurane via a head-cone (5% induction for 5 minutes and then 1% maintenance).

Spinal Cord Injury. After shaving the rats and preparing the surgery site with betadine, a midline dorsal incision is made to expose the T8-11 vertebral column and a T9-10 laminectomy is carried out to expose the underlying T13 spinal cord. The rats are suspended with clamps placed on the TS and T11 dorsal processes. At one hour after induction of anesthesia, a 10-gram rod is dropped 25 mm onto T13 spinal cord. A thin (100 u) sheet of polylactic acid and polycaprilactone is placed over the dura to prevent adhesions, and a piece of autologous subcutaneous fat is placed on the laminectomy site to retard scar formation. Muscle is sutured at the midline with silk above and below the laminectomy. Skin is closed with stainless steel clips. The clips are removed a week later.

Cell Transplantation. The dura is incised with a 26-gauge tuberculin syringe and a 1-microliter suspension of 200,000 cells is injected into the spinal cord. For delayed transplantation, the laminectomy site is reopened after anesthesia with isoflurane, a small dural incision is made, and a micropipette is used to inject two 1-microliter suspensions of 200,000 cells into the spinal cord rostral and caudal to the impact site.

Postoperative care. The rats are maintained on heating pads until they wake up. Rats showing cyanosis (from the color of their feet) receive transoral tracheal suction to clear secretions and stimulate respiration. Atropine at 0.04 mg/kg IM or glycopyrolate at 0.5 mg/kg IM is optionally administered to reduce intraoperative secretion build up if there are more incidents of respiratory obstruction. Rats showing signs of dehydration (e.g., the skin of the back is pinched and does not settle down in a second) receive 5-10 ml subcutaneous saline injection (5 ml female, 10 ml male). All rats receive 50 mg/kg of cefazolin subcutaneously daily for 7 days, to reduce urinary tract and wound infections.

Postoperative analgesia. Spinal cord injured rats generally do not show evidence of pain because the injury causes anesthesia at and below the injury site. However, for animals subjected to laminectomy only, i.e., without spinal cord injury, and showing postoperative pain, a local anesthetic, Bupivacaine (Marcaine) is administered at the surgical site at a maximum dose of 2 mg/kg body weight. Each animal is monitored for evidence of pain and additional pain relief is provided as needed.

Long-term care. Rats are inspected daily and assessed weekly for locomotor scores (BBB). First, the animals are inspected twice daily and manually expressed if palpation indicates >1 ml urine in their bladders. Rats with cloudy and bloody urine, indicative of bladder infection, after initial 7 day period receive 2.5 mg/kg/day of Baytril (a fluoroquinolone antibiotic) for 7-10 days. If this does not clear up the infection, the rats are euthanized. Second, the rats are kept on sterile white paper litter (Alpha Dry), which keeps the rats dry and shows presence of hemorrhagic urine. Rats with hemorrhagic urine are set aside and cared for in isolation from other rats, to avoid transferring infections. Third, if the rats show evidence of pain (vocalization, sensitivity to touch) or autophagia (biting of the dermatomes below the injury site manifested by hair loss or skin penetration), the rats are given daily oral acetaminophen (64 mg/kg/day “Baby Tylenol” orally) until their skin lesions are completely healed. If no correctable causes of the pain are found, the rats are euthanized. The animals are weighed daily for the first week and weekly thereafter.

Euthanasia. All animals are deeply anesthetized with pentobarbital (100 mg/kg female-male doses) and decapitated for molecular studies or perfused with 4% paraformaldehyde solutions for fixation and histology study.

### 6.7 Example 7

### Treatment of TBI Using AMDACS in a Rat TBI Model

This Example provides an exemplary model and method for evaluating the effects of AMDACs on a traumatic brain injury. Without intending to be bound to any particular theory or mechanism of action, it is believed that traumatic brain injury results in a decrease in splenic mass that correlates with an increase in circulating immune cells leading to increased blood brain barrier permeability. Thus, this method provides for the assessment of the ability of AMDACs to modulate immunologic response; to co-localize with splenocytes to promote splenocyte proliferation and secretion of anti-inflammatory cytokines such as IL-4 and IL-10; preserve splenic mass; and to maintain the integrity of the blood brain barrier following induced traumatic brain injury.

### In Vivo Methods

Controlled cortical impact injury. A controlled cortical impact (CCI) device, for example, eCCI Model 6.3; VCU, Richmond, Va. is used to administer a unilateral brain injury as described by Lighthall J., Neurotrauma 5, 1-15 (1988)), the disclosure of which is hereby incorporated by reference in its entirety. Male rats weighing 225-250 g are anesthetized with 4% isoflurane and O₂ and the head of each rat is mounted in a stereotactic frame. The head is held in a horizontal plane. A midline incision is used for exposure, and a 7-8 mm craniectomy is performed on the right cranial vault. The center of the craniectomy is placed at the midpoint between bregma and lambda, ˜3 mm lateral to the midline, overlying the tempoparietal cortex. Animals receive a single impact of 3.1 mm depth of deformation with an impact velocity of 5.8 m/s and a dwell time of 150 ms (moderate-severe injury) at an angle of 10° from the vertical plane using a 6 mm diameter impactor tip, making the impact orthogonal to the surface of the cortex. The impact is made to the parietal association cortex. Sham injuries are performed by anesthetizing the animals, making the midline incision, and separating the skin, connective tissue, and aponeurosis from the cranium. The incision is then closed.

Preparation and intravenous injection of AMDACs. Prior to injection, AMDACs are thawed, washed and suspended in phosphate buffered saline (PBS) vehicle at a concentration of 2×10⁶ cells/mL. Cells are counted and checked for viability via Trypan blue exclusion. Immediately prior to intravenous injection, AMDACs are titrated gently 8-10 times to ensure a homogeneous mixture of cells. AMDACs are injected at both 2 and 24 h after CCI injury at 2 different dosages (CCI+2×10⁶ AMDACs/kg, and CCI+10×10⁶ AMDACs/kg). Therefore, each treatment animal receives 2 separate doses of their assigned AMDACs concentration. CCI injury control animals receive PBS vehicle injection alone at the same designated time points as the cell treated animals.

Rat splenectomy. For all experiments completed with rats after splenectomy, male Sprague Dawley rats are anesthetized as described above and placed in the supine position. A small 3 cm incision is made in the left upper quadrant of the abdomen followed by retraction of the spleen and ligation of the splenic hilum. After removal of the spleen the incision is closed with a running suture. The animals are allowed to recover and acclimate for 72 h after splenectomy. All experiments are then completed 72 h after the original splenectomy.

Evan's blue blood brain barrier (BBB) permeability analysis. Seventy two hours after CCI injury, the rats are anesthetized as described above, and 1 mL (4 cm³/kg) of 3% Evan's blue dye in PBS is injected via direct cannulation of the right internal jugular vein. The animals are allowed to recover for 60 min to allow for perfusion of the dye. After this time, the animals are sacrificed via right atrial puncture and perfused with 4% paraformaldehyde. Next, the animals are decapitated followed by brain extraction. The cerebellum is dissected away from the rest of the cortical tissue. The brain is divided through the midline and the mass of each hemisphere (ipsilateral to injury and contralateral to injury) is measured for normalization. Subsequently, each hemisphere is allowed to incubate overnight in 5 mL of formamide at 50° C. to allow for dye extraction. After centrifugation, 100 μL of the supernatant from each sample is transferred to a 96 well plate (in triplicate) and absorbance is measured at 620 nm. All values are normalized to hemisphere weight.

Cortical immunohistochemistry. BBB integrity is further examined by immunostaining for the tight junction protein occluding, and visualization with fluorescent microscopy (DAPI blue for nuclei and FITC green for occludin). Seventy two hours after CCI injury, 4 groups (uninjured, CCI injury alone, CCI injury+2×10⁶ AMDACs/kg, and CCI injury+10×10⁶ AMDACs/kg) of both rats with intact spleens and rats after splenectomy are sacrificed followed quickly by decapitation. The brains are extracted and both hemispheres (ipsilateral and contralateral to injury) are isolated. The tissue samples are then quickly placed into pre-cooled 2-methylbutane for flash freezing. The samples are transferred to dry ice and stored at −80° C. until the tissue is sectioned. The tissue samples are then placed in Optimal Cutting Temperature compound, for example, Sakura Finetek, Torrance, Calif., and 20 μM cryosections are made through the direct injury area. Direct injury to the vascular architecture is evaluated via staining with an antibody for the tight junction protein occludin (for example, 1:150 dilution, Invitrogen, Carlsbad, Calif.) and appropriate fluorescein isothiocyanate (FITC) conjugated secondary antibody (for example, 1:200 dilution, Invitrogen, Carlsbad, Calif.). After all antibody staining, the tissue sections are counterstained with 4′6-diamidino-2-phenylindole (DAPI) (for example, Invitrogen, Carlsbad, Calif.) for nuclear staining and visualized with fluorescent microscopy.

Splenic immunohistochemistry. In order to track AMDACs in vivo, for example, to determine if administered AMDACs bypass the pulmonary microvasculature and reach the spleen, 4 groups of rats (uninjured, CCI injury alone, CCI injury+2×10⁶ AMDACs/kg, and CCI injury+10×10⁶ AMDACs/kg) undergo either sham injury or CCI injury. Next, the two treatment groups receive injections of quantum dot (for example, QDOT, Qtracker cell labeling kit 525 and 800, Invitrogen, Inc., Carlsbad, Calif.) labeled (per manufacturer's suggested protocol) AMDACs, 2 and 24 h after CCI injury. Six hours after the second QDOT labeled AMDACs infusion, the animals are sacrificed and the spleens removed. The spleens are subsequently placed on a fluorescent scanner (for example, Odyssey Imaging System, Licor Inc., Lincoln, Nebr.) to localize QDOT labeled AMDACs. After the scan is completed, the tissue samples are then quickly placed into pre-cooled 2-methylbutane for flash freezing. The samples are transferred to dry ice and stored at −80° C. until use. Next, the tissue samples are placed in Optimal Cutting Temperature compound (for example, Sakura Finetek, Torrance, Calif.) and 10 μm cryosections are made through the spleens. The tissue sections are stained with 4′6-diamidino-2-phenylindole (DAPI) (Invitrogen, Carlsbad, Calif.) for nuclear staining and both the QDOT labeled AMDACs and splenocytes are visualized with fluorescent microscopy. Furthermore, hematoxylin and eosin staining is performed per manufacturer's suggested protocol to evaluate splenic architecture.

Splenocyte isolation/measurement of splenic mass. Seventy two hours after injury, the animals undergo splenectomy with measurement of splenic mass. The animals are euthanized at this time. Next, the spleens are morselized using a razor blade, washed with basic media (10% FBS and 1% penicillin/streptomycin in RPMI), crushed, and filtered through a 100 μM filter. The effluent sample from the filter is gently titrated 8-10 times and subsequently filtered through a 40 μm filter to remove any remaining connective tissue. The samples are centrifuged at 1000 g for 3 min. Next the supernatant solutions are removed and the samples are suspended in 3 mL of red blood cell lysis buffer (Qiagen Sciences, Valencia, Calif.) and allowed to incubate on ice for 5 min. Subsequently, the samples are washed twice with basic media and centrifuged using the aforementioned settings. The splenocytes are counted and checked for viability via Trypan blue exclusion.

In vivo splenocyte proliferation assay. The percentage of actively proliferating splenocytes (S phase) at the time of sacrifice is measured using, for example, Click-iT™ EdU Flow Cytometry Assay Kit (Invitrogen, Carlsbad, Calif.) according to the manufacturer's suggested protocol. Briefly, splenocytes are harvested at 72 h, and 20 mM of EdU is added to the cells and allowed to incubate for 2 h. Next, the cells are washed and fixed with 4% paraformaldehyde. Cells are permeabilized using Triton-X100 and then the anti-EdU antibody “cocktail” provided by the manufacturer is added. Finally, the cells are washed followed by the addition of Ribonuclease and CellCycle488-Red stain to analyze DNA content.

In vivo splenocyte apoptosis assay. The percentage of apoptotic splenocytes at the time of sacrifice is measured using, for example, an Annexin V stain (BD Biosciences, San Jose, Calif.) according to the manufacturer's suggested protocol. Briefly, after isolation, splenocytes are washed twice with cold PBS. Next, 1×10⁶ cells are incubated with 5 μL of Annexin V and 7-Amino-Actinomycin (7-AAD) for 15 min. Flow cytometry is then used to measure the percentage of apoptotic cells. Quantitative PCR RNA is isolated from splenocytes using, for example, RNEasy columns (Qiagen, Valencia, Calif.) according to manufacturer's specifications. Rat reference RNA (Stratagene, La Jolla, Calif.) is used as a positive control. Synthesis of cDNA is performed with M-MLV reverse transcriptase and random hexamers (Promega, Madison, Wis.). Control reactions are performed without reverse transcriptase to control for genomic DNA contamination. qPCR is performed using, for example, an ABI 7500 with 9600 emulation.

### In Vitro Methods

Splenocyte culture. Splenocytes cultured at a density of 7.5×10⁵ cells/mL are allowed to expand for 72 h in growth media (10% FBS, 1% RPMI with vitamins, 1% sodium pyruvate, 0.09% 2-mercaptoethanol, and 1% penicillin/streptomycin in RPMI) stimulated with 2 μg concanavalin A.

Splenocyte characterization. The isolated splenocytes are analyzed with flow cytometry to determine the monocyte, neutrophil, and T cell populations. Monocytes and neutrophils are measured using antibodies to CD200 and CD11b/CD18, respectively. The splenocyte T cell populations are labeled using CD3, CD4, and CD8 antibodies. All staining is completed in accordance with manufacturer's suggested protocol.

Proliferation assay in vitro. The percentage of CD4+ splenocytes actively proliferating (S phase) after culture in stimulated growth media is measured using, for example, Click-iT™ EdU Flow Cytometry Assay Kit (Invitrogen, Carlsbad, Calif.) following the manufacturer's suggested protocol. Briefly, splenocytes are cultured for 72 h as previously described in growth media stimulated with 2 μg concanavalin A at a density of 7.5×10⁵ cells/mL. 20 mM of EdU is added and allowed to incubate for 1 h. Next, the cells are washed with 4% bovine serum in DMEM (4% FBS) and CD4-PE is added to gate the T cell population of interest. After 30 min of incubation, the cells are washed and fixed with 4% paraformaldehyde. Cells are permeabilized using Triton-X100 and then the anti-EdU antibody “cocktail” provided by the manufacturer is added. Finally, the cells are washed followed by the addition of Ribonuclease and CellCycle488-Red stain to analyze DNA content.

Splenocyte cytokine production in vitro. After culture in stimulated growth media, production of the anti-inflammatory cytokines IL-4 and IL-10 was quantified by flow cytometry using, for example, a BD Cytometric Bead Array flex set (BD Biosciences, San Jose, Calif.) following manufacturer's suggested protocol.

### 6.8 Example 8

### Use of AMDACS for Tissue Remodeling

This example demonstrates how AMDACs can be used to modulate fibrosis and remodel tissue.

Using ELISA and multiplex assays, AMDAC-conditioned medium was compared with medium conditioned normal human dermal fibroblasts (NHDF) to assess the secretion profiles of the two cell types. AMDACs were determined to secrete more follistatin than the amount of follistatin secreted by the NHDF. AMDACs also were determined to secrete more hepatocyte growth factor (HGF) than the amount of HGF secreted by the NHDF. Additionally, AMDACs were determined to secrete matrix metalloproteinase (MMP) 1, MMP2, MMP7, and MMP10.

The determination that AMDACs secrete high levels of both follistatin and HGF relative to NHDF, and that AMDACs also secrete MMP1, MMP2, MMP7, and MMP10, indicates that AMDACs can modulate fibrosis in vivo and thus can be useful in methods involving tissue remodeling, e.g., methods as described herein.

### 6.9 Example 9

### Methods of Treatment Using Amnion Derived Adherent Cells

6.9.1 Treatment of SCI Using AMDACs

An individual presents with spinal cord injury (SCI) and is experiencing loss of sensory and/or motor function. The individual is administered 2.5×108 to 1×1010 cells of a population of OCT-4−, CD49f+ amnion derived adherent cells (AMDACs) in a 0.9% NaCl solution intravenously. The individual is monitored over the subsequent month to assess reduction in one or more of the symptoms. The individual is additionally monitored over the course of the following year, and AMDACs in the same dose are administered as needed, e.g., if symptoms return or increase in severity.

6.9.2 Treatment of SCI Using AMDACs

An individual presents with spinal cord injury (SCI) and is experiencing loss of sensory and/or motor function. The individual is administered 1×106 to 1×107 cells of a population of OCT-4−, CD49f+ amnion derived adherent cells (AMDACs) in a 0.9% NaCl at the site of spinal cord injury. The individual is monitored over the subsequent month to assess reduction in one or more of the symptoms. The individual is additionally monitored over the course of the following year, and AMDACs in the same dose are administered as needed, e.g., if symptoms return or increase in severity.

6.9.3 Treatment of TBI Using AMDACs

An individual presents with traumatic brain injury (TBI) and is experiencing memory loss, poor attention/concentration, and/or dizziness/loss of balance. The individual is administered 2.5×108 to 1×1010 cells of a population of OCT-4−, CD49f+ amnion derived adherent cells (AMDACs) in a 0.9% NaCl solution intravenously. The individual is monitored over the subsequent month to assess reduction in one or more of the symptoms. The individual is additionally monitored over the course of the following year, and AMDACs in the same dose are administered as needed, e.g., if symptoms return or increase in severity.

6.9.4 Treatment of TBI Using AMDACs

An individual presents with traumatic brain injury (TBI) and is experiencing memory loss, poor attention/concentration, and/or dizziness/loss of balance. The individual is administered 1×106 to 1×107 cells of a population of OCT-4−, CD49f+ amnion derived adherent cells (AMDACs) in a 0.9% NaCl intracranially. The individual is monitored over the subsequent month to assess reduction in one or more of the symptoms. The individual is additionally monitored over the course of the following year, and AMDACs in the same dose are administered as needed, e.g., if symptoms return or increase in severity.

### EQUIVALENTS

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method of treating an individual having or at risk of developing a disease, disorder or condition of the central nervous system, comprising administering to the individual a therapeutically effective amount of amnion derived adherent cells (AMDACs), or culture medium conditioned by amnion derived adherent cells, wherein the therapeutically effective amount is an amount sufficient to cause a detectable improvement in one or more symptoms of said disease, disorder or condition, and wherein said AMDACs are OCT-4⁻ as determinable by RT-PCR, and are adherent to tissue culture plastic.

2. The method of claim 1, wherein said AMDACs are (i) OCT-4− as determinable by RT-PCR, and CD49f+, CD105+, and CD200+ as determinable by flow cytometry, (ii) positive for VEGFR1/Flt-1 (vascular endothelial growth factor receptor 1) and VEGFR2/KDR (vascular endothelial growth factor receptor 2), as determinable by immunolocalization, (iii) CD90+ and CD117− as determinable by flow cytometry, and HLA-G−, as determinable by RT-PCR, or (iv) OCT-4− and HLA-G−, as determined by RT-PCR, and CD49f+, CD90+, CD105+, and CD117− as determinable by flow cytometry.

3. (canceled)

4. (canceled)

5. (canceled)

6. The method of claim 1, wherein said AMDACs are additionally one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, or CXCR4⁻ (chemokine (C—X—C motif) receptor 4) as determinable by immunolocalization; or are additionally CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization.

7. (canceled)

8. The method of claim 1, wherein said AMDACs are OCT-4⁻, as determinable by RT-PCR, and CD49f⁺, HLA-G⁻, CD90⁺, CD105⁺, CD117⁻, and CD200⁺, as determinable by immunolocalization, and wherein said AMDACs:
(a) express one or more of CD9, CD10, CD44, CD54, CD98, CD200, Tie-2, TEM-7, VEGFR1/Flt-1, or VEGFR2/KDR (CD309), as determinable by immunolocalization;
(b) lack expression of CD31, CD34, CD38, CD45, CD133, CD143, CD144, CD146, CD271, CXCR4, HLA-G, or VE-cadherin, as determinable by immunolocalization;
(c) lack expression of SOX2, as determinable by RT-PCR;
(d) express mRNA for ACTA2, ADAMTS1, AMOT, ANG, ANGPT1, ANGPT2, ANGPTL1, ANGPTL2, ANGPTL4, BAI1, c-myc, CD44, CD140a, CD140b, CD200, CD202b, CD304, CD309, CEACAM1, CHGA, COL15A1, COL18A1, COL4A1, COL4A2, COL4A3, Connexin-3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, Galectin-1, GRN, HGF, HEY1, HSPG2, IFNB1, IL8, IL12A, ITGA4, ITGAV, ITGB3, KLF-4, MDK, MMP2, MYOZ2, NRP2, PDGFB, PF4, PGK1, PROX1, PTN, SEMA3F, SERPINB5, SERPINC1, SERPINF1, TGFA, TGFB1, THBS1, THBS2, TIE1, TIMP2, TIMP3, TNF, TNNC1, TNNT2, TNFSF15, VASH1, VEGF, VEGFB, VEGFC, or VEGFR1/FLT1;
(e) produce one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A;
(f) secrete vascular endothelial growth factor (VEGF), hepatocyte growth factor (HGF), interleukin-8 (IL-8), monocyte chemotactic protein-3 (MCP-3), FGF2, Follistatin, G-CSF, EGF, ENA-78, GRO, IL-6, MCP-1, PDGF-BB, TIMP-2, uPAR, or galectin-1 into culture medium in which the AMDACs grows;
(g) express micro RNAs miR-17-3p, miR-18a, miR-18b, miR-19b, miR-92, or miR-296 at a higher level than an equivalent number of bone marrow-derived mesenchymal stem cells;
(h) express micro RNAs miR-20a, miR-20b, miR-221, miR-222, miR-15b, or miR-16 at a lower level than an equivalent number of bone marrow-derived mesenchymal stem cells;
(i) express miRNAs miR-17-3p, miR-18a, miR-18b, miR-19b, miR-92, miR-20a, miR-20b, miR-296, miR-221, miR-222, miR-15b, or miR-16; or
(j) express increased levels of CD202b, IL-8 or VEGF when cultured in less than about 5% O₂ compared to expression of CD202b, IL-8 or VEGF when cultured under 21% O₂.

9. The method of claim 8, wherein said AMDACs are OCT-4⁻, as determinable by RT-PCR, and CD49f⁺, HLA-G⁻, CD90⁺, CD105⁺, and CD117⁻, as determinable by immunolocalization, and wherein said AMDACs:
(a) express CD9, CD10, CD44, CD54, CD98, CD200, Tie-2, TEM-7, VEGFR1/Flt-1, and VEGFR2/KDR (CD309), as determinable by immunolocalization;
(b) lack expression of CD31, CD34, CD38, CD45, CD133, CD143, CD144, CD146, CD271, CXCR4, HLA-G, and VE-cadherin, as determinable by immunolocalization;
(c) lack expression of SOX2, as determinable by RT-PCR;
(d) express mRNA for ACTA2, ADAMTS1, AMOT, ANG, ANGPT1, ANGPT2, ANGPTL1, ANGPTL2, ANGPTL4, BAI1, c-myc, CD44, CD140a, CD140b, CD200, CD202b, CD304, CD309, CEACAM1, CHGA, COL15A1, COL18A1, COL4A1, COL4A2, COL4A3, Connexin-3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, Galectin-1, GRN, HGF, HEY1, HSPG2, IFNB1, IL8, IL12A, ITGA4, ITGAV, ITGB3, KLF-4, MDK, MMP2, MYOZ2, NRP2, PDGFB, PF4, PGK1, PROX1, PTN, SEMA3F, SERPINB5, SERPINC1, SERPINF1, TGFA, TGFB1, THBS1, THBS2, TIE1, TIMP2, TIMP3, TNF, TNNC1, TNNT2, TNFSF15, VASH1, VEGF, VEGFB, VEGFC, and VEGFR1/FLT1 as determinable by RT-PCR;
(e) produce the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A;
(f) secrete VEGF, HGF, IL-8, MCP-3, FGF2, Follistatin, G-CSF, EGF, ENA-78, GRO, IL-6, MCP-1, PDGF-BB, TIMP-2, uPAR, and Galectin-1 into culture medium in which the cell grows;
(g) express micro RNAs miR-17-3p, miR-18a, miR-18b, miR-19b, miR-92, and miR-296 at a higher level than an equivalent number of bone marrow-derived mesenchymal stem cells;
(h) express micro RNAs miR-20a, miR-20b, miR-221, miR-222, miR-15b, and miR-16 at a lower level than an equivalent number of bone marrow-derived mesenchymal stem cells;
(i) express miRNAs miR-17-3p, miR-18a, miR-18b, miR-19b, miR-92, miR-20a, miR-20b, miR-296, miR-221, miR-222, miR-15b, and miR-16; or
(j) express increased levels of CD202b, IL-8 and/or VEGF when cultured in less than about 5% O₂, compared to expression of CD202b, IL-8 and/or VEGF under 21% O₂.

10. The method of claim 1, comprising additionally administering a second type of stem cells to said individual.

11. The method of claim 10, wherein said second type of stem cells are embryonic stem cells, stem cells isolated from peripheral blood, stem cells isolated from placental blood, stem cells isolated from placental perfusate, non-AMDAC stem cells isolated from placental tissue, stem cells isolated from umbilical cord blood, umbilical cord stem cells, bone marrow-derived mesenchymal stem cells, adipose-derived stem cells, hematopoietic stem cells, or somatic stem cells.

12. The method of claim 1, wherein said disease, disorder or condition is a spinal cord injury.

13. The method of claim 12, wherein the spinal cord injury is caused by (i) destruction from direct trauma or compression by bone fragments or disc material.

14. (canceled)

15. The method of claim 1, wherein said disease, disorder or condition is (i) spinal shock resulting from a spinal cord injury, (ii) neurogenic shock resulting from a spinal cord injury, (iii) autonomic dysreflexia resulting from a spinal cord injury, or (iv) edema resulting from a spinal cord injury.

16. (canceled)

17. (canceled)

18. (canceled)

19. The method of claim 1, wherein said disease, disorder or condition is selected from the group consisting of central cord syndrome, Brown-Séquard syndrome, anterior cord syndrome, conus medullaris syndrome, and cauda equina syndrome.

20. The method of claim 12, wherein the spinal cord injury is at one or more of the cervical vertebrae, thoracic vertebrae, lumbar vertebrae, or sacral vertebrae; or wherein the spinal cord injury is to one or more of the cervical cord, thoracic cord, lumbrosacral vertebrae, conus, occiput, or one or more nerves of the cauda equina.

21. (canceled)

22. The method of claim 12, wherein said one or more symptoms comprises (i) loss or impairment of motor function, sensory function, or motor and sensory function, in the cervical, thoracic, lumbar or sacral segments of the spinal cord; (ii) loss or impairment of motor function, sensory function, or motor and sensory function, in the arms, trunk, legs or pelvic organs; or (iii) numbness in one or more of dermatomes C1, C2, C3, C4, C5, C6, C7, T1, T2, T3, T4, T5, T6, T7, T8, T9, T10, T11, T12, L1, L2, L3, L4 or L5.

23. (canceled)

24. (canceled)

25. The method of claim 12, wherein the therapeutically effective amount of amnion derived adherent cells, or culture medium conditioned by amnion derived adherent cells is administered to the individual within 14 days of the spinal cord injury.

26. The method of claim 12, comprising administering a second therapeutic agent to said individual, wherein said second therapeutic agent is a corticosteroid, a neuroprotective agent, an immunomodulatory or immunosuppressant agent, or an anticoagulant.

27. (canceled)

28. The method of claim 1, wherein said disease, disorder or condition is a traumatic brain injury.

29. The method of claim 28, wherein the traumatic brain injury is (i) an injury to the frontal lobe, parietal lobe, occipital lobe, temporal lobe, brain stem, or cerebellum; (ii) a mild traumatic brain injury; or (iii) a moderate to severe traumatic brain injury.

30. (canceled)

31. (canceled)

32. The method of claim 28, wherein said symptom is one or more of: headache, difficulty thinking, memory problems, attention deficits, mood swings and frustration, fatigue, visual disturbances, memory loss, poor attention/concentration, sleep disturbances, dizziness/loss of balance, irritability, emotional disturbances, feelings of depression, seizures, nausea, loss of smell, sensitivity to light and sounds, mood changes, getting lost or confused, slowness in thinking, difficulties with attention, difficulties with concentration, distractibility, difficulties with memory, slowness of speed of processing, confusion, perseveration, impulsiveness, difficulties with language processing, difficulties with speech and language, not understanding the spoken word (receptive aphasia), difficulty speaking and being understood (expressive aphasia), slurred speech, speaking very fast or very slow, problems reading, and problems writing, difficulties with interpretation of touch, temperature, movement, limb position and fine discrimination, difficulty with the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), a decrease or loss of hearing, ringing in the ears (tinnitus), increased sensitivity to sounds, loss or diminished sense of smell (anosmia), loss or diminished sense of taste, seizures, convulsions associated with epilepsy, physical paralysis/spasticity, chronic pain, loss of control of bowel and bladder sleep disorders, loss of stamina, appetite changes, dysregulation of body temperature, menstrual difficulties, social-emotional difficulties, dependent behaviors, lack of emotional ability, lack of motivation, irritability, aggression, depression, disinhibition, and lack of awareness.

33. (canceled)

34. The method of claim 28, comprising administering a second therapeutic agent to said individual, wherein said second therapeutic agent is an anti-seizure drug, an antidepressant, amantadine, methylphenidate, bromocriptine, carbamamazapine or amitriptyline.

35. (canceled)

36. The method of claim 1, wherein the therapeutically effective amount of amnion derived adherent cells, or culture medium conditioned by amnion derived adherent cells is administered to the individual by a route selected from the group consisting of intravenous, intraarterial, intraperitoneal, intraventricular, intrasternal, intracranial, intramuscular, intrasynovial, intraocular, intravitreal, intracerebral, intracerebroventricular, intrathecal, intraosseous infusion, intravesical, transdermal, intracisternal, epidural, or subcutaneous administration; or wherein the therapeutically effective amount of amnion derived adherent cells, or culture medium conditioned by amnion derived adherent cells is administered to the individual directly into the site of the injury.

37. (canceled)
